(19) United States
(12) Patent Application Publication
(10) Pub. No.: US 2025/0327079 A1
(43) Pub. Date: Oct. 23, 2025

(54) METHOD FOR CARDIAC REPAIR

(71) Applicant: The Regents of the University of California, Oakland, CA (US)

(72) Inventors: Sylvia Evans, Del Mar, CA (US); Paola Cattaneo, Frankfurt (DE); Nuno Guimarães-Camboa, Frankfurt (DE)

(21) Appl. No.: 18/857,765

(22) PCT Filed: Apr. 19, 2023
(86) PCT No.: PCT/US2023/019079

§ 371 Date: Oct. 17, 2024

Related U.S. Application Data
(60) Provisional application No. 63/332,984, filed on Apr. 20, 2022.

(51) Int. Cl. C12N 15/113

(52) U.S. Cl. CPC C12N 15/1137; C12Y 201/01; C12N 2310/14; C12N 2310/531

(57) ABSTRACT

This disclosure provides methods to modulate cardiac regeneration in a mammalian cardiac cell or progenitor, comprising contacting the mammalian cardiac cell with a DOT1L gene modulator.

### CROSS-REFERENCE TO RELATED APPLICATION

This application claims priority under 35 U.S.C. § 119(e) to U.S. Provisional Application No. 63/332,984, filed Apr. 20, 2022, the contents of which are incorporated herein by reference in their entireties.

### STATEMENT OF GOVERNMENT SUPPORT

This invention was made with government support under HL123747 and HL144984, awarded by the National Institutes of Health (NIH). The government has certain rights in the invention.

### BACKGROUND

Chemotherapeutic reagents have adverse effect on the heart. Pathways that lead to uncontrolled growth of cancer cells are also important for heart function and countering oncological drugs having detrimental effect on the heart. Mammalian cardiomyocytes withdraw from the cell cycle in the neonatal period. Because terminally differentiated adult cardiomyocytes cannot divide, the heart cannot regenerate. In this mutation driven method, cells do not experience the same cell cycle withdrawal. Inhibiting dot1l function can have result of having cardiomyocytes continue to proliferate. Thus, a need exists in the art to discover strategies to promote cardiomyocyte proliferation to enable cardiac regeneration. This disclosure satisfies this need and also provides information regarding the detrimental effects on general health caused by oncologic and similar drugs.

### SUMMARY OF THE DISCLOSURE

Cardiomyopathy and heart failure arise from loss of postnatal cardiomyocytes. This disclosure provides methods and compositions to replace lost cardiomyocytes by stimulating cell cycle re-entry of the remaining cardiomyocytes. Epigenetic modification of proteins that bind to DNA (histone and others) control gene expression programs that lead to development of tumors. Therefore, small molecule inhibitors of specific epigenetic enzymes have been previously developed for targeting leukemia.

Mechanisms by which specific histone modifications regulate distinct gene regulatory networks remain little understood. Applicant investigated how H3K79me2, a modification catalyzed by DOT1L and previously considered a general transcriptional activation mark, regulates gene expression in mammalian cardiogenesis. Early embryonic cardiomyocyte ablation of Dot1l revealed that H3K79me2 does not act as a general transcriptional activator, but rather regulates highly specific gene regulatory networks at two critical cardiogenic junctures: left ventricle patterning and postnatal cardiomyocyte cell cycle withdrawal. Mechanistic analyses revealed that H3K79me2 in two distinct domains, gene bodies and regulatory elements, synergized to promote expression of genes activated by DOT1L. Surprisingly, these analyses also revealed that H3K79me2 in specific regulatory elements contributed to silencing genes usually not expressed in cardiomyocytes. As DOT1L mutants had increased numbers of postnatal mononuclear cardiomyocytes and prolonged cardiomyocyte cell cycle activity, controlled inhibition of DOT1L might be a strategy to promote cardiac regeneration post-injury.

Accordingly, Applicant provides herein a method to modulate cardiogenesis in a mammalian cardiac cell or mammalian cardiac progenitor cell, comprising, or consisting essentially of, or yet further consisting of contacting the cell with a DOT1L gene modulator. The DOT1L gene modulator can be an agent that upregulates DOT1L gene expression or an agent that downregulates or abolishes DOT1L gene expression or function. The contacting can be in vitro or in vivo. The gene modulator that upregulates can be a polynucleotide encoding a DOT1L protein. A gene modulator can also be a system that reduces or abrogates endogenous DOT1L gene expression or function. Non-limiting examples of such include a chemical inhibitor of DOT1L activity, shRNA that targets DOT1L under the control of a cardiac-specific promoter, siRNA that targets DOT1L, or CRISPR gene editing that downregulates DOT1L. The mammalian cardiac cell can be a cell selected from a canine cardiac cell, an equine cardiac cell, a feline cardiac cell, a murine cardiac cell or a human cardiac cell.

Also provided is a method of promoting cardiac regeneration or de novo cell cycle of a post-mitotic mammalian cardiac cell, comprising, or consisting essentially of, or yet further consisting of contacting the cardiac cell with an agent that inhibits expression of an endogenous DOT1L gene or function in the cell, thereby promoting cardiac regeneration in the cell or de novo cell cycle of a post-mitotic cardiac cell. The contacting can be in vitro or in vivo. In one aspect, the cardiac cell is a cell post-injury. In one embodiment, the agent reduces or abrogates endogenous DOT1L gene expression in the cell. Non-limiting examples of such include a chemical inhibitor of DOT1L activity, shRNA that targets DOT1L under the control of a cardiac-specific promoter, siRNA that targets DOT1L, or CRISPR gene editing that downregulates DOT1L.

Also provided is a method for promoting cardiac regeneration in a subject or for treating cardiac disease or injury in a subject in need thereof, comprising, or consisting essentially of, or yet further consisting of administering to the subject an agent that inhibits expression of an endogenous DOT1L gene in a subject's cardiac cell in the subject, thereby promoting cardiac regeneration or treating cardiac disease or injury in the subject. The subject can be a mammal or such as a human patient. In one embodiment, the cardiac cell is a cell post-injury. In a further aspect, the agent reduces or abrogates endogenous DOT1L gene expression in the cell, non-limiting examples of such include a chemical inhibitor of DOT1L activity, shRNA that targets DOT1L under the control of a cardiac-specific promoter, siRNA that targets DOT1L, or CRISPR gene editing that downregulates DOT1L.

Administration of the agent can be administered locally or systemically. In one aspect, the agent is administered locally via intracardiac injection or reperfusion.

Kits comprising the agents and instructions for use in the methods as described herein are further provided.

### BRIEF DESCRIPTION OF THE DRAWING

FIGS. 1A-1J: Cardiomyocyte-specific ablation of DOT1L from early developmental timepoints results in enlarged hearts and peri-natal lethality. (FIG. 1A) qPCR analysis using a primer within the floxed exon of Dot1l mRNA showing efficient ablation of this gene in E12.5 cKO FACS-sorted CMs (N=3 biological replicates, unpaired t-test, two-sided P=0.0271). Western blot (FIG. 1B) and respective quantification (FIG. 1C) showing strongly reduced H3K79me2 levels in E14.5 hearts upon ablation of Dot1L (N=3 biological replicates, unpaired t-test, two-sided P=0.0004). (FIG. 1D) Kaplan-Meier survival curves showing postnatal lethality of Dot1L cKOs. (FIG. 1E) Whole mount images of postnatal day (P) 1, P5 and P10 hearts in Ctrls and Dot1L cKOs representative of the enlarged heart phenotype of Dot1L cKOs (scale bars=1 mm). (FIG. 1F) Graph representing a significant increase in heart weight/body weight ratio (HW/BW (mg/g)) in Dot1L cKO vs Ctrl in all stages analyzed. (P1 Ctrl N=36, P1 cKO N=30, unpaired t-test, two-sided P<0.0001; P5 Ctrl N=5, P5 cKO N=9, unpaired t-test, two-sided P=0.0223; P10 Ctrl N=16, P10 cKO N=9 biological replicates, unpaired t-test, two-sided P=0.0002). (FIG. 1G) Confocal images showing no alterations in sarcomere organization and myofiber orientation in Dot1L cKOs. αSarcomeric Actinin in light gray, Myomesin in dark gray, DAPI (4′, 6-diamidin-2-phenylindol) in medium gray (scale bar=10 μm for lower magnification images on the left and 1 μm for higher magnification images on the right) (FIG. 1H) Immunofluorescence time course depicting the dynamics of phenotypic manifestations in Dot1L cKOs in embryonic (E) and postnatal (P) stages. DAPI in medium gray, Vimentin in light gray and lineage traced xMlc2-Cre; tdTomato CMs in dark gray (scale bar=1 mm). i-j) Assessment of CM length (left), width (middle) and ratio of CM length/width (right) at P5 (FIG. 1I) and P10 (FIG. 1J) indicated no major changes in CM size. Measurements were performed on isolated CMs (mean of 413 CMs counted per heart from N=3 biological replicates, unpaired t-test, two-sided P=0.0096 for P10 CM width). In all graphs Ctrl indicates control mice (XMlc2-Cre; Dot1L fl/+), cKO indicates mutant mice (XMlc2-Cre; Dot1L Δ/fl). Data is presented as mean±SEM; * represents P<0.05, ** P<0.01.

FIGS. 2A-2E: Echocardiographic and electrocardiographic defects in Dot1L cKO hearts. (FIGS. 2A-2B) Echocardiographic analyses conducted at P5 (FIG. 2A) and P10 (FIG. 2B) revealed significant defects in Dot1L cKO hearts at both timepoints, including increased left ventricular inner diameter, both in diastole (LVIDd) and systole (LVIDs), reduced fractional shortening (FS), and increased diastolic left ventricle mass to body weight ratios (LVMd/BW). Dot1L cKOs also exhibited increased diastolic left ventricular posterior wall thickness (LVPWd) at P5 and increased diastolic intra-ventricular septum thickness (IVSd) at P10. Additionally, Dot1L cKOs displayed reduced heart rate (HR) at both timepoints. (P5 Ctrl N=13, P5 cKO N=13, P10 Ctrl N=12, P10 cKO N=11 biological replicates; unpaired t-test, two-sided P5 LVIDd P<0.0001, P5 LVIDs P<0.0001, P5 FS P<0.0001, P5 LVPWd P=0.0029, P5 LVMd/BW P=0.0017, P5 HR P<0.0001; P10 LVIDd P<0.0001, P10 LVIDs P<0.0001, P10 FS P<0.0001, P10 IVSd P=0.0083, P10 LVMd/BW P=0.0002, P10 HR P<0.0005). (FIG. 2C) Representative ECG tracks from Ctrl and Dot1L cKO (FIGS. 2D-2E) ECG measurements revealed multiple defects in Dot1L cKOs both at P5 (FIG. 2D) and P10 (FIG. 2E), including increased and irregular R-R and QRS intervals. At P5 Dot1L cKOs also displayed significantly increased P-R intervals, however, this difference could not be detected in P10 mutants. (P5 N=7, P10 N=8 biological replicates, unpaired t-test, two-sided P5 R-R P=0.004, P5 QRS P=0.0164, P5P-R P=0.0017, P10 R-R P=0.0024, P10 QRS P=0.001, P10P-R P=0.2279). The left part of the graph represents the mean±SD of multiple beats measured for each mouse. The right graph represents the mean±SD of multiple biological replicates. Data is presented as mean±SD; * represents P≤0.05, **P≤0.01. Source data are provided as a Source Data file.

FIGS. 3A-3I: DOT1L is required in cardiomyocytes for chamber-specific gene expression. (FIG. 3A) Pie chart representing the number of genes down-(log₂FC≤−0.5; FDR≤0.05) and up-regulated (log₂FC≥0.5; FDR≤0.05) in Dot1L cKO CMs at E16.5. (FIG. 3B) Quartile distribution of gene expression in CMs at E16.5. Genes downregulated (Down) in Dot1L cKO were expressed at a high level in control CMs, whereas the majority of upregulated genes (Up) belonged to the bottom quartile of expression. Genes not significantly modulated (Unch) were evenly distributed across quartiles of expression. Data are shown as stacked percentage bar graph. (FIG. 3C) Heatmap showing the expression of the top 25 transcription regulators downregulated in Dot1L cKO CMs at E16.5, highlighting that multiple chamber-specific transcription regulators were significantly downregulated. (FIGS. 3D and 3E) RNA-scope analyses (FIG. 3D) and respective quantification (FIG. 3E) validating blunted expression of Hand1 in Dot1L cKOs both at E10.5 and E16.5. (N=3 biological replicates; unpaired t-test, two-sided, E10.5 RV P=0.0042, E10.5 LV P=0.0020, E16.5 LV P=0.0003) (FIG. 3F) Quantification of RNA-scope analysis validating no changes in expression of Hand2 in Dot1L cKOs both at E10.5 and E16.5 (RNA-scope images presented in FIG. 11) (N=3 biological replicates). (FIGS. 3G-3H) RNA-scope analyses (FIG. 3G) and respective quantification (FIG. 3H) validating reduced levels of Irx4 in Dot1L cKOs both at E10.5 and E16.5. (N=3 biological replicates; unpaired t-test, two-sided, E10.5 LV P=0.0153, E16.5 RV P=0.0006, E16.5 LV P=0.0005) (FIG. 31) Quantification of RNA-scope analysis validating reduced levels of Smyd1 in Dot1L cKOs both at E10.5 and E16.5 (RNA-scope images presented in FIG. 10). (N=3 biological replicates; unpaired t-test, two-sided, E16.5 RV P=0.0057, E16.5 LV P=0.008) For panels (FIG. 3D) and (FIG. 3G) scale bars=250 μm for low magnification panels and 50 μm for high magnification images. In panels (FIG. 3E), (FIG. 3F), (FIG. 3H) and (FIG. 31) data is presented as mean±SEM; * represents P0.05, **P<0.01. (N=3 biological replicates).

FIGS. 4A-4K: DOT1L controls transcription of target genes via a combination of H3K79me2 in gene bodies and regulatory elements. (FIG. 4A) Volcano plot displaying H3K79me2 ChIP-seq peaks significantly enriched in E16.5 Dot1L cKO vs Ctrl CMs. (FIG. 4B) Pie chart indicating the genomic distribution of differential H3K79me2 ChIP-seq peaks in E16.5 CMs. (FIG. 4C) Metagene profiles showing the average distribution of H3K79me2 input-normalized density relative to Transcription Start Site (TSS) and Transcription Termination Site (TTS) with ±2 Kb flanking regions. (FIG. 4D) Fraction of gene body covered with H3K79me2 in downregulated genes (left graph) and in upregulated genes (right graph). (FIG. 4E) Graph representing the percentage of down- and up-regulated genes in E16.5 cKO CMs with (Coverage≥50 reads and Fraction of gene body≥0.2) or without (Coverage<50 reads or Fraction of gene body<0.2) gene body H3K79me2 in E16.5 Ctrl CMs. (FIG. 4F) Percentage of genes with H3K79me2 in the gene body (GB) or without H3K79me2 in the gene body across the distinct quartiles of expression. This modification was abundant amongst highly expressed genes (4^{th} quartile of RNA expression) and progressively decreased towards the lower quartiles of expression. Globally more than half (58%) of all genes expressed in E16.5 CMs had gene body H3K79me2. (FIG. 4G) Heatmap indicating the number of total and shared regions between H3K27ac ChIP-seq peaks in Ctrl CMs, differential H3K79me2 ChIP-seq peaks and promoters (±200 bp around 5′TSS) in E16.5 CMs. Different intensities of colors indicate the fraction (%) of shared peaks. (FIG. 411) UpSet plot indicating the number and percentage of genes up and downregulated in cKOs versus Ctrls with or without H3K79me2 in gene body (GB) and/or regulatory elements (REs) in E16.5 CMs. i-j) Metascape pathway analysis of genes downregulated with H3K79me2 in GB and K79-REs (FIG. 4I) and upregulated without gene body H3K79me2 but with K79-REs (FIG. 4J) in Dot1L cKO versus Ctrl E16.5 CMs. Top 5 enriched categories are shown, sorted by Log₁₀ P value. (FIG. 4K) Motif enrichment analysis ranking transcription factors (TFs) enriched in K79-REs associated with upregulated genes without H3K79me2 in GB versus K79-REs associated with downregulated genes with H3K79me2 in GB.

FIGS. 5A-5G: H3K27ac Relationship to H3K79me2. (FIG. 5A) Volcano plot displaying H3K27ac ChIP-seq peaks significantly enriched in E16.5 Dot1L cKO vs Ctrl CMs. (FIG. 5B) Heatmap indicating the number of total and shared regions between differential H3K27ac ChIP-seq peaks, differential H3K79me2 ChIP-seq peaks and promoters (±200 bp around 5′TSS) in E16.5 CMs. Different intensities of colors indicate the fraction (%) of shared peaks. (FIG. 5C) Graph representing the percentage of differential H3K27ac ChIP-seq peaks in E16.5 cKO vs Ctrl CMs overlapping (orange) or not overlapping (grey) with H3K79me2 ChIP-seq peaks. H3K27ac downregulated peaks are more often differential for H3K79me2 than H3K27ac upregulated peaks (oddsratio=0.004; p-value=5.93e-212). (FIGS. 5D-5E) UpSet plots indicating the number and percentage of genes down-(FIG. 5D) and upregulated (FIG. 5E) in E16.5 Dot1L cKO versus Ctrl CMs with or without H3K79me2 in gene body (GB) and/or regulatory elements (REs) and with or without differential H3K27ac REs. (FIG. 5F) Browser tracks displaying H3K79me2 and H3K27ac ChIP-seq profiles of Ctrl (gray scaled) and Dot1L cKO (gray scaled) E16.5 CMs in the genomic region containing the Hand1 locus. Loops display all regulatory interactions between REs and the Hand1 gene, as identified by the ABC analysis. Gray loops identify interactions with REs without H3K79me2, loops identify interactions with K79-REs, whereas other loops represent interactions with K79-REs that additionally have H3K27ac REs differentially enriched between Dot1L Ctrls and cKOs. For reference, all REs are displayed in the middle lane in black or grey, regardless of their regulatory association with the Hand1 gene. Differential H3K27ac REs are indicated in the bottom lane in gray scaled when they overlap a K79-RE or darker gray when they do not. (FIG. 5G) Diagrammatic representation summarizing the involvement of DOT1L in mammalian cardiogenesis. Genes directly regulated by DOT1L (via H3K79me2) are highlighted in red.

FIGS. 6A-61: Neonatal Dot1L cKO cardiomyocytes fail to undergo cell cycle withdrawal. (FIGS. 6A-6B) Representative FACS analysis (FIG. 6A) and respective quantification (FIG. 6B) showing significantly increased EdU incorporation within P1 CMs (tdTomato+ cells) of Dot1L cKO vs Ctrl hearts (Ctrl N=6, cKO N=3 biological replicates, unpaired t-test, two-sided, P=0.0025). (FIGS. 6C-6D) Representative immunofluorescence images (FIG. 6C), and respective quantification (FIG. 6D) showing significantly increased rates of EdU incorporation in P10 CMs isolated from Dot1L cKO vs Ctrl hearts. DAPI in blue, endogenous tdTomato signal driven by xMlc2-Cre; Rosa26-tdTomato in red and EdU in white. (Scale bar=100 μm; Mean of 755 CMs counted per heart from N=3 biological replicates, unpaired t-test, two-sided, P<0.0001). (FIG. 6E) Quantification of relative percentage of mononuleated (Mono), binuclated (Bi) or multinucleated (>2) CMs in P10 Dot1L Ctrls and cKOs. At P10, Dot1L cKO hearts had more mononucleated (Mono) and less binucleated (Bi) CMs than littermate Ctrls (mean of 697 CMs counted per heart from N=6 Ctrl and N=7 cKO biological replicates, unpaired t-test, two-sided, Mono P=0.0002, Bi P<0.0001, >2P=0.0007). (FIG. 6F) Quantification of percentage of EdU+ CMs within mononucleated (left graph) and binucleated (right graph) CMs of P10 Dot1L Ctrls and cKOs (Mean of 755 CMs counted per heart from N=3 biological replicates, unpaired t-test, two-sided P=0.0006). (FIG. 6G, FIG. 6H, FIG. 61) Immunofluorescence images (FIG. 6G) and respective quantification (FIG. 6H, FIG. 61) of P10 Dot1L Ctrl and cKO hearts on a Rosa26-Fucci2A background. Lighter gray-only nuclei represent CMs in G1; medium gray scale nuclei represent CMs in G1/S; lighter gray-only nuclei correspond to CMs in S/G2/M, pH3 staining in white. Dot1L cKO hearts had a significantly higher percentage of CMs in G1/S and in S/G2/M compared to Ctrls, (h, mean of 2070 CMs counted per heart from N=3 biological replicates, unpaired t-test, two-sided G1 P=0.0256, G1/S P=0.0308, S/G2/M P=0.0126) and of phophoHistone3+(pH3) CMs (FIG. 61), mean of 1019 CMs counted per heart from N=3 biological replicates, unpaired t-test, two-sided P=0.0014). (Scale bar=50 μm; sections have been quantified from all the compartments of the heart). In all graphs from (FIG. 6B) to (FIG. 61) data is presented as mean±SEM; * represents P0.05, **P0.01. Source data are provided as a Source Data file.

FIGS. 7A-711: Mechanistic bases underlying sustained proliferation of DOT1L cKO cardiomyocytes. (FIG. 7A) Heatmap indicating the number of total and shared regions between differential H3K27ac ChIP-seq peaks, differential H3K79me2 ChIP-seq peaks and promoters (±200 bp around 5′TSS) in P1 CMs. Different intensities of gray scale indicate the fraction (%) of shared peaks. (FIGS. 7B-7C) UpSet plots indicating the number and percentage of genes down-(FIG. 7B) and upregulated (FIG. 7C) in P1 Dot1L cKO versus Ctrl CMs with or without H3K79me2 in gene body (GB) and/or regulatory elements (REs) and with and/or without differential H3K27ac REs. (FIG. 7D) Metascape pathway analysis of downregulated genes with H3K79me2 in GB and K79-REs (top) and upregulated genes without gene body H3K79me2 but with K79-REs (bottom) in Dot1L cKO vs Ctrl P1 CMs. Top 5 enriched categories are shown, sorted by Log₁₀ P value. (FIG. 7E) Browser tracks displaying H3K79me2 and H3K27ac ChIP-seq profiles of Ctrls (dark and light blue respectively) and Dot1L cKOs (red and orange respectively) P1 CMs in the genomic region harboring the Cdkn1b locus (encoding p27). Loops display all regulatory interactions between REs and the Cdkn1b gene, as identified by the ABC analysis. Gray loops identify interactions with REs without H3K79me2, light gray loops identify interactions with K79-REs, whereas dark gray loops represent interactions with K79-REs that additionally have H3K27ac REs differentially enriched between Dot1L Ctrls and cKOs. For reference, all REs are displayed in the middle lane in black or grey, regardless of their regulatory association with the Cdkn1b gene. Differential H3K27ac REs are indicated in the bottom lane in light gray when they overlap a K79-RE or dark gray when they do not. (FIG. 7F) Heatmap showing the expression of all transcription regulators downregulated in Dot1L cKO CMs at P1. (FIG. 7G) Motif enrichment analysis ranking transcription factors (TFs) enriched in REs associated with downregulated genes with H3K79me2 in GB and K79REs versus upregulated genes without H3K79me2 in GB and with K79REs. (FIG. 7H) Diagrammatic representation of mechanism of defective CM cell cycle withdrawal in the absence of DOT1L.

FIGS. 8A-8G: Patterns of Dot1l expression and strategy for its conditional deletion in cardiomyocytes from early embryonic timepoints. (FIGS. 8A-8B) RNA-scope detection of Dot1l transcripts in distinct embryonic stages. Dot1l was ubiquitously expressed in E10.5 embryos (FIG. 8A). In E16.5 heart, Dot1l mRNA could be detected in all cell types, but CMs (labeled by αSarcomeric Actinin in dark gray) displayed higher abundance of Dot1l transcripts than non-myocyte lineages (FIG. 8B) Scale bar=500 μm for low magnification panels and 50 μm for high magnification of boxed areas). (FIG. 8C) The xMlc2-Cre allele was highly specific and efficient in promoting recombination in embryonic CMs, as evidenced from the extensive overlap between the tdTomato recombination reporter signal (Cre+ cells, in light gray) and the CM marker Troponin T (TNNT, in medium scaled gray), as well as the lack of colocalization between tdTomato and the marker of mesenchymal lineages PDGFRα (white) (scale bar=500 μm low magnification top panels; 50 μm for high magnification of boxed areas). (FIG. 8D) FACS strategy to isolate highly pure populations of CMs based on the signal emitted by the red fluorescent protein tdTomato (expressed only in cells hit by the xMlc2-Cre). Negative selection for PDGFRα, CD31, TER119 and CD45 was used to exclude doublets with fibroblasts, endothelial cells, erythrocytes or leucocytes respectively. (FIG. 8E) Observed genotype distribution of live embryos recovered from mating between XMlc2-Cre; Dot1lΔ/+ and Dot1lfl/fl mice. No significant deviations from the expected Mendelian distribution (25% for each genotype) were observed until birth. (FIG. 8F) Graph representing a significant increase in heart weight (mg) in Dot1L cKOs vs Ctrls in all stages analyzed. (P1 Ctrl N=36, P1 cKO N=30, P5 Ctrl N=5, P5 cKO N=9, P10 Ctrl N=16, P10 cKO N=9 biological replicates; unpaired t-test, two-sided P1 P=0.0008, P10 P=<0.0001). (FIG. 8G) No significant body weight differences were observed between Ctrl and cKO mice at P1, P5 and P10. (P1 Ctrl N=36, P1 cKO N=30, P5 Ctrl N=5, P5 cKO N=9, P10 Ctrl N=16, P10 cKO N=9 biological replicates). Data is presented as mean±SEM; * represents P<0.05, **P0.01.

FIGS. 9A-9C: Absence of fibrotic remodeling in Dot1L cKO hearts. (FIG. 9A) qPCR for major Collagen genes (Col1a1 and Col3a1) typically upregulated in fibrotic responses revealed absence of significant differences between Dot1L Ctrl and cKO whole hearts at all stages analyzed (P1, P5 and P10) (N=3 biological replicates, data are presented as mean±SEM). (FIG. 9B) Immunostaining for Collagen1 (COL1, white) showed comparable results in hearts from Ctrls and cKOs, DAPI in dark gray and endogenous tdTomato signal driven by xMlc2-Cre; Rosa26-tdTomato in medium scaled gray. (Scale bar=50 μm). (FIG. 9C) Absence of fibrosis in Dot1L cKO vs Ctrl hearts was further confirmed by Masson trichrome staining at P1, P5 and P10. (Scale bar=1 mm for low magnification panels and 100 μm for high magnification panels).

FIGS. 10A-10B: Reduced Smyd1 expression in Dot1L cKO hearts. (FIG. 10A) Smyd1 expression is CM-restricted and can be observed in all chambers of control E10.5 hearts. At this stage, Smyd1 transcript levels were reduced both in the right and left ventricles of Dot1L cKO hearts, as shown in the higher magnification images of boxed areas (quantification data in FIG. 3I), confirming that DOT1L is necessary for normal Smyd1 expression. (FIG. 10B) A similar downregulation of Smyd1 transcript abundance was observed in both ventricles of E16.5 Dot1L cKO hearts. DAPI in darker gray, RNA-scope signal of Smyd1 probe in light gray and tdTomato signal driven by xMlc2-Cre; Rosa26-tdTomato in medium scale gray. To allow clear identification of RNA-scope signals, all images are shown both as a 2-color (Smyd1 and DAPI) and 3-color merge. (Scale bar=250 μm for low magnification panels and 50 μm for high magnification of boxed areas).

FIGS. 11A-11B: Normal expression of Hand2 in Dot1L cKO hearts. (FIG. 11A) In control E10.5 hearts, Hand2 transcripts can be detected in cardiomyocytes from all cardiac chambers and in the endocardium. As shown in the higher magnification of boxed areas, Hand2 RNA-scope signal was equally abundant in Ctrl and Dot1L cKO hearts (quantification data in FIG. 3F), confirming that expression of this gene is not affected by ablation of DOT1L. (FIG. 11B) At E16.5 Hand2 transcript levels were lower than those observed at E10.5. Similar to E10.5, no differences in Hand2 transcript abundance could be detected between genotype groups. DAPI in blue, RNA-scope signal of Hand2 probe in light gray and tdTomato signal driven by xMlc2-Cre; Rosa26-tdTomato in medium scale gray. To allow clear identification of RNA-scope signals, all images are shown in gray scale 2-color) (Hand2 and DAPI) and 3-color merge. (Scale bar=250 μm for low magnification panels and 50 μm for high magnification of boxed areas).

FIGS. 12A-12G: (FIGS. 12A-12B) Pie charts indicating the genomic distribution of H3K27ac ChIP-seq peaks (FIG. 12A) and H3K27ac/H3K79me2 shared ChIP-seq peaks (FIG. 12B) in E16.5 Ctrl CMs. (FIG. 12C) Table reporting how often, in E16.5 CMs, REs (top part) or interactions between a REs and its target gene (bottom part) overlap with promoters. All REs are shown on the left and K79-REs are shown on the right (overlap≥10%). Numbers do not include gonosomes. (FIGS. 12D-12F) Cumulative log₂FC distribution analyses in different categories of genes in E16.5 CMs: expression changes depending on the fraction of H3K79me2 coverage in the gene body (GB) (Ctrl coverage≥1 read) (FIG. 12D); expression changes depending on the number of K79-REs associated with the gene (FIG. 12E); expression changes between genes with gene body H3K79me2 and K79-REs versus genes with gene body H3K79me2 but no K79-REs (FIG. 12F). A Kolmogorov-Smirnov test was applied to assess statistical significance of differences between distribution of gene groups. (FIG. 12G) Browser tracks displaying H3K79me2 and H3K27ac ChIP-seq profiles of Ctrls (dark and light gray respectively) and Dot1L cKOs (dark and light gray respectively) E16.5 CMs in the genomic region harboring the Irx4 locus. Loops display all regulatory interactions between REs and the Irx4 gene, as identified by the ABC analysis. Gray loops identify interactions with REs without H3K79me2, light gray loops identify interactions with K79-REs, whereas dark gray loops represent interactions with K79-REs that additionally have H3K27ac REs differentially enriched between Dot1L Ctrls and cKOs. For reference, all REs are displayed in the middle lane in black or grey, regardless of their regulatory association with the Irx4 gene. Differential H3K27ac REs are indicated in the bottom lane in light gray when they overlap a K79-RE or dark gray when they do not.

FIGS. 13A-13G: Dot1L cKO cardiomyocytes fail to undergo neonatal cell cycle withdrawal. (FIG. 13A) Quantification of CM cell cycle phase distribution across the distinct cardiac chambers in histological sections from P10 Dot1L Ctrl and cKO hearts on a Rosa26-Fucci2A background (Cre-dependent cell cycle indicator). Light gray only signal represents CMs in G1; medium scaled gray signal represents CMs in G1/S; dark gray only signal represents CMs in S/G2/M. Right ventricle (RV), septum (Sept), left ventricle (LV), right atrium (RA) and left atrium (LA). Increased ratios of proliferative CMs (G1/S and S/G2/M) were found in all compartments of the Dot1L cKO heart (N=3 biological replicates) (FIG. 13B) Quantification of mitotic CMs (pH3+) in histological sections of P10 Dot1L Ctrl and cKO hearts (Rosa26-Fucci2A background). Compartment-specific data are presented as percentage of pH3+ CMs over total CMs (N=3 biological replicates; mean±SEM, unpaired t-test, two-sided RV P=0.0038). (FIG. 13C) Distribution of pH3+ cells according to the cell cycle stage indicated by the Fucci2A reporter. In both genotype groups the majority of pH3+ CMs corresponded to CMs in S/G2/M (light gray only) (N=3 biological replicates). (FIGS. 13D-13F) Immunofluorescence images (FIG. 13D) and corresponding quantification (FIG. 13E, FIG. 13F) showing signals of EdU incorporation (white) on histological sections from P10 Dot1L Ctrl and cKO hearts on a Rosa26-Fucci2A indicator background (Scale bars=50 μm). At P10, when analyzing all cardiac chambers together (FIG. 13E) (N=3 biological replicates; unpaired t-test, two-sided P=0.0011), or distinct compartments separately (FIG. 13F), Dot1L cKO hearts displayed significantly higher percentages of EdU+ CMs than their Ctrl counterparts (mean of 1022 CMs counted per heart; N=3 biological replicates; unpaired t-test, two-sided RV P=0.0175, LV P=0.0277, RA P=0.0134). (FIG. 13G) Distribution of EdU+ cells according to the cell cycle stage indicated by the Fucci2A reporter (N=3 biological replicates). In both genotype groups all EdU+ CMs corresponded to CMs in G1/S (medium scaled gray) or S/G2/M (light gray only). * represents P≤0.05 and ** P≤0.01; Data are presented as mean±SEM.

FIGS. 14A-14M: Mechanisms of gene expression regulation by DOT1L/H3K79me2 in postnatal day 1 cardiomyocytes. (FIG. 14A) Volcano plot displaying H3K79me2 ChIP-seq peaks significantly enriched in P1 cKO vs Ctrl CMs. (FIG. 14B) Pie chart indicating the genomic distribution of differential H3K79me2 ChIP-seq peaks in P1 CMs. (FIG. 14C) Metagene profiles showing the average distribution of H3K79me2 input-normalized density relative to Transcription Start Site (TSS) and Transcription Termination Site (TTS) with ±2 Kb flanking regions. (FIG. 14D) Fraction of gene body covered with H3K79me2 in downregulated genes (left graph) and in upregulated genes (right graph) (FIG. 14E) Graph representing the percentage of down- and up-regulated genes in P1 cKO CMs with (Coverage≥50 reads and Fraction of gene body≥0.2) or without (Coverage<50 reads or Fraction of gene body<0.2) gene body H3K79me2 in P1 control CMs. (FIG. 14F) Percentage of genes with H3K79me2 in the gene body (GB) or without H3K79me2 in the gene body across distinct expression quartiles. (FIG. 14G) Pie chart indicating the genomic distribution of H3K27ac ChIP-seq peaks in P1 Ctrl CMs. (FIGS. 14H-14J) Cumulative log2FC distribution analyses in different categories of genes in P1 CMs: expression changes depending on the fraction of H3K79me2 coverage in the gene body (GB) (Ctrl coverage≥1 read) (FIG. 14D); expression changes depending on the number of K79-REs associated with the gene (FIG. 14E); expression changes between genes with gene body H3K79me2 and K79-REs versus genes with gene body H3K79me2 but no K79-REs (FIG. 14F). A Kolmogorov-Smirnov test was applied to assess statistical significance of differences between distribution of gene groups. (FIG. 14K) Volcano plot displaying H3K27ac ChIP-seq peaks significantly enriched in P1 cKO vs Ctrl CMs. (FIG. 14L) Graph representing the percentage of differential H3K27ac ChIP-seq peaks in P1 cKO vs Ctrl CMs overlapping (orange) or not overlapping (grey) with H3K79me2 ChIP-seq peaks. (oddsratio=0.006; p-value=0). (FIG. 14M) Table reporting how often, in P1 CMs, REs (top part) or interactions between a REs and its target gene (bottom part) overlap with promoters. All REs are shown on the left and K79-REs on the right (overlap≥10%). Numbers do not include gonosomes.

FIG. 15: Upregulation of neuronal genes in Dot1L cKO hearts in early cardiogenesis. qPCR analyses showing that multiple of the neuronal genes upregulated in P1 Dot1L cKO vs Ctrl CMs are already upregulated in early cardiogenesis (E10.5 hearts). (N=4 biological replicates, Data is presented as mean±SEM; unpaired t-test, two-sided Gpc2 P=0.033, Nefl P=0.009, Nefm P=0.022, Ntng2 P=0.028, Gabrrl P=0.006, Cadps2 P=0.04, Erc2 P=0.006, Sh3gl2 P=0.009; * represents P0.05, **P<0.01).

FIGS. 16A-16H: DOT1L ablation in adult cardiomyocytes results in cardiac enlargement and lethality at late timepoints post-tamoxifen injection. (FIG. 16A) diagram of the experimental design used for ablation of Dot1L in murine adult cardiomyocytes. (FIG. 16B) qPCR showing efficient loss of Dot1l mRNA in iKO animals. (FIG. 16C) Western blot showing loss of H3K79me2 in iKOs. (FIG. 16D) Kaplan-Meier survival plot of iKOs and control littermates. (FIG. 16E) Whole heart images detecting the signal of the cardiomyocyte-specific red fluorescent protein tdTomato, showing enlargement of iKOs at 4- and 8-month post-tamoxifen. (FIG. 16F) In comparison with controls, iKOs had increased heart weight/body weight ratios. (FIG. 16G) In comparison with controls, iKOs had increased heart weight. (FIG. 16H) Controls and iKOs had had similar body weight.

FIGS. 17A-17B: A biphasic phenotype resulting from DOT1L ablation in adult cardiomyocytes. (FIG. 17A) echocardiographic time course analyses showed that iKO animals had heavier hearts as early as 2-month post-tamoxifen. In later time points (4- and 8-month post-tamoxifen) iKOs also showed reduced cardiac function. (FIG. 17B) histological timecourse (trichrome staining) showing two distinct iKO phenotypes in early versus late timepoints: increased ventricular thickness (arrows) with normal cardiac morphology at 2-month post-tamoxifen, versus chamber dilation at 8-month post-tamoxifen.

FIGS. 18A-18D: Absence of fibrosis, vascular density or cardiomyocyte hypertrophy upon DOT1L ablation in adult cardiomyocytes. (FIG. 18A) Histological analyses showing that, at 2-month post-tamoxifen, iKOs were comparable to controls in terms of fibroblast abundance (PDGFRα+ cells), extracellular matrix content (Col1a1 staining) and vascular coverage (CD31+ cells). (FIGS. 18B-18D) at 2-month post-tamoxifen, iKOs also showed normal cardiomyocyte length (FIG. 18B), width (FIG. 18C) and area (FIG. 18D).

FIGS. 19A-19F: De novo proliferation of adult cardiomyocytes upon DOT1L ablation. (FIG. 19A) Histological analyses assessing cells engaged in DNA synthesis (EdU+) in control and iKO hearts 2-month post-tamoxifen. In controls all EdU+ cells were tdTomato-(therefore non-cardiomyocytes), whereas in iKOs abundant EdU+ cardiomyocytes (EdU+, tdTomato) could be detected. (FIG. 19B) Cardiomyocytes isolated from iKO hearts at 2-month post-tamoxifen were often EdU+, whereas cardiomyocytes from controls were not. (FIG. 19C) Quantification of the rate of EdU+ cardiomyocytes in controls and iKOs at 2-month post-tamoxifen. (FIG. 19D) At 2-month post-tamoxifen, controls and iKOs had similar nucleation profiles. (FIG. 19E) 2-month post-tamoxifen, EdU+ cardiomyocytes in iKOs were more often mononucleated than EdU-cardiomyocytes, showing that DNA synthesis was not a consequence of increased binucleation. (FIG. 19F) 2-month post-tamoxifen, iKO hearts had cardiomyocytes positive for the mitotic marker phospho-histone3 (pH3) and showed DNA patterns (DAPI staining) characteristic of mitotic cells.

FIGS. 20A-20B: Transcriptomic changes underlying de novo adult cardiomyocyte proliferation upon DOT1L ablation. (FIG. 20A) Functional annotation of genes upregulated in iKO cardiomyocytes at 2-month post-tamoxifen, showing a very significant enrichment in transcripts associated with cell cycle. (FIG. 20B) Functional annotation of genes downregulated in iKO cardiomyocytes at 2-month post-tamoxifen, showing a very significant enrichment in transcripts associated with maturation and metabolism of differentiated cardiomyocytes.

### DETAILED DESCRIPTION OF THE DISCLOSURE

### Definitions

As it would be understood, the section or subsection headings as used herein is for organizational purposes only and are not to be construed as limiting or separating or both limiting and separating the subject matter described.

Throughout this disclosure, various publications, patents and published patent specifications are referenced by an identifying citation. The disclosures of these publications, patents and published patent specifications are hereby incorporated by reference into the present disclosure in their entireties to more fully describe the state of the art to which this invention pertains.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compounds, compositions and methods include the recited elements, but not exclude others. “Consisting essentially of” when used to define compounds, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” It also is to be understood, although not always explicitly stated, that the reagents described herein are merely exemplary and that equivalents of such are known in the art.

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) 15%, 10%, 5%, 3%, 2%, or 1%.

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

As used herein, the term “animal” refers to living multi-cellular vertebrate organisms, a category that includes, for example, mammals and birds. The term “mammal” includes both human and non-human mammals.

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human.

A “composition” as used herein, refers to an active agent, such as a compound as disclosed herein and a carrier, inert or active. The carrier can be, without limitation, solid such as a bead or resin, or liquid, such as phosphate buffered saline.

Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. In general, one will desire to administer an amount of the compound that is effective to achieve a serum level commensurate with the concentrations found to be effective in vivo. These considerations, as well as effective formulations and administration procedures are well known in the art and are described in standard textbooks.

As used herein, the term “modulate” means to modify the normal homeostasis of a cell or tissue or to effect a systemic variation in a genotypic or phenotypic characteristic of a cell or tissue. An agent that modulates a cell or tissue can upregulate or increase gene expression or function. Alternatively, an agent can modulate a cell or tissue by downregulating or abolishing gene expression or function. Modulation can occur in vitro or in vivo.

As used herein the term “abrogates” intends to negate or nullify.

As used herein, the term “cardiac regeneration” intends reversing or repairing damaged heart cells or tissue.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, treatment excludes prophylaxis.

The terms “oligonucleotide” or “polynucleotide” or “portion,” or “segment” thereof refer to a stretch of polynucleotide residues which is long enough to use in PCR or various hybridization procedures to identify or amplify identical or related parts of mRNA or DNA molecules. The polynucleotide compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

As used herein, an amino acid (aa) or nucleotide (nt) residue position in a sequence of interest “corresponding to” an identified position in a reference sequence refers to that the residue position is aligned to the identified position in a sequence alignment between the sequence of interest and the reference sequence. Various programs are available for performing such sequence alignments, such as Clustal Omega and BLAST. In one aspect, equivalent polynucleotides, proteins and corresponding sequences can be determined using BLAST (accessible at blast.ncbi.nlm.nih.gov/Blast.cgi, last accessed on Aug. 1, 2021).

It is to be inferred without explicit recitation and unless otherwise intended, that when the present disclosure relates to a polypeptide, amino acid sequence, protein, polynucleotide, an equivalent or a biologically equivalent of such is intended within the scope of this disclosure. As used herein, the term “biological equivalent thereof” is intended to be synonymous with “equivalent thereof” when referring to a reference protein, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity, or at least about 85% homology or identity, or alternatively at least about 90% homology or identity, or alternatively at least about 95% homology or identity, or alternatively at least about 96% homology or identity, or alternatively at least about 97% homology or identity, or alternatively at least about 98% homology or identity, or alternatively at least about 99% homology or identity (in one aspect, as determined using the Clustal Omega alignment program) and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complementary sequence.

In some embodiments, a first sequence (nucleic acid sequence or amino acid) is compared to a second sequence, and the identity percentage between the two sequences can be calculated. In further embodiments, the first sequence can be referred to herein as an equivalent and the second sequence can be referred to herein as a reference sequence. In yet further embodiments, the identity percentage is calculated based on the full-length sequence of the first sequence. In other embodiments, the identity percentage is calculated based on the full-length sequence of the second sequence.

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

As used herein, the term “animal” refers to living multi-cellular vertebrate organisms, a category that includes, for example, mammals and birds. The term “mammal” includes both human and non-human mammals.

A cardiomyocyte (CM) is a muscle cell that forms the chambers of the heart. They are usually divided into two types of cells, the pacemaker cells and the force-producing ventricular and atrial CMs. See, Talman and Kivela, Front. Cardiovasc. Med. (2018) Jul. 26, 2018, https://www.frontiersin.org/article/10.3389/fcvm.2018.00101.

In some aspects, a “cardiomyocyte” or “cardiac myocyte” is a specialized muscle cell which primarily forms the myocardium of the heart. Cardiomyocytes have five major components: 1. cell membrane (sarcolemma) and T-tubules, for impulse conduction, 2. sarcoplasmic reticulum, a calcium reservoir needed for contraction, 3. contractile elements, 4. mitochondria, and 5. a nucleus. Cardiomyocytes can be subdivided into subtypes including, but not limited to, atrial cardiomyocyte, ventricular cardiomyocyte, SA nodal cardiomyocyte, peripheral SA nodal cardiomyocyte, or central SA nodal cardiomyocyte. Stem cells can be propagated to mimic the physiological functions of cardiomyocytes or alternatively, differentiate into cardiomyocytes. This differentiation can be detected by the use of markers selected from, but not limited to, myosin heavy chain, myosin light chain, actinin, troponin, tropomyosin, GATA4, Mef2c, and Nkx2.5.

The cardiomyocyte marker “myosin heavy chain” and “myosin light chain” are part of a large family of motor proteins found in muscle cells responsible for producing contractile force. These proteins have been sequenced and characterized, see for example GenBank Accession Nos. AAD29948, CAC70714, CAC70712, CAA29119, P12883, NP_000248, P13533, CAA37068, ABR18779, AAA59895, AAA59891, AAA59855, AAB91993, AAH31006, NP_000423, and ABC84220. The genes for these proteins has also been sequenced and characterized, see for example GenBank Accession Nos. NM_002472 and NM_000432.

The cardiomyocyte marker “actinin” is a microfilament protein which are the thinnest filaments of the cytoskeleton found in the cytoplasm of all eukaryotic cells. Actin polymers also play a role in actomyosin-driven contractile processes and serve as platforms for myosin's ATP hydrolysis-dependent pulling action in muscle contraction. This protein has been sequenced and characterized, see for example GenBank Accession Nos. NP_001093, NP_001095, NP 001094, NP_004915, P35609, NP_598917, NP 112267, AA107534, and NP_001029807. The gene for this protein has also been sequenced and characterized, see for example GenBank Accession Nos. NM_001102, NM_004924, and NM_001103.

The cardiomyocyte marker “troponin” is a complex of three proteins that is integral to muscle contraction in skeletal and cardiac muscle. Troponin is attached to the protein “tropomyosin” and lies within the groove between actin filaments in muscle tissue. Tropomyosin can be used as a cardiomyocyte marker. These proteins have been sequenced and characterized, see for example GenBank Accession Nos. NP_000354, NP_003272, P19429, NP_001001430, AAB59509, AAA36771, and NP_001018007. The gene for this protein has also been sequenced and characterized, see for example GenBank Accession Nos. NM_000363, NM_152263, and NM_001018007. A “cardiac stem cell” is an adult tissue-resident cell with the capacity to differentiate into cardiomvocytes, and vascular endothelial cells and smooth muscle cells. When activated, CSCs proliferate and form lineage-committed progenitors, which will progressively acquire a fully mature phenotype, forming new contractile muscle and blood vessels for tissue oxygenation. Identifying cell markers have been reported, e.g., c-kit, stem cell antigen-1 (Sca-1) and PECAM-1, or CD31 See e.g., Goichberg et al. (2014) Antioxid. Redox Signal, Nv. 10; 21(14): 2002-2017.DOT1L is a histone methyltransferase that methylates lysine-79 of histone H3 found in humans and other eukaryotes. An exemplary human protein sequence is disclosed at NP_115871 (last accessed on Apr. 19, 2022) and reproduced below:

| | |
|---|---|
| 1 | mgeklelrlk spvgaepavy pwplpvydkh hdaaheiiet irwvceeipd lklamenyvl |
| 61 | idydtksfes mqrlcdkynr aidsihqlwk gttqpmklnt rpstgllrhi lqqvynhsvt |
| 121 | dpeklnnyep fspevygets fdlvaqmide ikmtdddlfv dlgsgvgqvv lqvaaatnck |
| 181 | hhygvekadi pakyaetmdr efrkwmkwyg kkhaeytler gdflseewre riantsvifv |
| 241 | nnfafgpevd hqlkerfanm keggrivssk pfaplnfrin srnlsdigti mrvvelsplk |
| 301 | gsvswtgkpv syylhtidrt ilenyfsslk npklreeqea arrrqqresk snaatptkgp |
| 361 | egkvagpada pmdsgaeeek agaatvkkps pskarkkkln kkgrkmagrk rgrpkkmnta |
| 421 | nperkpkknq taldalhaqt vsqtaasspq dayrsphspf yqlppsvqrh spnpllvapt |
| 481 | ppalqklles fkiqylqfla ytktpqykas lqellgqeke knaqllgaaq qllshcqaqk |
| 541 | eeirrlfqqk ldelgvkalt yndliqaqke isahnqqlre qseqleqdnr alrgqslqll |
| 601 | karceelqld watlslekll kekqalksqi sekqrhclel qisiveleks qrqqellqlk |
| 661 | scvppddals lhlrgkgalg relepdasrl hleldctkfs lphlssmspe lsmngqaagy |
| 721 | elcgvlsrps skqntpqyla spldqevvpc tpshvgrprl eklsglaapd ytrlspakiv |
| 781 | lrrhlsqdht vpgrpaasel hsraehtken glpyqspsvp gsmklspqdp rplspgalql |
| 841 | agekssekgl reraygssge litslpisip lstvqpnklp vsiplasvvl psraerarst |
| 901 | pspvlqprdp sstlekqiga nahgagsrsl alapagfsya gsvaisgala gspasltpga |
| 961 | epatldesss sgslfatvgs rsstpqhpll laqprnslpa spahqlsssp rlggaaqgpl |
| 1021 | peaskgdlps dsgfsdpese akrrivftit tgagsakqsp sskhspltas argdcvpshg |
| 1081 | qdsrrrgrrk rasagtpsls agvspkrral psvaglftqp sgsplnlnsm vsninqplei |
| 1141 | taisspetsl ksspvpyqdh dqppvlkker plsqtngahy spltsdeepg sedepssari |
| 1201 | erkiatisle sksppktlen ggglagrkpa pagepvnssk wkstfspisd iglaksadsp |
| 1261 | lqassalsqn slftfrpale epsadaklaa hprkgfpgsl sgadglspgt npangctfgg |
| 1321 | glaadlslhs fsdgaslphk gpeaaglssp lsfpsqrgke gsdanpflsk rqldglaglk |
| 1381 | gegsrgkeag egglplcgpt dktpllsgka akardrevdl knghnlfisa aavppgslls |
| 1441 | gpglapaass aggaassaqt hrsflgpfpp gpqfalgpms lqanlgsvag ssvlqslfss |
| 1501 | vpaaaglvhv ssaatrltns hamgsfsgva ggtvggn |

An exemplary polynucleotide encoding a human DOT1L protein is found at NM_032482.3 (last accessed on Apr. 19, 2022):

| | |
|---|---|
| 1 | attgtgctcg cttcacgccg gcccaagatg gcggaggcgc tggaggcccc gggcctgtga |
| 61 | ctacaaagag ggagtcgggg gccgggccgg accggagcgc ggcggcggcg gcggcggcgg |
| 121 | ccgaggccga ggccaggccc cctcccctca gcctcccgcc cctccctccc gcccgccctc |
| 181 | ctccgcccac cggcggcccc gcccctcccc caaccgcccg cctagcatgg tgcggcggcc |
| 241 | gcgcgcgcgg acatggggga gaagctggag ctgagactga agtcgcccgt gggggctgag |
| 301 | cccgccgtct acccgtggcc gctgccggtc tacgataaac atcacgatgc tgctcatgaa |
| 361 | atcatcgaga ccatccgatg ggtctgtgaa gaaatcccgg atctcaagct cgctatggag |
| 421 | aattacgttt taattgacta tgacaccaaa agcttcgaga gcatgcagag gctctgcgac |
| 481 | aagtacaacc gtgccatcga cagcatccac cagctgtgga agggcaccac gcagcccatg |
| 541 | aagctgaaca cgcggccgtc cactggactc ctgcgccata tcctgcagca ggtctacaac |
| 601 | cactcggtga ccgaccccga gaagctcaac aactacgagc ccttctcccc cgaggtgtac |
| 661 | ggggagacct ccttcgacct ggtggcccag atgattgatg agatcaagat gaccgacgac |
| 721 | gacctgtttg tggacttggg gagcggtgtg ggccaggtcg tgctccaggt tgctgctgcc |
| 781 | accaactgca aacatcacta tggcgtcgag aaagcagaca tcccggccaa gtatgcggag |
| 841 | accatggacc gcgagttcag gaagtggatg aaatggtatg gaaaaaagca tgcagaatac |
| 901 | acattggaga gaggcgattt cctctcagaa gagtggaggg agcgaatcgc caacacgagt |
| 961 | gttatatttg tgaataattt tgcctttggt cctgaggtgg atcaccagct gaaggagcgg |
| 1021 | tttgcaaaca tgaaggaagg tggcagaatc gtgtcctcga aaccctttgc acctctgaac |
| 1081 | ttcagaataa acagtagaaa cttgagtgac atcggcacca tcatgcgcgt ggtggagctc |
| 1141 | tcgcccctga agggctcggt gtcgtggacg gggaagccag tctcctacta cctgcacact |
| 1201 | atcgaccgca ccatacttga aaactatttt tctagtctga aaaacccaaa actcagggag |
| 1261 | gaacaggagg cagcccggcg ccgccagcag cgcgagagca agagcaacgc ggccacgccc |
| 1321 | actaagggcc cagagggcaa ggtggccggc cccgccgacg cccccatgga ctctggtgct |
| 1381 | gaggaagaga aggcgggagc agccaccgtg aagaagccgt ctccctccaa agcccgcaag |
| 1441 | aagaagctaa acaagaaggg gaggaagatg gctggccgca agcgcgggcg ccccaagaag |
| 1501 | atgaacactg cgaaccccga gcggaagccc aagaagaacc aaactgcact ggatgccctg |
| 1561 | cacgctcaga ccgtgtctca gacggcggcc tcctcacccc aggatgccta cagatcccct |
| 1621 | cacagcccgt tctaccagct acctccgagc gtgcagcggc actcccccaa cccgctgctg |
| 1681 | gtggcgccca ccccgcccgc gctgcagaag cttctagagt ccttcaagat ccagtacctg |
| 1741 | cagttcctgg catacacaaa gaccccccag tacaaggcca gcctgcagga gctgctgggc |
| 1801 | caggagaagg agaagaacgc ccagctcctg ggtgcggctc agcagctcct cagccactgc |
| 1861 | caggcccaga aggaggagat caggaggctg tttcagcaaa aattggatga gctgggtgtg |
| 1921 | aaggcgctga cctacaacga cctgattcaa gcgcagaagg agatctccgc ccataaccag |
| 1981 | cagctgcggg agcagtcgga gcagctggag caggacaacc gcgcgctccg cggccagagc |
| 2041 | ttgcagctgc tcaaggctcg ctgcgaggag ctgcagctgg actgggccac gctgtcgctg |
| 2101 | gagaagctgt tgaaggagaa gcaggccctg aagagccaga tctcggagaa gcagaggcac |
| 2161 | tgcctggagc tgcagatcag cattgtggag ctagagaaga gccagcggca gcaggagctc |
| 2221 | ctgcagctca agtcctgtgt gccgcctgac gacgccctgt ccctgcacct gcgtgggaag |
| 2281 | ggcgccctgg gccgcgagct ggagcctgac gccagccggc tgcacctgga gctggactgc |
| 2341 | accaagttct cgctgcctca cttgagcagc atgagcccgg agctctccat gaacggccag |
| 2401 | gctgctggct atgagctctg cggtgtgctg agccggcctt cgtcgaagca gaacacgccc |
| 2461 | cagtacctgg cctcacccct ggaccaggag gtggtgccct gtacccctag ccacgtcggc |
| 2521 | cggccgcgcc tggagaagct gtctggccta gccgcacccg actacactag gctgtccccg |
| 2581 | gccaagattg tgctgaggcg gcacctgagc caggaccaca cggtgcccgg caggccggct |
| 2641 | gccagtgagc tgcattcgag agctgagcac accaaggaga acggccttcc ctaccagagc |
| 2701 | cccagcgtgc ctggcagcat gaagctgagc cctcaggacc cgcggcccct gtcccctggg |
| 2761 | gccttgcagc ttgctggaga gaagagcagt gagaagggcc tgagagagcg cgcctacggc |
| 2821 | agcagcgggg agctcatcac cagcctgccc atcagcatcc cgctcagcac cgtgcagccc |
| 2881 | aacaagctcc cggtcagcat tcccctggcc agcgtggtgc tgcccagccg cgccgagagg |
| 2941 | gcgaggagca cccccagtcc cgtgctgcag ccccgtgacc cctcgtccac acttgaaaag |
| 3001 | cagattggtg ctaatgccca cggtgctggg agcagaagcc ttgccctggc ccccgcaggc |
| 3061 | ttctcctacg ctggctcggt ggccatcagc ggggccttgg cgggcagccc ggcctctctc |
| 3121 | acacctggag ccgagccggc caccttggat gagtcctcca gctctgggag cctttttgcc |
| 3181 | accgtggggt cccgcagctc cacgccacag caccccctgc tgctggcaca gccccggaac |
| 3241 | tcgcttcctg cctctcccgc ccaccagctc tcctccagtc cccggcttgg tggggccgcc |
| 3301 | cagggcccgt tgcccgaggc cagcaaggga gacctgccct ccgattccgg cttctcagat |
| 3361 | cctgagagtg aagccaagag gaggattgtg ttcaccatca ccactggtgc gggcagtgcc |
| 3421 | aagcagtcgc cctccagcaa gcacagcccc ctgaccgcca gcgcccgtgg ggactgtgtg |
| 3481 | ccgagccacg ggcaggacag tcgcaggcgc ggccggcgga agcgagcatc tgcggggacg |
| 3541 | cccagcttga gcgcaggcgt gtcccccaag cgccgagccc tgccgtccgt cgctggcctt |
| 3601 | ttcacacagc cttcggggtc tcccctcaac ctcaactcca tggtcagtaa catcaaccag |
| 3661 | cccctggaga ttacagccat ctcgtccccg gagacctccc tgaagagctc ccctgtgccc |
| 3721 | taccaggacc acgaccagcc ccccgtgctc aagaaggagc ggcctctgag ccagaccaat |
| 3781 | ggggcacact actccccact cacctcagac gaggagccag gctctgagga cgagcccagc |
| 3841 | agtgctcgaa ttgagagaaa aattgcaaca atctccttag aaagcaaatc tcccccgaaa |
| 3901 | accttggaaa atggtggtgg cttggcggga aggaagcccg cgcccgccgg cgagccagtc |
| 3961 | aatagcagca agtggaagtc caccttctcg cccatctccg acatcggcct ggccaagtcg |
| 4021 | gcggacagcc cgctgcaggc cagctccgcc ctcagccaga actccctgtt cacgttccgg |
| 4081 | cccgccctgg aggagccctc tgccgatgcc aagctggccg ctcaccccag gaaaggcttt |
| 4141 | cccggctccc tgtcgggggc tgacggactc agcccgggca ccaaccctgc caacggctgc |
| 4201 | accttcggcg ggggcctggc cgcggacctg agtttacaca gcttcagtga tggtgcttct |
| 4261 | cttccccaca agggccccga ggcggccggc ctgagctccc cgctgagctt cccctcgcag |
| 4321 | cgcggcaagg agggctcgga cgccaaccct ttcctgagca agaggcagct ggacggcctg |
| 4381 | gctgggctga agggcgaggg cagccgcggc aaggaggcag gggagggcgg cctaccgctg |
| 4441 | tgcgggccca cggacaagac cccactgctg agcggcaagg ccgccaaggc ccgggaccgc |
| 4501 | gaggtcgacc tcaagaatgg ccacaacctc ttcatctctg cggcggccgt gcctcccgga |
| 4561 | agcctcctca gcggccccgg cctggccccg gcggcgtcct ccgcaggcgg cgcggcgtcc |
| 4621 | tccgcccaga cgcaccggtc cttcctgggc cccttcccgc cgggaccgca gttcgcgctc |
| 4681 | ggccccatgt ccctgcaggc caacctcggc tccgtggccg gctcctccgt gctgcagtcg |
| 4741 | ctgttcagct ctgtgccggc cgccgcaggc ctggtgcacg tgtcgtccgc tgccaccaga |
| 4801 | ctgaccaact cgcacgccat gggcagcttt tccggggtgg caggcggcac agttggaggt |
| 4861 | aactaggatt tctacctcaa ccgcgagacc tatgcaagga cggtgtggac caactcgcgc |
| 4921 | ccgcggcatg gtgcccgccg gcctgccggg ctcccacccc tggacggcag aggcaaggac |
| 4981 | ggacgggagc tccactgtga atcggcggca cgcgccgcag gaggctggga ctggtccagt |
| 5041 | ttgtactgtc gatagtttta gataaagtat ttatcatttt ttaaaaagta taaacaattc |
| 5101 | tgacttattt tattccatct aagtggtaaa aggcaactta ttgagaaata taaatatcta |
| 5161 | tatatgagag ctctatataa agacacgtgt ctgcagggcg ggcccgccag cggattcgcc |
| 5221 | acagcctgcc ccggtgctat ctcgtcccca ggcccgcgcc tgcctccacc cgcttggtgc |
| 5281 | tgactagacg ctgacaacgc cgaaccccgt tctcggaaac gccgcccggc cggctccccc |
| 5341 | gacgcgctgc tcccgtacca aaggcaggcc cgtcgccacc acattcctcg gaggcctccc |
| 5401 | cgcggcctga gccccttcct gagcgccctg gcgcctgccc tgagctcttc acctttaccc |
| 5461 | cggcactgtg aacccccaga ctgttcaccc tccggggcgt gggttgcgcc cttgcatgtg |
| 5521 | aaggggcctg cgcggtgacg cagctggcca tgtgctgcgc gatggtgctg tgaggacggc |
| 5581 | gcgggcacgt tgaacaagtg catttacttt tgtatttctc ggctgtccat ggctcgcagc |
| 5641 | atgccctgcg atgcggggca ggcctgtcgt gggtcccttg gtgtttctgt acaggagaga |
| 5701 | gtcacactaa tgagtggcag tattttatag agatgtgatg agaatttata aatttcatag |
| 5761 | atttgacagc ttttattttt agatggtata atgcacagtg aagaggaaag aaaagcgagg |
| 5821 | ggaaaaaacc ttatttattc aaacagtgca caaaatggcc ccagcgtcag ccccgaccct |
| 5881 | agacccctca gttgcagctc ccagcagccc agacagagct gccggcgccc ctgcctgccc |
| 5941 | cacatccctt cctgtcaggg ccacgcctgg cacccatccc ttggagcctg tgctggttct |
| 6001 | cccagctgct gtgggtgtgc tggggccagg gtgcactgct gaaacctggc ctctctggcc |
| 6061 | ctaggcccca gggtgacgtc ggccccccac tctgcagcct tggcgggtgc ctgggactgg |
| 6121 | gtgtggaagg agaggagctg aggccggggt gtagcaggca ggcagggcca ctccagtgct |
| 6181 | tctggagccc tgagcagtca gggcctgggt tgtctgagca gtggtggctc tgtgccctcc |
| 6241 | ctggaggatg ggatctggga gtctgagctc cccgcatctg gccctgggct gtgtggcact |
| 6301 | tgctgagccc accttctcaa gtgcttgctc ctgtgagatg gcatcgggga gccccttccc |
| 6361 | caaggtgcca cagatccacc ctccagggag ctgccagccc tgtgttctgg ttcccaaggg |
| 6421 | caggatggac acacgtcaca tccctaccac gtggcctcca aagggagcca cggaggaaag |
| 6481 | gcttctgtgg ttgctaggtg ggggagtcct gtgtgggagg gcctgaagac ccctgcttgt |
| 6541 | gcctggtgag gggggtgctg cctcccccag cccccaacaa cctctcagac ccccaccctc |
| 6601 | caacatagct gagttctgaa gatggtgctc cggacctgtc ctcttaagtg gtgcccagtg |
| 6661 | ccctccccac cccacgttgg tgctctcagc tagaaggtgc tgtgcctctg cctgagcccc |
| 6721 | aagccccgag cctggccttc aggacaggca gcctgctctg tgtcgccacg ggccggatac |
| 6781 | gccacagggt tgatggcaga gacggccgag tccctggtct agaacaagac acattcttta |
| 6841 | aacactgtat tacttctgcc tccctctagg tgacagtggc agtccgggtg ccatcacggg |
| 6901 | tcctgcagat ggccatgcag ggctcctgcc cacgcaggcc accgtatgtt caggacacgc |
| 6961 | actgggtctc agagccactg gcccaggcag aagtctcctt gagcccactg ggtcatatgc |
| 7021 | gtgtcaccac acgtgaacta gtgtggtggc tgcctgcgga caccctcctg ttctgagccc |
| 7081 | tgggcctgtg ttcttctcag acactcccag actgaggggt ggtgtgtggc gggtggcagg |
| 7141 | gtggctgtgg agactgggga tctggagcct ggtgctggca cctggcctga gtttccgtgg |
| 7201 | gcagctggcg gggacctgtg ctgctgctgc tgactgtggg tgggcgggcg gcgcctggga |
| 7261 | gtggctcttg ctcaggaatt gataggaacc ctaaaaacta ggataccccc tcctcggccc |
| 7321 | atgaggcacg cacagtgact tatttaagac ttccccctta atttatctgc ccccaggatg |
| 7381 | cgtcagtctg ttcagtggtc agcaggcccc ccaccccccg ccgactgccc tcgccatcgt |
| 7441 | ggtcagaccc ccctcccaac acaacacgct gctggtctgt gtcagccttt gtaacgtggg |
| 7501 | aggctctgcc gtgtcttccg ggtgaactgt atttggattg cgcgcattgt cacggtccgc |
| 7561 | ccctgggctg caggcgcccc ttcctctggg cacccctgca ttctgcatcc ccacctctag |
| 7621 | acgctgtaat aaacagactg ttttcactcg ga |

A DOT1L inhibitor is an agent that inhibits, reduces or abrogates the biological activity of DOT1L. Some are known in the art and under the development for the treatment of leukemia. See, e.g., Perner et al. (2020) Novel inhibitors of the histone methyltransferase DOT1L show potent antileukemic activity in patient derived xenografts, Blood 136(17):1983-1988.

EPZ004777 is a potent, selective DOT1L inhibitor with IC50 of 0.4 nM in a cell-free assay. It is available from Selleckchem.com (https://www.selleckchem.com/products/epz004777.html) and has the structure:

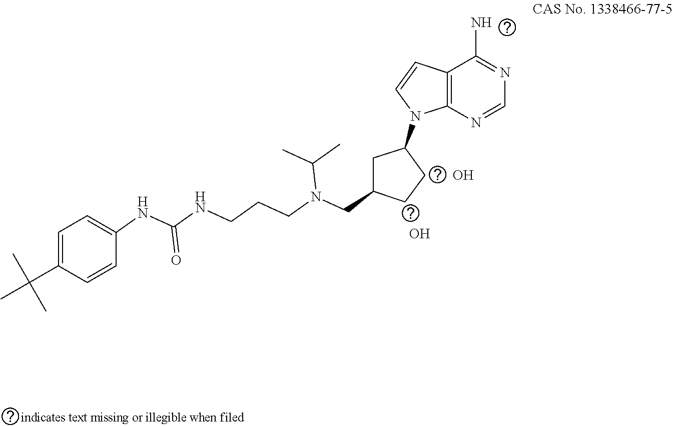

SGC0946 is a potent selective DOT1L inhibitor that is commercially available from ApexBio (http://www.apexbt.com/sgc-0946.html?gclid=EAIaIQobChMIqfTaraCt_gIVnyutBh1kgAfTEAAYASAAEgJhOfD_BwE

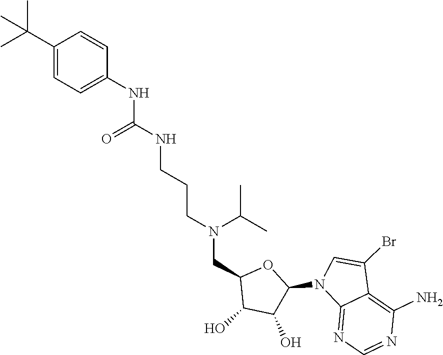 [SMILES: CC(C)N(CCCNC(=O)Nc1ccc(C(C)(C)C)cc1)C[C@H]1O[C@@H](n2cc(Br)c3c(N)ncnc32)[C@H](O)[C@@H]1O]

EPZ-5676 is a potent inhibitor of DOT1L and is commercially available from ApexBio (https://www.apexbt.com/epz5676.html?gclid=EAIaIQobChMI6PHKr6Gt gIVOhR9Ch1Do Q6-EAAYASAAEgKbsfD_BwE). EPZ-5675 can be delivered via daily intraperitoneal injection at a dose of 20/mg/kg/day. The chemical structure is:

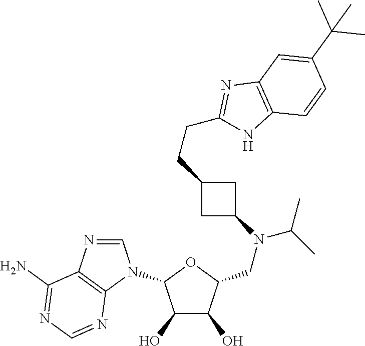 [SMILES: CC(C)N(C[C@H]1O[C@@H](n2cnc3c(N)ncnc32)[C@H](O)[C@@H]1O)[C@H]1C[C@@H](CCc2nc3cc(C(C)(C)C)ccc3[nH]2)C1]

A “gene delivery vehicle” is defined as any molecule that can carry inserted polynucleotides into a host cell. Examples of gene delivery vehicles are liposomes, biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus, adeno-associated virus, and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection, sometimes called transduction), transfection, transformation or various other protein-based or lipid-based gene delivery complexes) as well as techniques facilitating the delivery of “naked” polynucleotides (such as electroporation, “gene gun” delivery and various other techniques used for the introduction of polynucleotides). Unless otherwise specified, the term transfected, transduced or transformed may be used interchangeably herein to indicate the presence of exogenous polynucleotides or the expressed polypeptide therefrom in a cell. The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

The term “express” refers to the production of a gene product. In some embodiments, the gene product is a polypeptide or protein. In some embodiments, the gene product is an mRNA, a tRNA, an rRNA, a miRNA, a dsRNA, or a siRNA.

A cell that “stably expresses” an exogenous polypeptide is one that continues to express a polypeptide encoded by an exogenous gene introduced into the cell either after replication if the cell is dividing or for longer than a day, up to about a week, up to about two weeks, up to three weeks, up to four weeks, for several weeks, up to a month, up to two months, up to three months, for several months, up to a year or more.

A “viral vector” is defined as a recombinantly produced virus or viral particle that comprises a polynucleotide to be delivered into a host cell, either in vivo, ex vivo or in vitro. Examples of viral vectors include retroviral vectors, lentiviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827.

In aspects where gene transfer is mediated by a retroviral vector, a vector construct refers to the polynucleotide comprising the retroviral genome or part thereof, and a therapeutic gene. As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. Retroviruses carry their genetic information in the form of RNA; however, once the virus infects a cell, the RNA is reverse-transcribed into the DNA form which integrates into the genomic DNA of the infected cell. The integrated DNA form is called a provirus. As used herein, retroviral vector refers to a viral particle capable of introducing exogenous nucleic acid into a cell through a viral or viral-like entry mechanism. A “lentiviral vector” is a type of retroviral vector well-known in the art that has certain advantages in transducing nondividing cells as compared to other retroviral vectors. See, Trono D. (2002) Lentiviral vectors, New York: Spring-Verlag Berlin Heidelberg.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., International PCT Application No. WO 95/27071. Ads do not require integration into the host cell genome. Recombinant Ad derived vectors, particularly those that reduce the potential for recombination and generation of wild-type virus, have also been constructed. See, International PCT Application Nos. WO 95/00655 and WO 95/11984. Wild-type AAV has high infectivity and specificity integrating into the host cell's genome. See, Hermonat and Muzyczka (1984) Proc. Natl. Acad. Sci. USA 81:6466-6470 and Lebkowski, et al. (1988) Mol. Cell. Biol. 8:3988-3996.

As used herein, the term “AAV” is a standard abbreviation for adeno-associated virus. Adeno-associated virus is a single-stranded DNA parvovirus that grows only in cells in which certain functions are provided by a co-infecting helper virus. There are currently thirteen serotypes of AAV that have been characterized. General information and reviews of AAV can be found in, for example, Carter, Handbook of Parvoviruses 1:169-228, 1989, and Berns, Virology 1743-1764, 1999. However, it is fully expected that these same principles will be applicable to additional AAV serotypes since it is well known that the various serotypes are quite closely related, both structurally and functionally, even at the genetic level. (See, for example, Blacklowe, Parvoviruses and Human Disease 165-174, 1988, J. R. Pattison, ed.; and Rose, Comprehensive Virology 3:1-61, 1974). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “AAV expression cassette” as used herein refers to a nucleotide sequence comprising one or more polynucleotides of interest (or transgenes) that are flanked by AAV terminal repeat sequences (ITRs). Such AAV expression cassette can be replicated and packaged into infectious viral particles (e.g., AAV vectors) when present in a host cell that has been transfected with a vector encoding and expressing rep and cap gene products.

An “AAV virion” or “AAV vector” or “AAV viral particle” or “AAV vector particle” refers to a viral particle composed of at least one AAV capsid protein and an encapsidated polynucleotide AAV expression cassette. If the particle comprises a heterologous polynucleotide (i.e. a polynucleotide other than a wild-type AAV genome such as a transgene to be delivered to a mammalian cell), it is typically referred to as an “AAV vector particle” or simply an “AAV vector”. Thus, production of AAV vector particle necessarily includes production of AAV expression cassette, as such a plasmid is contained within an AAV vector particle.

Adeno-associated virus (AAV) is a replication-deficient parvovirus, the single-stranded DNA genome of which is about 4.7 kb in length including 145 nucleotide inverted terminal repeat (ITRs). There are multiple serotypes of AAV. The nucleotide sequences of the genomes of the AAV serotypes are known. For example, the nucleotide sequence of the AAV serotype 2 (AAV2) genome is presented in Srivastava et al., J Virol, 45: 555-564 (1983) as corrected by Ruffing et al., J Gen Virol, 75: 3385-3392 (1994). As other examples, the complete genome of AAV-1 is provided in GenBank Accession No. NC_002077; the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively (see also U.S. Pat. Nos. 7,282,199 and 7,790,449 relating to AAV-8); the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). Cloning of the AAVrh.74 serotype is described in Rodino-Klapac., et al. Journal of translational medicine 5, 45 (2007). Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (e.g., at AAV2 nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

Recombinant AAV genomes for use in the methods of this disclosure comprise a polynucleotide to modulate DOT1L expression and one or more AAV ITRs flanking a nucleic acid molecule. AAV DNA in the rAAV genomes may be from any AAV serotype for which a recombinant virus can be derived including, but not limited to, AAV serotypes AAVrh.74, AAVrh.10, AAVrh.20, AAV-1, AAV-2, AAV-3, AAV-4, AAV-5, AAV-6, AAV-7, AAV-8, AAV-9, AAV-10, AAV-11, AAV-12 and AAV-13. Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). As noted in the Background section above, the nucleotide sequences of the genomes of various AAV serotypes are known in the art. In some embodiments, to promote skeletal muscle specific expression, AAV1, AAV6, AAV8 or AAVrh.74 is used.

The polynucleotide that modulates DOT1L expression (e.g., a shRNA targeting DOT1L) can be under the control of a cardiomyocyte-specific promoter, e.g., the promoter of the gene encoding the contractile protein TroponinT, or myosin light chain 2 promoter (MLC-2V), or the promoter of alpha myosin heavy chain (Myh6) (see Griscelli et al. (1997) CR Acad. Sci. 329(2):103-12, or Lin Z et al. (2014) Circ Res 115:354-363, or Breckenridge R. et al (2007) Genesis 45(3):135-44, or Sohal D S et al. (2001) Circ Res 89 (1):20-5) for additional examples of tissue specific promoters.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, CA) and Promega Biotech (Madison, WI). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

“Under transcriptional control” is a term well understood in the art and indicates that transcription of a polynucleotide sequence, usually a DNA sequence, depends on its being operatively linked to an element which contributes to the initiation of, or promotes, transcription. “Operably linked” intends the polynucleotides are arranged in a manner that allows them to function in a cell.

Gene delivery vehicles also include several non-viral vectors, including DNA/liposome complexes, and targeted viral protein-DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens, e.g., a cell surface marker found on stem cells.

“RNA interference” (RNAi) refers to sequence-specific or gene specific suppression of gene expression (protein synthesis) that is mediated by short interfering RNA (siRNA).

“Short interfering RNA” (siRNA) refers to double-stranded RNA molecules (dsRNA), generally, from about 10 to about 30 nucleotides in length that are capable of mediating RNA interference (RNAi), or 11 nucleotides in length, 12 nucleotides in length, 13 nucleotides in length, 14 nucleotides in length, 15 nucleotides in length, 16 nucleotides in length, 17 nucleotides in length, 18 nucleotides in length, 19 nucleotides in length, 20 nucleotides in length, 21 nucleotides in length, 22 nucleotides in length, 23 nucleotides in length, 24 nucleotides in length, 25 nucleotides in length, 26 nucleotides in length, 27 nucleotides in length, 28 nucleotides in length, or 29 nucleotides in length. As used herein, the term siRNA includes short hairpin RNAs (shRNAs).

“Double stranded RNA” (dsRNA) refer to double stranded RNA molecules that may be of any length and may be cleaved intracellularly into smaller RNA molecules, such as siRNA. In cells that have a competent interferon response, longer dsRNA, such as those longer than about 30 base pair in length, may trigger the interferon response. In other cells that do not have a competent interferon response, dsRNA may be used to trigger specific RNAi.

The term siRNA includes short hairpin RNAs (shRNAs). shRNAs comprise a single strand of RNA that forms a stem-loop structure, where the stem consists of the complementary sense and antisense strands that comprise a double-stranded siRNA, and the loop is a linker of varying size. The stem structure of shRNAs generally is from about 10 to about 30 nucleotides in length. For example, the stem can be 10-30 nucleotides in length, or alternatively, 12-28 nucleotides in length, or alternatively, 15-25 nucleotides in length, or alternatively, 19-23 nucleotides in length, or alternatively, 21-23 nucleotides in length.

Tools to assist siRNA design are readily available to the public. For example, a computer-based siRNA design tool is available on the internet at www.dharmacon.com, Ambion-www.ambion.com/jp/techlib/misc/siRNA_finder.html; Thermo Scientific-Dharmacon-www.dharmacon.com/DesignCenter/DesignCenterPage.aspx; Bioinformatics Research Center-sysbio.kribb.re.kr:8080/AsiDesigner/menuDesigner.jsf, and Invitrogen-rnaidesigner.invitrogen.com/rnaiexpress/.

shRNA that targets and downregulates DOT1L can be prepared using methods known in the art (see, e.g., Cattaneo et al. (2016) Cell Death and Differentiation, 23:555-564 or are commercially available, see e.g., Vector Biolabs, Cat. No.: shAAV-207261, https://www.vectorbiolabs.com/product/shaav-207261-human-dot1l-shrna-silencing-aav/. Examples of target sequences that can be used for shRNA-mediated silencing of human DOT1L include CGCCAACACGAGTGTTATATT (within the coding sequence, clone ID TRCN0000020209) or CACGTTGAACAAGTGCATTTA (within the 3′ untranslated region, clone ID TRCN0000236343).

CRISPR systems can also be used to downregulate or abrogate DOT1L expression by inhibiting transcription (also designated CRISPRi) using dCas9 (a mutant version of the Cas9 enzyme that lacks endonuclease activity) fused to a repressor domain (for example the Kruppel Associated Box—KRAB—domain) that is guided to the DOT1L gene by a single guide RNA designed to specifically target the promoter or exons of this gene, thereby repressing its transcription. Crispr regulation of translation, on the other hand, employs a catalytically dead dCas13 that targets RNA molecules (unlike Cas9 that targets DNA). In this case, a guide RNA targeting the translation start site of the DOT1L mRNA is used to ensure that dCas13 selectively represses production of DOT1L protein, without affecting translation of mRNAs encoding other proteins.

The Kruppel associated box (KRAB) domain is a category of transcriptional repression domains present in approximately 400 human zinc finger protein-based transcription factors (KRAB zinc finger proteins). The KRAB domain typically consists of about 75 amino acid residues, while the minimal repression module is approximately 45 amino acid residues. It is predicted to function through protein-protein interactions via two amphipathic helices. The most prominent interacting protein is called TRIM28 initially visualized as SMP1, cloned as KAP1- and TIF1-beta Substitutions for the conserved residues abolish repression.

As used herein, the term “administer” or “administration” or “administering” intends to mean delivery of a substance to a subject such as an animal or human. Administration can be effected in one dose, continuously or intermittently throughout the course of treatment. Methods of determining the most effective means and dosage of administration are known to those of skill in the art and will vary with the composition used for therapy, the purpose of the therapy, as well as the age, health or gender of the subject being treated. Single or multiple administrations can be carried out with the dose level and pattern being selected by the treating physician or in the case of pets and animals, treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated and the target cell or tissue. Non-limiting examples of route of administration include intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmuccosal, and inhalation.

An agent of the present disclosure can be administered for therapy by any suitable route of administration. It will also be appreciated that the optimal route will vary with the condition and age of the recipient, and the disease being treated. These strategies can be administered acutely (in a scenario of myocardial infarction) or chronically in any setting of heart failure. They can be administered systemically or locally. Non-limiting examples of locally delivery includes intracardiac injection or delivery during reperfusion of a coronary artery.

Cardiac injury intends any injury to the heart muscle or cardiac cells. Non-limiting examples include heart failure, blunt cardiac injury, coronary artery disease, ischemic heart disease, or myocardial infarction. The injury can be acute or chronic.

Heart failure, also known as congestive heart failure, is a condition that develops when the heart does not pump enough blood to meet the body needs. “Therapeutically effective amount” of a drug or an agent refers to an amount of the drug or the agent that is an amount sufficient to obtain a pharmacological response such as passive immunity; or alternatively, is an amount of the drug or agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose, and may occur only after administration of a series of doses. Thus, a therapeutically effective amount may be administered in one or more administrations.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound.

### Modes For Carrying Out the Disclosure

Provided herein is a method to modulate cardiogenesis in a mammalian cardiac cell or mammalian cardiac cell progenitor, comprising contacting the cell with a DOT1L gene modulator. In one aspect, the DOT1L gene modulator is an agent that upregulates DOT1L gene expression or function in the cell. Non-limiting examples of upregulators of DOT1L function include introducing into the cell a polynucleotide encoding DOT1L such a polynucleotide encoding mammalian DOT1L. The polynucleotide can be a DNA or RNA sequence and can be introduced by a vector, such as a viral vector using methods known in the art. A non-limiting example of a vector is an expression vector such as an AAV vector. The polynucleotide can be operably linked to a muscle-specific promoter.

In one aspect, the mammalian cell is a post-natal cardiac cell. In another aspect, the cell is an isolated cell. Non-limiting examples of mammalian cells include a cell selected from a canine cell, an equine cell, a feline cell, a murine cell or a human cell. In another aspect, the DOT1L gene modulator is an agent that downregulates or abolishes DOT1L gene expression, or inhibits the DOT1L function in the cell. An example of such is a chemical inhibitor of DOT1L activity, non-limiting examples of such include EPZ004777, SGC0946 and EPZ-5676. Another example is shRNA or siRNA that targets DOT1L that can be delivered with a gene expression vector or lipid nanoparticle. The shRNA can be under the control of a cardiac-specific promoter.

In one aspect, the gene modulator comprises a system that reduces or abrogates endogenous DOT1L gene expression, such as a gene editing system such as CRISPR or other gene editing system described herein. Alternatively, the agent can be one known in the art, see, e.g., Dickins et al. (2020) Blood 136(17): 1900-1901; Perner et al. (2020) Blood, 136(17):1983-1988; Yang, et al. (2019 Clinical Epigenetics Vo. 11 (https://clinicalepigeneticsjoumal.biomedcentral.com/articles/10.1186/s13148-019-0778-y); SYC-522 (Sigma Aldrich); and SGC-0946 (MeKoo Biosciences, Inc.).

Non-limiting examples of mammalian cells include a cell selected from a canine cell, an equine cell, a feline cell, a murine cell or a human cell.

Also provided is a method of promoting de novo cell cycle of a post-mitotic mammalian cardiac cell, comprising contacting the cell with an agent that inhibits expression or function of an endogenous DOT1L gene in the cell, thereby promoting cardiac regeneration or de novo cell cycle of the post-mitotic mammalian cardiac cell. In one aspect, the mammalian cell is a post-natal mammalian cardiac cell. In another aspect, the cell is an isolated cell. In a further aspect, the mammalian cell is a human cell. In another aspect, the cardiac cell is a cell post-injury.

In a further aspect, the agent reduces or abrogates endogenous DOT1L gene expression in the cell is an agent that downregulates or abolishes DOT1L gene expression, or inhibits the DOT1L function in the cell. An example of such is a chemical inhibitor of DOT1L activity, non-limiting examples of such include EPZ004777, SGC0946 and EPZ-5676. Another example is shRNA or siRNA that targets DOT1L that can be delivered with a gene expression vector or lipid nanoparticle. The shRNA can be under the control of a cardiac-specific promoter, siRNA that targets DOT1L. In another aspect, the gene modulator comprises a system that reduces or abrogates endogenous DOT1L gene expression, such as a gene editing system such as CRISPR or other gene editing system as shown herein. Alternatively, the agent can be one known in the art, see, e.g., Dickins et al. (2020) Blood 136(17): 1900-1901; Perner et al. (2020) Blood, 136(17):1983-1988; Yang, et al. (2019 Clinical Epigenetics Vo. 11 (https://clinicalepigeneticsjoumal.biomedcentral.com/articles/10.1186/sI3148-019-0778-y); SYC-522 (Sigma Aldrich); and SGC-0946 (MeKoo Biosciences, Inc.).

Non-limiting examples of mammalian cells include a cell selected from a canine cell, an equine cell, a feline cell, a murine cell or a human cell.

The contacting can be performed is in vitro or in vivo. When in vitro, the agent is contacted with the cell, typically in a cell culture system. The agent can be combined with a carrier, such as a pharmaceutically acceptable carrier, for ease of use. The in vitro systems are useful to assay for new combinations or test for new agents that have the same or similar effect. As is apparent to the skilled artisan, an effective amount of the agent must be contacted with the cell, e.g., the mammalian cell. When performed in vivo, the method is a powerful therapy. When the mammal is a non-human mammal, the method can be used as an animal model to test new therapies and combinations. It also can be used to treat pets and farm animals and thus, also provided is a therapeutic method. When performed in a human, the method is a powerful therapy that can be used alone or in combination with other known therapies. When administered in vivo, an effective amount of the agent is administered which is determined by the treating professional and will vary with the health and age of the mammal, as well as the condition to be treated. The agent can be administered by any suitable route, locally or systemically (intravenous for example). In another aspect, the agent is administered locally via intracardiac injection or delivery during reperfusion of the obstructed coronary.

In addition, any agent as described herein can be combined with a carrier such as a pharmaceutically acceptable carrier. Also provided is a method of treating cardiac injury or heart failure in a subject in need thereof comprising administering locally or systemically an effective amount of an agent that reduced or abrogates DOT1L function in a cardiac cell or tissue, thereby treating cardiac injury or heart failure. Non-limiting examples include heart failure, blunt cardiac injury, coronary artery disease, ischemic heart disease, or myocardial infarction. The injury can be acute or chronic.

An agent reduces or abrogates endogenous DOT1L gene expression in the cell is an agent that downregulates or abolishes DOT1L gene expression, or inhibits the DOT1L function in the cell. An example of such is a chemical inhibitor of DOT1L activity, non-limiting examples of such include EPZ004777, SGC0946 and EPZ-5676. Another example is shRNA or siRNA that targets DOT1L that can be delivered with a gene expression vector or lipid nanoparticle. The shRNA can be under the control of a cardiac-specific promoter, siRNA that targets DOT1L. In another aspect, the gene modulator comprises a system that reduces or abrogates endogenous DOT1L gene expression, such as a gene editing system such as CRISPR or other gene editing system as shown herein. Alternatively, the agent can be one known in the art, see, e.g., Dickins et al. (2020) Blood 136(17): 1900-1901; Perner et al. (2020) Blood, 136(17):1983-1988; Yang, et al. (2019 Clinical Epigenetics Vo. 11 (https://clinicalepigeneticsjoumal.biomedcentral.com/articles/10.1186/s13148-019-0778-y); SYC-522 (Sigma Aldrich); and SGC-0946 (MeKoo Biosciences, Inc.).

The agent can be combined with a carrier, such as a pharmaceutically acceptable carrier, for ease of use. An effective amount of the agent is administered, which is determined by the treating professional and will vary with the health and age of the mammal, as well as the condition to be treated. The agent can be administered by any suitable route, locally or systemically (intravenous for example). In another aspect, the agent is administered locally via intracardiac injection or delivery during reperfusion of the obstructed coronary.

Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human.

The following examples are provided to illustrate the concepts of this disclosure.

### Experimental

### Experiment No. 1

Epigenetic enzymes play critical roles in organogenesis by defining chromatin structures required for cell type-specific transcriptional networks¹. Yet, mechanisms by which genome-wide histone modifications result in activation or repression of specific genes remain little understood. DOT1L is the only epigenetic enzyme catalyzing methylation of lysine 79 of histone 3 (H3K79me)^{2,3}. Originally identified in yeast due to its role in maintenance of telomeric regions², DOT1L has been extensively studied in MLL-rearranged leukemia, where it is considered an emerging therapeutic target⁴. Initial genome-wide studies suggested that, rather than being associated with specific gene expression programs, H3K79me2/3 is ubiquitously found in all transcribed loci, leading to a model of H3K79me as a generic mark of active genes⁵.

Recent studies have revealed that, in addition to gene body H3K79me, DOT1L can regulate expression of its targets via methylation of regulatory regions⁶. Applicant previously showed that, in vitro, DOT1L is required for proper differentiation of embryonic stem cells into cardiomyocytes (CMs)⁷, however it is currently unclear whether this enzyme is necessary for normal cardiogenesis in vivo. In mice, global ablation of Dot1l results in embryonic lethality between E9.5 and E10.5 with mutants displaying yolk sac angiogenic defects, growth impairment and cardiac dilation⁸. Selective Dot1l ablation in CMs using αMHC-Cre results in an adult lethal phenotype with perturbations in dystrophin expression⁹. However, owing to the nature of this knockout (using a Cre that is not active during early embryogenesis^{10,11}), it is not known whether DOT1L plays any role in cardiogenesis. Furthermore, Applicant's molecular understanding of the functioning of this enzyme in cardiogenesis in vivo is limited by the absence of RNA-seq or ChIP-seq datasets that would allow for unbiased identification of direct targets of DOT1L in embryonic or neonatal CMs.

The mammalian heart develops from two major populations of cardiogenic progenitors—the first and second heart fields. Cells from the first heart field give rise to the left ventricle, whereas cells from the second heart field give rise to the right ventricle and most of the atria¹². A set of asymmetrically expressed transcription factors are essential for the establishment of chamber-specific transcriptional programs that regulate cardiac patterning and chamber maturation¹³⁻¹⁶. Amongst these, Irx4 (expressed only in ventricular CMs but absent from the atria) is essential to establish a ventricular identity in lieu of an atrial phenotype¹⁷. Hand1 (expressed in the left ventricle throughout development and in the cardiac conduction system post-birth¹⁸) and Hand2 (restricted to the right ventricle in early patterning and subsequently expressed in endocardium and myocardium of both ventricles) play a major role in defining systemic and pulmonary ventricular identity, respectively¹⁹⁻²¹ Epigenetic mechanisms contributing to the tightly regulated expression of chamber-specific genes remain mostly unexplored, however, it is known that the H3K4 methyltransferase SMYD1/BOP1 is a critical regulator of Hand2 expression²². To date, it is not known whether any particular epigenetic enzyme plays a similar role in left ventricle-specific regulation of Hand1 expression.

Regulated CM proliferation is a major component of normal cardiogenesis. The rate of CM mitosis gradually decreases from midgestational stages until birth, reaching a state of complete mitotic withdrawal in the first week after birth, when the majority of CMs become binucleated²³. Therefore, a major goal in cardiac regeneration is the identification of strategies to promote proliferation of adults CMs.

To test whether DOT1L plays a role in cardiogenesis, Applicant ablated a floxed Dot1l allele using a Cre allele that is active in CMs from early developmental timepoints and observed a perinatal lethal phenotype with abnormal cardiac morphology. This phenotype resulted from perturbations in highly specific gene expression programs that orchestrate normal cardiogenesis at two distinct timepoints: embryonic heart development and neonatal CM cell cycle withdrawal. In embryogenesis, DOT1L emerged as a major regulator of the expression of several transcription factors involved in chamber-specific gene expression profiles, with left ventricle-specific genes being particularly sensitive to the absence of this enzyme. In the neonatal period, DOT1L promoted expression of genes involved in CM maturation and cell cycle exit, with Dot1L knockouts exhibiting sustained CM proliferation, a feature of interest for cardiac regeneration. Integration of CM ChIP-seq and Hi-C data revealed that DOT1L regulated target genes via two mechanisms: methylation of H3K79 in gene bodies and methylation of H3K79 in regulatory elements (K79-REs). Applicant's analyses identified two types of K79-REs: activating elements that synergized with gene body H3K79me2 to promote expression of targets activated by DOT1L, and K79-REs that contributed to silencing of genes normally not expressed in CMs. In contrast to H3K79me2 being a general activator of gene transcription as previously thought, Applicant's results reveal H3K79me2 as an epigenetic mark that regulates highly specific gene programs by both activating and repressing target genes.

Abnormal Cardiogenesis in DOT1L cKOs

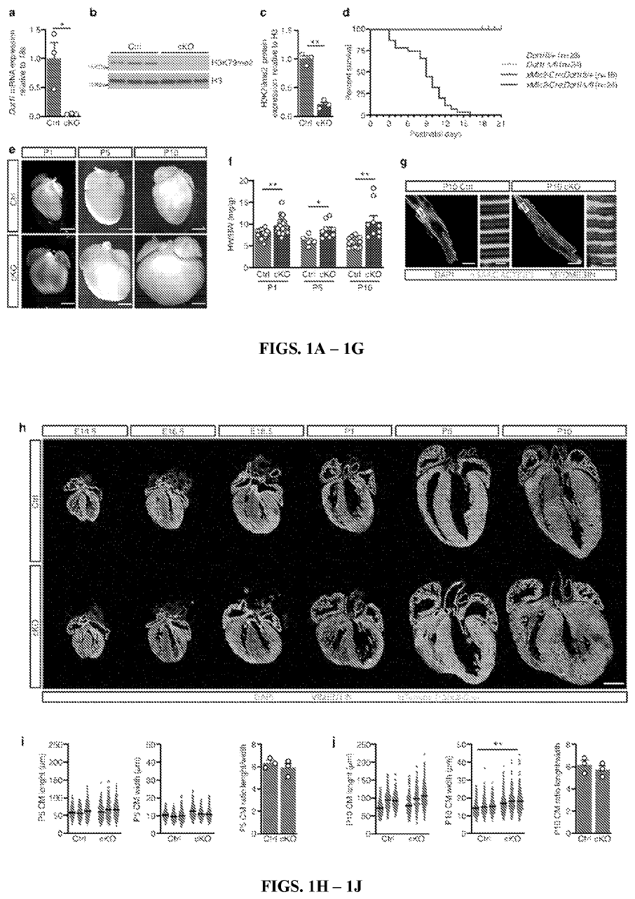

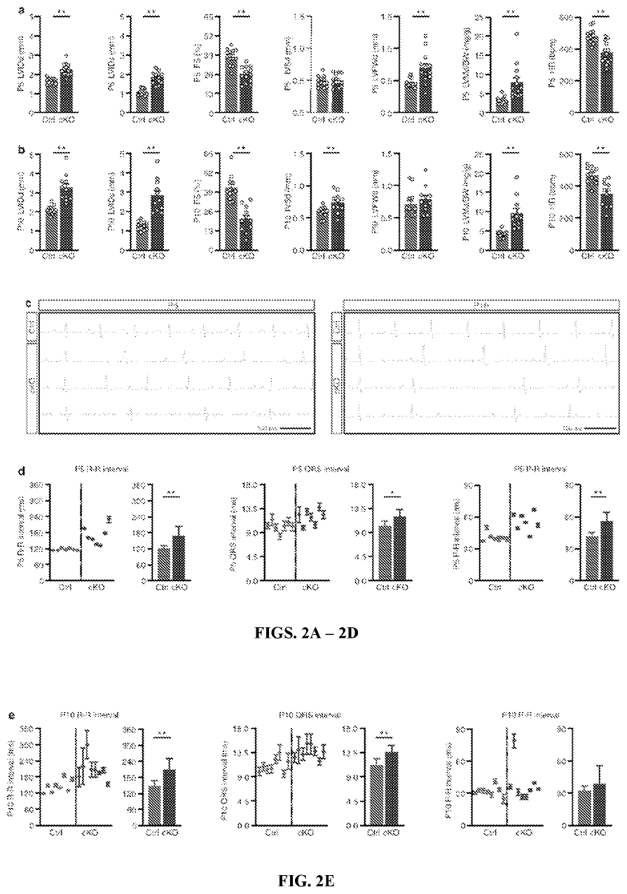

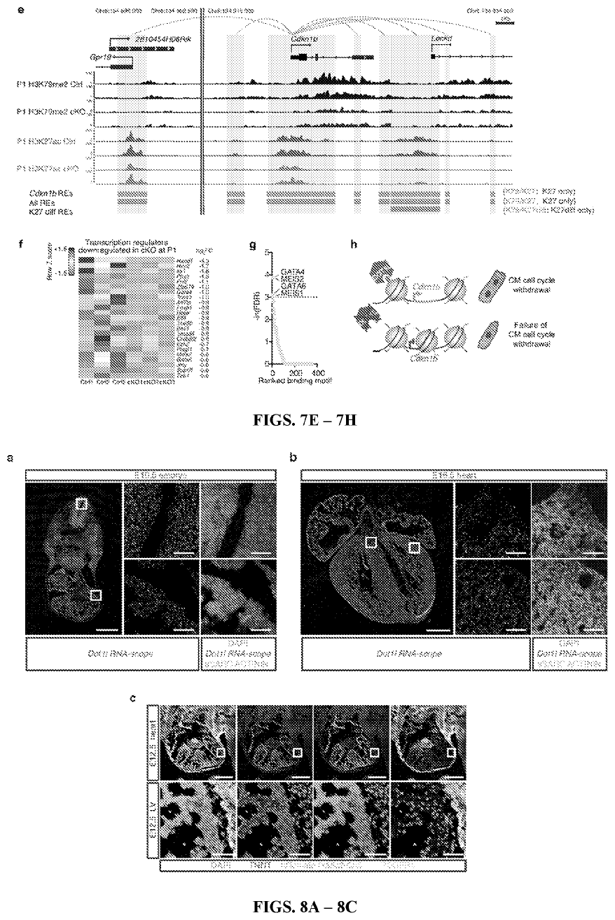

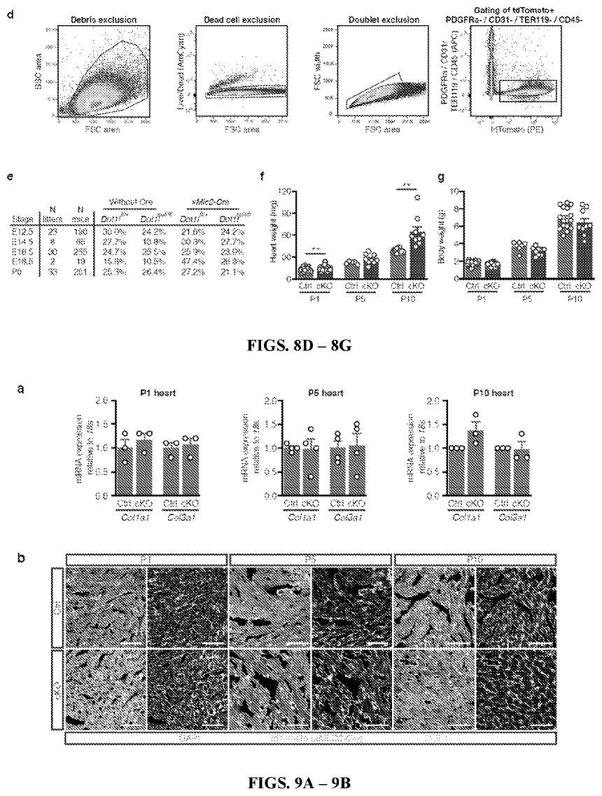

Following Applicant's observation that DOT1L is required for the differentiation of embryonic stem cells into CMs⁷, Applicant assessed if this enzyme is required for normal cardiogenesis in vivo. Fluorescent in situ hybridization studies showed that the Dot1l gene is robustly expressed in CMs at least from E10.5 onwards (FIG. 8A). This expression was observed throughout the heart, without being specific to any chamber. Dot1l expression was also observed in non-cardiomyocyte cells of the heart, as well as most tissues outside the heart, showing this enzyme is not cardiac-specific. However, at E16.5, Dot1l expression levels were higher in CMs than in the developing valves (FIG. 8B). Dot1l has been previously ablated in the heart using an αMHC-Cre line, leading to an adult lethal phenotype with reduced dystrophin expression⁹. Applicant demonstrated that αMHC-Cre is not optimal for myocardial-restricted knockouts due to its expression in non-myocyte lineages inside and outside the heart, and because it does not flox-out in all CMs at an early time point^{10,11}. To avoid these limitations and study CM-specific roles of DOT1L from early embryonic timepoints, Applicant ablated a floxed Dot1l allele (loxP sites flanking exon 2) using the xMlc2-Cre allele²⁴ that drives highly specific and efficient recombination in CMs (FIG. 8C) from the cardiac crescent stage²⁴. In Applicant's analyses, xMlc2-Cre+; Dotll-WTflox mice (herein designated as Dot1L Ctrl or controls) were compared with xMlc2-Cre+; Dotll-z/flox littermate mice (herein designated as Dot1L cKO or mutants). Inclusion of a copy of the Rosa26-tdTomato reporter allele²⁵ in all crosses allowed highly specific labeling of CMs by the red fluorescent protein tdTomato for downstream flow cytometry and confocal microscopy applications (FIG. 8D). Real time qPCR analyses revealed highly efficient ablation of the floxed allele in FACS-sorted E12.5 CMs (FIG. 1A).

DOT1L is the only enzyme catalyzing H3K79 methylation³. Consequently, CM ablation of Dot1l resulted in a strong reduction in H3K79me2 levels in E14.5 hearts (FIG. 1B and FIG. 1C). CM-specific ablation of Dot1l did not result in an embryonic lethal phenotype, as animals with the mutant genotype were observed at expected Mendelian ratios in all stages analyzed (FIG. 8E). Despite being born at expected numbers, Dot1L cKO mice started dying shortly after birth, with 50% mortality by postnatal day 9 (P9), and the longest-lived mutant surviving until P16 (FIG. 1D). Macroscopic examination of organs at neonatal stages revealed that mutants had enlarged hearts with a rounded shape relative to controls (FIG. 1E). This phenotype was exacerbated as animals aged and peaked at P10 in surviving animals (FIG. 1E). This evident cardiac enlargement translated into increased heart weight (FIG. 8F) and increased heart weight/body weight ratios (FIG. 1F) without changes in body weight (FIG. 8G). Co-immunostaining for αSarcomeric Actinin and Myomesin revealed absence of myofiber orientation defects in CMs from Dot1L cKO hearts (FIG. 1G).

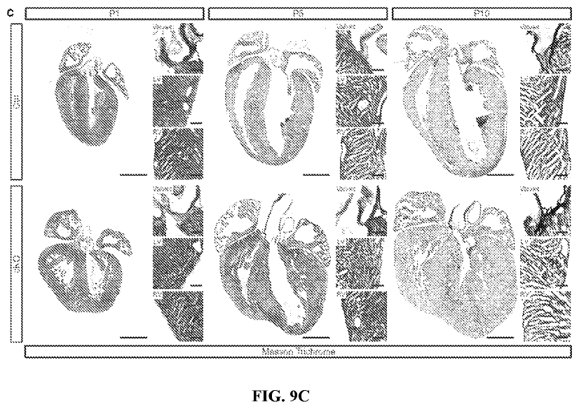

To determine the timing of onset of the cardiac phenotype and assess for additional morphogenic malformations, a histological time course analysis was conducted spanning from midgestation to postnatal timepoints (FIG. 1H). cKO hearts were completely indistinguishable from control littermates until E14.5 and started exhibiting the enlarged, rounded phenotype between E16.5 and E18.5. Postnatally, mutant hearts displayed ventricular walls with increased thickness and areas of moderate persistent trabeculation (FIG. 1H). No additional major morphogenic abnormalities (septal or outflow defects) were observed. Immunostaining for Vimentin revealed absence of valve malformations and absence of major foci of fibrosis in cKOs (FIG. 1H). Absence of fibrotic remodeling was also confirmed by qPCR for fibrotic markers Col1a1 and Col3a1 (FIG. 9A) performed on whole heart tissue, immunostaining for Collagen1 (FIG. 9A), and Masson trichrome staining (FIG. 9C). To assess if the cardiac enlargement observed in cKOs is a consequence of CM hypertrophy, length and width measurements of CMs isolated from Dot1L cKO and control hearts were conducted. These analyses revealed that, at P5, CMs from both genotypes had comparable sizes (FIG. 1I). At P10, CM length was similar in both groups, but CM width was moderately increased in cKOs, without causing a significant change in the CM length-to-width ratio (FIG. 1J). These results ruled out CM hypertrophy as a cause of the observed cardiac enlargement.

Echocardiographic assessment of heart function revealed that, compared with littermate controls, Dot1L cKO mice had reduced fractional shortening both at P5 and P10, increased left ventricular posterior wall thickness and increased left ventricular chamber diameter (FIG. 2A and FIG. 2B). Electrocardiographic analyses revealed that Dot1L cKO mutants had reduced heart rate both at P5 and P10, which translated into prolonged and irregular R-R intervals, with occasional beat drops (FIG. 2C, FIG. 2D, FIG. 2E). At P5, Dot1L cKOs also had prolonged QRS and P-R intervals, whereas at P10 the P-R intervals of mutants were comparable to those of controls, suggesting that the mutants with more severe conduction defects might die between P5 and P10. Despite the strong cardiac phenotype, Dot1L cKOs presented no obvious signal of distress prior to death. Therefore, it is possible that defects in cardiac conduction might account for the sudden lethal phenotype.

Altogether, these findings identify a critical role for DOT1L in normal cardiogenesis and suggest that the strong macroscopic and functional phenotypes observed in Dot1L cKOs are not a consequence of fibrotic remodeling or major alterations in CM structure or size.

### DOT1L is Essential for Chamber-Specific Gene Expression

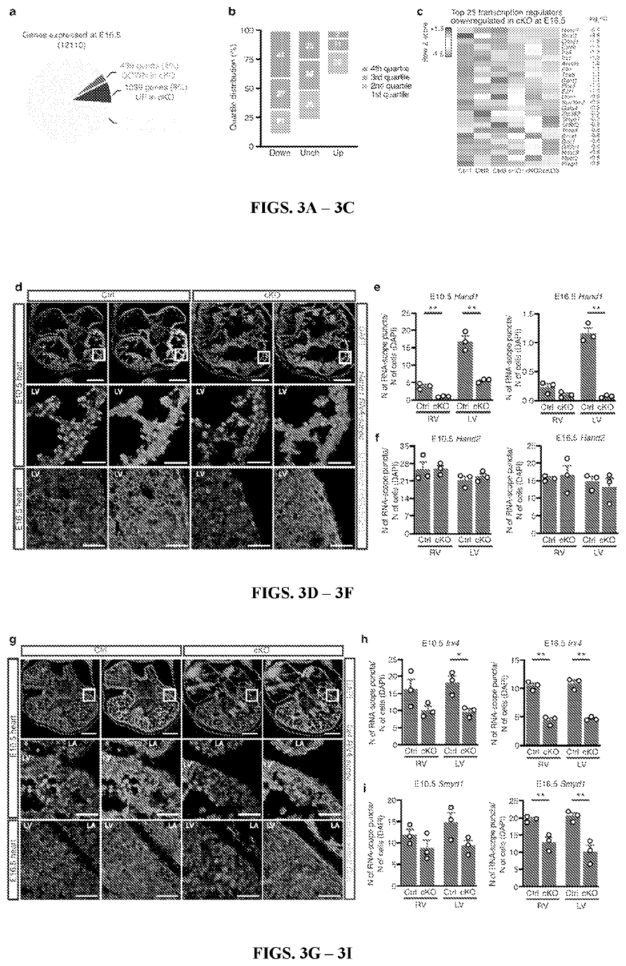

To decipher gene expression networks misregulated in the absence of DOT1L, RNA-seq was used to assess the transcriptomes of FACS-sorted Ctrl and cKO CMs just prior to the onset of clear phenotypic changes (E16.5). An average of 100,000 CMs were sorted per E16.5 heart (including CMs from all four cardiac chambers), yielding enough RNA to prepare biological replicates from individual hearts (3 replicates per genotype group). Bioinformatics analyses revealed that, from a total of 12,110 expressed genes (RPKM≥1 in either Ctrl or cKO), 1,478 were misregulated in cKO versus Ctrl CMs (log₂FC≤−0.5 or ≥0.5, false discovery rate≤0.05). From these, 439 genes were downregulated and 1,039 upregulated in cKOs (FIG. 3A and FIG. 8A). Transcripts upregulated in cKOs corresponded to genes that are normally expressed at low levels in control CMs (62% of genes in the lower expression quartile), whereas downregulated genes corresponded to genes normally expressed at a medium/high level (FIG. 3B). Notably, the gene displaying the highest downregulation (−5.4 log₂FC) in mutant CMs was the transcription factor Hand1, a central element in the establishment and maintenance of left ventricular identity (FIG. 3C and FIG. 8A). In addition, amongst genes significantly downregulated in mutants there were several other genes asymmetrically expressed across the cardiac chambers: the transcription factors Irx4 (ventricular specific¹⁷), Tbx5 (left ventricle and atrial-specific²⁶), and the left ventricular-specific genes Gja5 (encoding Connexin40) and Cited1 (FIG. 3C and FIG. 8A). These results revealed that transcriptional networks operating in left ventricular CMs (those derived from the first heart field) are particularly sensitive to the absence of DOT1L. In addition to chamber-specific genes, DOT1L also regulated critical cardiogenic factors that are expressed throughout the heart. Examples of these are the transcription factors Gata4 andMef2c, as well as the epigenetic enzyme Smyd1. The transcription factor Nkx2-5 also exhibited a trend toward downregulation (FIG. 8A). These transcriptomic differences revealed that DOT1L activity in embryonic CMs is essential for establishment of gene expression networks that coordinate normal cardiogenesis. This requirement, however, was not generalized to all cardiac patterning genes, as transcript levels for Hand2, Tbx20 and Nppa (ANF) were not altered in mutants (FIG. 8A).

To validate RNA-seq results, Applicant performed fluorescent RNA in situ hybridization (RNA-scope) in histological sections of control and cKO hearts. Strikingly, at E16.5, Hand1 transcripts were completely undetectable in the left ventricle of mutants (FIG. 3D, bottom panel, quantification in FIG. 3E, right). Downregulation of Hand1 was already evident at E10.5 when levels of Hand1 expression in control hearts were higher than at E16.5 (FIG. 3D, top and middle panels, quantification in FIG. 3E, left), indicating that transcriptional consequences of DOT1L ablation preceded the first phenotypic manifestations by several days. Transcript levels for the ventricular-specific transcription factor Irx4 and for the CM-specific epigenetic modifier Smyd1 were significantly reduced in cKO hearts both at E10.5 and E16.5 (FIG. 3G and FIG. 10, quantification in FIG. 3H and FIG. 31), confirming RNA-seq results. RNA-scope analyses also showed that Dot1L cKO CMs expressed normal levels of Hand2, both at E10.5 and E16.5, further validating that DOT1L regulates specific transcriptional programs, rather than being a generic activator of transcription (FIG. 1I, quantification in FIG. 3F).

Applicant's data revealed a previously unrecognized role for DOT1L in regulating the expression of genes necessary for the establishment and maintenance of chamber identity, in particular those involved in left ventricular identity. While a number of genes were downregulated in cKOs, Hand1 was particularly sensitive to absence of DOT1L, with Hand1 transcripts being completely absent from midgestational DOT1L-deficient CMs (both in RNA-seq and RNA-scope). Consistent with these transcriptomic observations, cardiac-specific ablation of Hand1 phenocopies Dot1L cKO, including an enlarged and round-shaped heart and peri-natal lethality²¹.

### Transcriptional Control Via Gene Body H3K79Me2

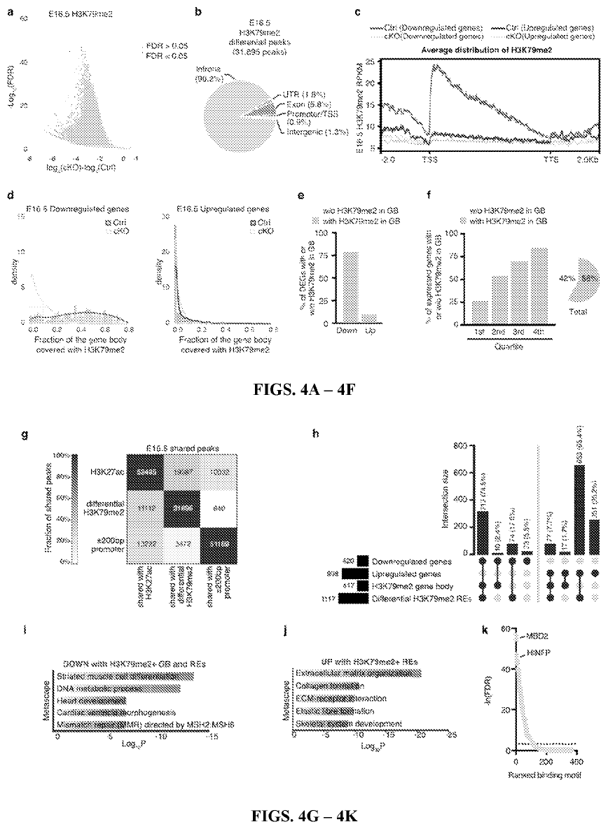

Several core cardiac transcription factors are differentially expressed in Dot1L cKO CMs. Thus, genes modulated in our transcriptomic analyses likely include a combination of targets directly regulated by DOT1L as well as those indirectly regulated owing to secondary effects. To identify genes directly regulated by DOT1L, we performed ChIP-seq assays for H3K79me2 in E16.5 control and cKO FACS-sorted CMs. These analyses revealed a total of 31,895 H3K79me2 peaks significantly enriched in control versus Dot1L cKO CMs (FIG. 9A). The fact that the vast majority of H3K79me2 peaks in Ctrls were lost in cKOs is consistent with DOT1L being the sole histone K79 methyltransferase and validated the purity of Applicant's sorted CMs (FIG. 4A and FIG. 9A). Genome-wide differential peak distribution analysis revealed that H3K79me2 is mostly an intragenic modification (introns, exons, UTR and promoter/TSS) with only 1% of differential peaks being located in intergenic regions (FIG. 4B).

Genes downregulated in cKOs exhibited, in control CMs, maximal levels of H3K79me2 immediately after the transcription start site (TSS) that progressively decreased towards the transcription termination site (TTS) (FIG. 4C). Gene body H3K79me2 (coverage≥50 reads and fraction of gene body≥0.20 in control CMs) was present in 341 downregulated genes (77% of all downregulated genes) and 98 upregulated genes (9% of all upregulated genes) (FIG. 4D, FIG. 4E and FIG. 8A). This observation suggests that a significant part of gene downregulation observed in cKOs can be directly attributed to H3K79me2 in the gene body, whereas gene upregulation does not seem to be directly associated with gene body H3K79me2. This is consistent with the view of gene body H3K79me2 as an activation mark⁵, however, it should be noted that this modification is not an absolute requirement for transcription, as 42% of all genes expressed in control CMs did not have significant levels of gene body H3K79me2 (FIG. 4F). Within genes showing gene body H3K79me2, there were two clear categories: genes dependent on DOT1L (modulated in cKOs) and genes not significantly affected by the loss of DOT1L, suggesting a scenario in which other epigenetic modifications can compensate for the loss of gene body H3K79me2 in a subset of genes. Notably, the majority of genes whose transcription depended on gene body H3K79me2 belonged to the highest quartiles of expression, suggesting this modification might play a role in supporting high transcriptional levels (FIG. 4F).

### Transcriptional Control Via H3K79Me2 in Regulatory Elements

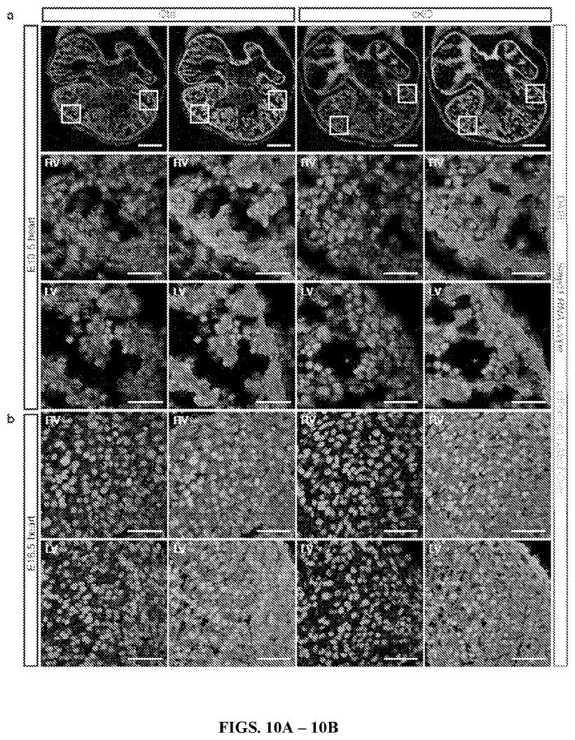

Recent studies in leukemia showed that there is a distinct subset of enhancers that depend on H3K79me2⁶. Applicant conducted analyses to investigate whether CMs have H3K79me2-dependent regulatory elements (REs) that, together with gene body H3K79me2, contribute to gene expression differences observed in cKOs. To identify regulatory regions, Applicant generated CM-specific H3K27ac ChIP-seq datasets. At E16.5, control CMs had 52,495 H3K27ac peaks, mainly occupying introns, promoters/TSS or intergenic regions (FIG. 12A). From these, 37% overlapped with H3K79me2 peaks (FIG. 4G and FIG. 12B). To predict interactions between candidate regulatory elements and target genes, Applicant applied an adapted implementation of the Activity-by-Contact (ABC) model^{27,28}. By integrating information on chromatin state (H3K27ac ChIP-seq) and genomic conformation (Hi-C), this method is more accurate at predicting actual interactions between REs and their target genes than methods simply assigning H3K27ac peaks to the closest neighboring gene²⁷. Because the promoter of a gene can serve as a RE for a distinct cis gene^{29,30}, Applicant's analyses also considered interactions between promoters and distal genes. Using the ABC algorithm, our CM-specific H3K27ac ChIP-seq dataset was combined with a publicly available CM Hi-C dataset³¹ (GSM2544836). To ensure focus on meaningful candidate RE-target gene interactions, only the most relevant interactions identified by the ABC method were considered (adapted ABC-score≥0.02): 373,610 interactions, corresponding to 47,547 H3K27ac peaks (FIG. 10A and FIG. 12C). From these, 36% (16,963 peaks) overlapped with a differential H3K79me2 peak (FIG. 10A and FIG. 12C). This large number of CM REs with H3K79me2 signal (from here on designated as K79-REs) could provide an alternative mechanism for gene expression regulation by DOT1L. Of note, about 34% of all K79-REs observed at E16.5 overlapped a promoter (FIG. 10A and FIG. 12C).

To assess the relative contribution of H3K79me2 in gene body versus H3K79me2 in REs to the regulation of target genes, Applicant quantified the effect of these variables in the cumulative distribution of log₂FC values. These analyses revealed that genes with higher fraction of H3K79me2 in the gene body were more downregulated in cKO than genes with lower (less than 33%) fraction of H3K79me2 in the gene body (FIG. 12D and FIG. 11A). Genes with 6 or more K79-REs displayed stronger modulation than those associated with lower numbers of REs (FIG. 12E and FIG. 11A). Focusing exclusively on loci with gene body H3K79me2, those that interacted with K79-REs were more downregulated in mutant CMs than those without these elements, indicating that gene body and regulatory element H3K79me2 synergized to potentiate expression of target genes (FIG. 12F and FIG. 11A).

The vast majority (74.5%) of the genes downregulated in DOT1L-deficient CMs were associated with both gene body H3K79me2 and at least one K79-RE (FIG. 4H). Interestingly, 17.6% of all downregulated genes were associated with K79-REs in the absence of gene body H3K79me2. Overall, only 5.5% of downregulated genes were not associated with H3K79me2 in gene body or K79-REs, indicating most gene downregulation was a direct consequence of Dot1L cKO. On the other side of the scale, upregulated genes had reduced association with gene body H3K79me2 (as mentioned above), but, surprisingly, 65.4% of upregulated genes were associated with K79-REs in the absence of gene body H3K79me2. This observation suggests a role for DOT1L in gene silencing via H3K79me2-dependent regulatory elements. Functional annotation revealed that genes downregulated in cKO with H3K79me2 in gene bodies and REs were involved in differentiation of striated muscle and cardiac morphogenesis (FIG. 4I and FIG. 12A). On the other hand, genes upregulated in cKOs and associated with inhibitory K79-REs were related to non-myocyte functions: extracellular matrix organization and skeletal system development (FIG. 4J and FIG. 12B).

To search for cues as to how DOT1L achieves gene silencing via methylation of REs, Applicant screened for enrichment in binding sites for known transcriptional regulators in the K79-REs of genes upregulated without gene body H3K79me2 but with K79-REs versus genes downregulated with gene body H3K79me2 and K79-REs. This analysis revealed that K79-REs associated with genes upregulated in cKOs (silencing K79-REs) were enriched in binding sites for MBD2, a member of the NuRD complex known to play an important role in transcriptional silencing³² (FIG. 4K). Enrichment in NuRD complex binding sites in the silencing K79-REs could provide an explanation for how DOT1L promotes silencing of target genes. However, it is also possible that upregulated genes reflect secondary effects (for example downregulation of a transcription regulator with repressor properties) rather than being a direct consequence of DOT1L inactivation.

### H3K27ac Relationship to H3K79me2

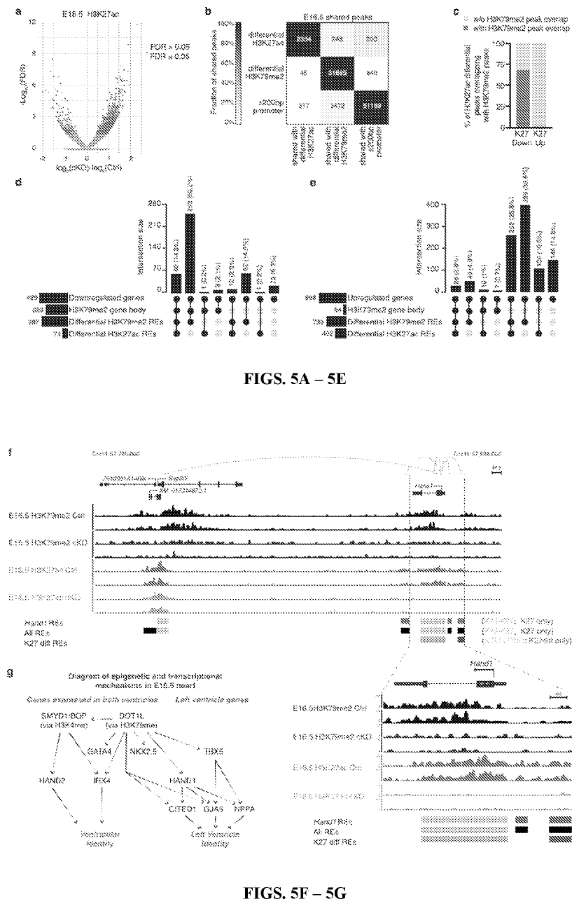

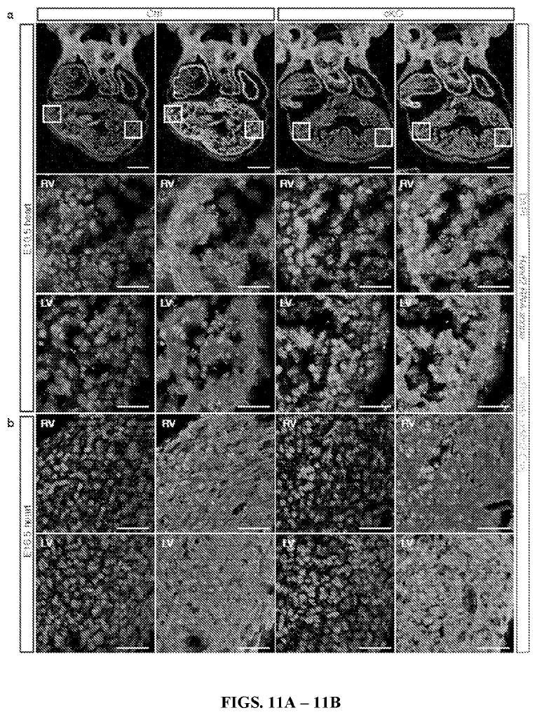

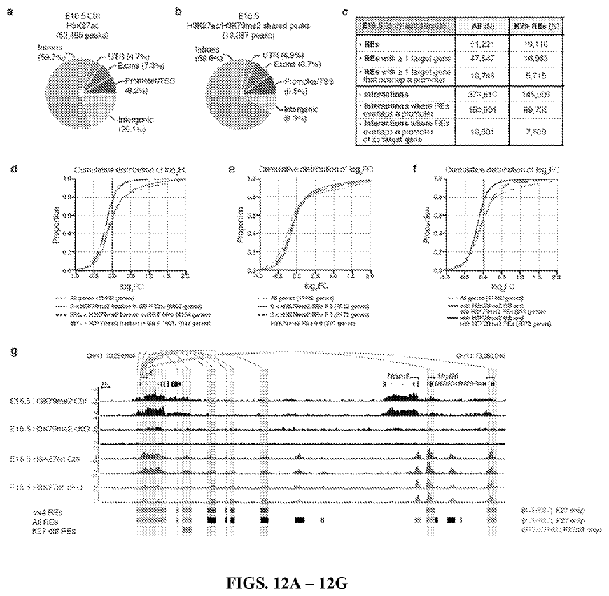

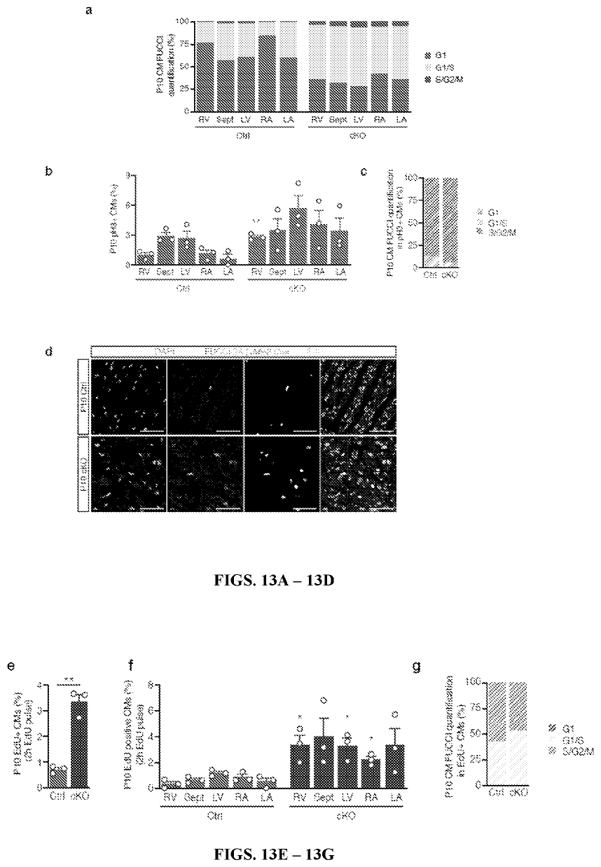

To assess whether H3K27ac in K79-REs is altered upon DOT1L ablation, Applicant also performed H3K27ac ChIP-seq in E16.5 CMs sorted from cKO hearts. Comparison of ChIP-seq profiles from control and mutant CMs identified 2,354 differential H3K27ac peaks (FIG. 5A, FIG. 5B and FIG. 13A). From these, 2,017 were upregulated and 337 downregulated in cKOs (FIG. 5A and FIG. 13A). Most (69%) of the H3K27ac peaks downregulated in cKOs overlapped with a differential H3K79me2 peak, whereas the majority (99%) of upregulated H3K27ac peaks did not (FIG. 5B and FIG. 5C). From the 16,963 K79-REs identified by the ABC method, the vast majority (98%) did not show any difference in H3K27ac enrichment in cKOs, indicating that, at a genome-wide scale, H3K79me2 is not necessary for maintenance of H3K27ac in REs positive for both modifications (FIG. 10A). Despite corresponding to a small fraction (2%) of all K79-REs, those with differential H3K27ac made a relevant contribution to differential gene expression in Dot1L cKOs. From the 313 genes downregulated with H3K79me2 in gene body and K79-REs, 60 (19%) also had differential H3K27ac in at least one RE (FIG. 5D and FIG. 11A). From the 653 upregulated genes without gene body H3K79me2 but with K79-REs, 39% also had differential H3K27ac in at least one RE (FIG. 5E and FIG. 11A). As expected, for K79-REs associated with upregulated genes, differential H3K27ac peaks were mainly upregulated, whereas for K79-REs associated with downregulated genes, differential H3K27ac peaks were mainly downregulated (FIG. 11A). Altogether, these results revealed that, in mutants, differential H3K27ac within a subset of K79-REs may contribute to altered gene regulation by those REs, however, differential H3K27ac is not a prerequisite for gene regulation by K79-REs.

In summary, this data revealed that, during embryogenesis, DOT1L directly regulates critical cardiogenic transcription factors: Hand1, Irx4, Gata4, Tbx5, and Mef2c, all of which had gene body H3K79me2 combined with K79-REs (FIG. 5G and FIG. 11A). Both Hand1 and Irx4 were associated with at least one K79-RE with downregulated H3K27ac in Dot1L cKOs (FIG. 5E, FIG. 12G and FIG. 11A). Interestingly, Smyd1, encoding the H3K4 methyltransferase necessary for right ventricular expression of Hand2²², was also a direct target of DOT1L, unveiling complex epigenetic mechanisms for transcriptional regulation in cardiogenesis. Applicant's results also revealed that DOT1L has particular importance in maintenance of left ventricle-specific transcriptional networks, as it directly regulated not only transcription factors specifying the identity of this chamber (Hand1 and Tbx5), but also their direct downstream targets Cited1 and Gja5 (Cx40)²¹ (FIG. 5G). On the other hand, Nppa (ANF), another classic target of both HAND1²¹ and TBX5³³ was not altered in Dot1L cKOs, further highlighting the specificity and complexity of this epigenetic control (FIG. 5G). These findings reveal previously unrecognized roles for DOT1L and improve our understanding of the complex transcriptional and epigenetic landscape governing mammalian cardiogenesis.

### Impaired Cardiomyocyte Cell Cycle Withdrawal in Dot1L Mutants

Defective expression of core cardiogenic factors (including chamber-specific genes) can account for the abnormal morphology of Dot1L cKO hearts, however, it does not explain the increased wall thickness observed in postnatal mutant hearts (FIG. 1H). Dot1L cKO and Ctrl CMs had comparable sizes (FIG. 1I, FIG. 1J), therefore increased wall thickness in mutants was not caused by CM hypertrophy. As increased myocardial wall thickening coincided with the developmental period in which CMs withdraw from cell cycle²³, Applicant hypothesized this process might be affected in Dot1L cKOs. Flow cytometry analyses of cells isolated from P1 hearts revealed that, at this stage, about 50% of all cardiac cells were CMs (labeled by expression of the reporter gene tdTomato, FIG. 6A). Assessment of EdU incorporation rates (24 hours EdU pulse) revealed that, in control hearts, 19% of all CMs were proliferative, whereas this number increased to 26% in mutant hearts (FIG. 6A), representing a statistically significant increase in CM proliferation (FIG. 6B). In mice, by P10 most CMs are withdrawn from cell cycle and 80% of CMs are binucleated²³. Consistently, fluorescent microscopy analyses of CMs isolated from P10 hearts revealed that less than 3% of control CMs were EdU+ (24 hours EdU pulse, FIG. 6C and FIG. 6D), compared to 10% in mutant hearts (FIG. 6C and FIG. 6D). Nucleation analysis revealed that Dot1L cKO hearts had almost twice as many mononucleated CMs (34% in cKOs versus 19% in controls) at the expense of binucleated CMs (62% in cKOs versus 80% in Ctrls) (FIG. 6E). Quantification of proliferation ratios across different nucleation categories (FIG. 6F) revealed that Dot1L cKOs had a moderate increase in the percentage of EdU+ mononucleated CMs (8.1% in cKOs versus 5.2% in Ctrls) and a significant 4.9-fold increase in the percentage of EdU+ binucleated CMs (9.1% in cKOs versus 1.86% in Ctrls).

To validate these analyses, we assessed proliferation of control and Dot1L cKO CMs using the Rosa26-Fucci2a cell cycle reporter³⁴. Expression from this allele is Cre-dependent (thus, restricted to CMs in our analyses) and labels G1 CMs in red, and actively proliferating cells in yellow (G1/S) or green (S/G2/M)³⁴. Analysis of histological sections of P10 hearts (FIG. 6G, FIG. 6H and FIG. 13A and FIG. 13D) revealed that in controls the majority of CMs (67%) were in G1. G1/S CMs could also be detected (32%), but S/G2/M cells were rare (1%). On the other hand, Dot1L mutant CMs showed significantly different cell cycle distribution across all cycling phases: 34% in G1, 61% in G1/S and 5% in S/G2/M (FIG. 6G, FIG. 6H and FIG. 13A and FIG. 13D). In addition, increased proliferation of mutant CMs was also confirmed by phospho-Histone 3 (pH3) antibody staining in histological sections (FIG. 6G, FIG. 61 and FIG. 13B and FIG. 13C), as well as EdU analysis (2 hours EdU pulse, FIGS. 9D-9G) of Dot1L mutants and controls in the Rosa26-Fucci2a background. As left ventricle-specific transcripts were particularly affected in E16.5 Dot1L cKO hearts, we wondered whether a similar chamber tropism was operative for the neonatal proliferative phenotype.

Quantification of chamber-specific rates of proliferation (as assessed by Fucci2a reporter, pH3 or EdU incorporation, (FIG. 13A, FIG. 13B and FIG. 13F) revealed similar results in all compartments analyzed (RV, septum, LV, RA and LA), suggesting that the cell cycle phenotype is mechanistically distinct from the decreased expression of patterning genes observed during embryogenesis.

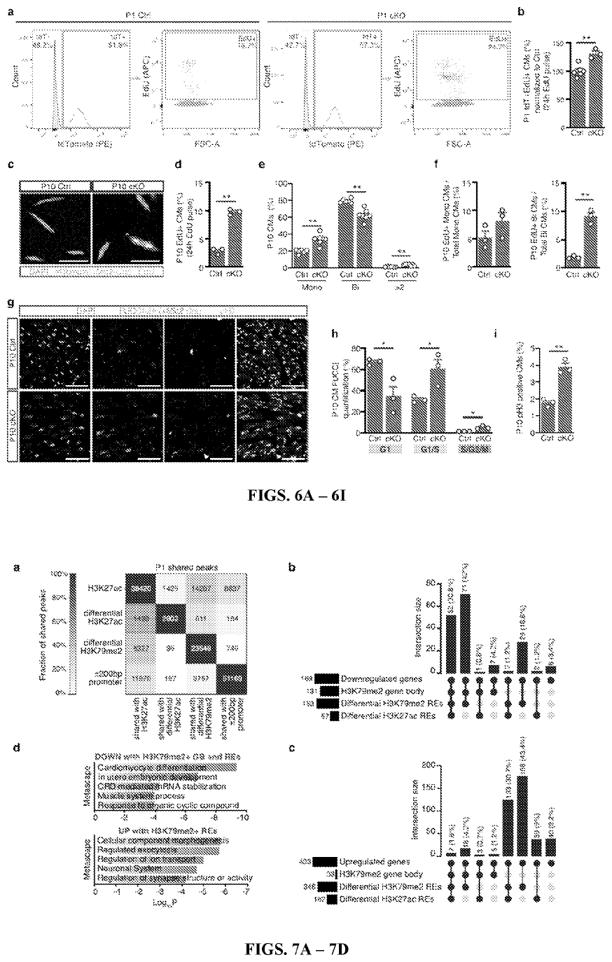

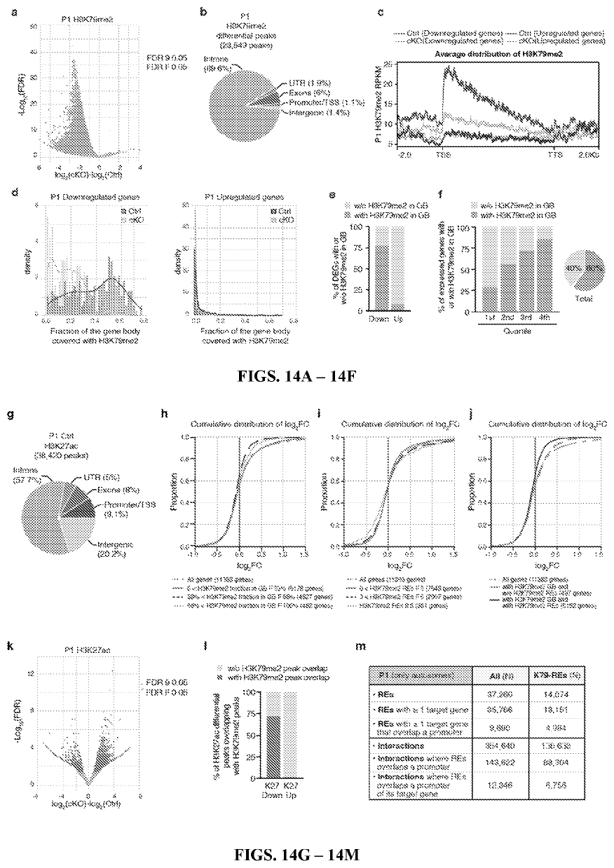

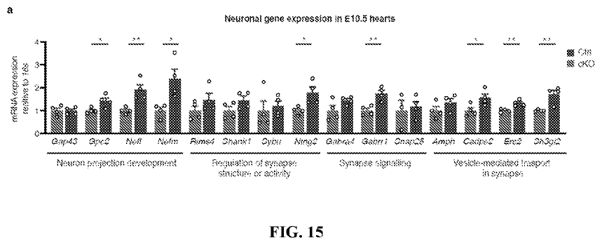

To gain insight into gene expression networks underlying the sustained proliferation of Dot1L cKO CMs, Applicant used neonatal (P1) FACS-sorted CMs to perform mechanistic analyses similar to those described for E16.5: RNA-seq, H3K79me2 and H3K27ac ChIP-seq, followed by bioinformatic analysis leveraging a publicly available CM Hi-C dataset³¹ and employing the ABC method to predict interactions between regulatory elements and their target genes (FIG. 7, FIG. 14 and FIG. 8B-FIG. 11B and FIG. 13B). These analyses revealed that, similar to E16.5, the majority of genes downregulated in cKO CMs (72.8%) were directly regulated by DOT1L via H3K79me2 both in gene body and REs (FIG. 7B). On the other hand, the vast majority of genes upregulated in cKOs (92.4%) did not have gene body H3K79me2, but 79.9% had interactions with K79-REs (FIG. 7C). Similar to what was observed in E16.5 CMs, from all K79-REs, the vast majority (96%) did not show altered H3K27ac signal in Dot1L cKO CMs (FIG. 10B). Differential H3K27ac was not a requirement for gene expression regulation by K79-REs as, within differentially expressed genes associated with K79-REs, 65% of downregulated genes and 60% of upregulated genes did not show differences in H3K27ac between genotype groups (FIG. 7B, FIG. 7C and FIG. 11B). Functional annotation revealed that multiple genes upregulated in cKOs and associated with K79-REs had important roles in the neuronal system and in synaptic structure or activity. qPCR analyses (FIG. 15) showed that genes in these categories were already upregulated in Dot1L cKO hearts in early cardiogenesis (E10.5), suggesting that their modulation is a direct consequence of DOT1L absence, rather than an indirect effect. On the other hand, genes downregulated in cKOs with H3K79me2 in gene body and REs were involved in CM differentiation (FIG. 7D and FIG. 12B). Within these was the gene encoding the cell cycle regulator p27 (Cdkn1b) (FIG. 7E). Interestingly, p27 knockouts have a phenotype of increased heart size due to defective CM cell cycle arrest that resembles the one of Dot1L cKOs³⁵. This observation strongly suggested that disrupted p27 expression accounts, at least in part, for the sustained CM proliferation observed in Dot1L cKOs (FIG. 711). In addition to p27, it is likely that this phenotype is also mediated by other upstream factors. To identify other factors potentially involved in the sustained proliferation observed in Dot1L cKOs, Applicant determined transcriptional regulators downregulated in Dot1L cKOs (FIG. 7F) and assessed transcription factor binding sites enriched in the K79-REs associated with genes downregulated in Dot1L cKOs with H3K79me2 in their gene body and in K79-REs (FIG. 7G). Together, these analyses suggested that TFs such as MEIS1 and MEIS2 might also play a role in the observed proliferative phenotype, which is consistent with known functions for these TFs in CM development post birth^{36,37}. Altogether these observations suggested that in the neonatal period DOT1L directly promotes expression of genes involved in CM maturation and cell cycle withdrawal whilst repressing expression of genes associated with non-CM functions.

### METHODS

### Mouse Strains and Experiments

Animal experiments were conducted according to protocols approved by the Institutional Animal Care and Use Committee at University of California, San Diego and the German local ethic committee (Regierungspräsidium Darmstadt, Hessen). All transgenic lines used were kept on an outbred background (Mus Musculus, Black-Swiss, Charles River laboratories). Mice were maintained in plastic cages with filtered air intake ports (Techniplast) on a 12 hr light cycle and have free access to water and food (Teklad LM-485 irradiated diet, Harlan Laboratories, catalog number 7912). All mouse housing rooms are maintained at 72+/−2 degrees Fahrenheit and 30-70% relative humidity. Adult (2-12 months old) males and females were used for breeding. For analyses conducted in embryonic stages, embryos were staged according to the embryonic day (E) on which dissection took place, with noon of the vaginal plug day being considered as E0.5 and birth typically occurring at E19. Experimental mice (males and females) were analyzed from embryonic day E10.5 to postnatal day 10. Dot1L^{fLOX} mice were obtained from the KOMP Repository (CSD29070) https://www.komp.org/geneinfo.php?geneid=54455. Floxed-out Dot1l^{Δ}mice were generated by crossing the Dot1l^{flox} allele with the epiblastic Meox2-Cre allele obtained from JAX laboratories (Stock No: 026858). xMlc2-Cre mice²⁴ were gently provided by Timothy Mohun. The Rosa26-tdTomato (Ai9) (tdTom) indicator allele²⁵ was purchased from JAX (Stock No: 007905). The Rosa26-Fucci2A cell cycle reporter allele was gently provided by Ian James Jackson³⁴. All experiments were performed using littermate cKOs and Ctrls. Images shown are representative examples of experiments with n≥3 biological replicates. In experiments assessing proliferation, mice received an injection of EdU (at P1 25 μL of a 3 g/L stock and at P10 50 μL of a 3 g/L stock) 2 h or 24 h prior to euthanasia.

For genotyping, genomic DNA was extracted by adding 250 μL of 50 mM NaOH to a tail tip biopsy and heating at 95° C. for 30 minutes. The solution was then neutralized by adding 50 μL of 1 μM Trish-HCl (pH 8.0). Genotyping PCR reactions (36 amplification cycles) were performed using Taq DNA Polymerase with ThermoPol Buffer (New England Biolabs, M0267L), dNTPs from Promega (U1511) and 1 μL of DNA solution. The following genotyping primers were used:

XMlc2Cre-Fw
5′-TAGGATGCTGAGAATCAAAATGT-3′;
XMlc2Cre-Rev
5′-TCCCTGAACATGTCCATCAGGTTC-3′;
Dot1L-Fw
5′-CCATATTAGTGTTCAAGGGCTACT-3′;
Dot1L-fl/wt-Rev
5′-AGCATAAGGATGCCAACTACTAAC;
Dot1L-null-Rev
5′-AAGGAGGTCCTACTCATAGTCCTT-3′;
Rosa26-tdTomato-Fw
5′-CTGTTCCTGTACGGCATGG-3′;
Rosa26-tdTomato-Rev
5′-GGCATTAAAGCAGCGTATCC-3′;
Rosa26-wt-Fw
5′-AAGGGAGCTGCAGTGGAGTA-3′;
Rosa26-wt-Rev
5′-CCGAAAATCTGTGGGAAGTC-3′;
Rosa26-wt-Fw
5′-CAAAGTCGCTCTGAGTTGTTATCAG-3′;
Rosa26-wt-Rev
5′-GGAGCGGGAGAAATGGATATGAAG-3′;
Rosa26-Fucci2a-Rev
5′-TCACCCAGGAGTCATTTGAT-3′.

### Echocardiography and Electrocardiography

Echocardiography was performed at P5 and P10 using a Vevo3100 imaging system (VisualSonics) with MX550 (for P10) or MX700 (for P5) probes. Two-dimensional-guided M-mode images of the short axis at the papillary muscle level were recorded. Data were analyzed by VevoLab 3.2.5 software using Auto-LV technology to minimize the variability between the measurement and confirmed by an experienced researcher. For electrocardiography (ECG) measurements the PR interval was measured from the beginning of the P-wave to the beginning of the QRS complex; QRS duration was measured from the first deflection of the Q-wave to the nadir of the S-wave (defined as the point of minimum voltage in the terminal portion of the QRS complex); the R-R interval was obtained as the average R-R interval over the sampling period.

### Immunohistochemistry

Tissues were isolated in cold PBS and fixed in 4% paraformaldehyde at 4° C. overnight. Tissues were dehydrated in a sucrose gradient (5% for 1 hour, 12% for 1 hour, 20% overnight) and embedded in a 1:1 mix of 20% Sucrose and Tissue-Tek Optimal Cutting Temperature compound (OCT, Sakura). 10 μm thick histological sections were cut using a cryostat (Leica CM3050) and kept at −20° C. (short-term) or −80° C. (long-term) until being processed for immunofluorescence. Sections or isolated CMs were blocked in 10% donkey serum before incubation with antibodies. Primary antibodies were incubated at 4° C. overnight. The following primary antibodies were used: anti-a Sarcomeric Actinin (1:400 Abcam #ab68167), anti-Myomesin (1:200 mMaC #B4 developed by Perriard, J.-C. was obtained from the Developmental Studies Hybridoma Bank, created by the NICHD of the NIH and maintained at The University of Iowa), anti-Vimentin (1:100 Abcam #ab45939), anti-TNNT (1:100 Thermo Fisher #MA5-12960), anti-PDGFR-a (1:200 R&D Systems #AF1062), anti-Collagen 1 (1:200 Abcam #ab34710), anti-tdTomato (1:100 Sicgen #ab8181-200), anti-GFP (1:600 Abcam #ab13970), anti-phosphoH3 (1:100 Millipore #06-570). EdU incorporation was detected using a Click-iT® EdU kit (Thermo Fisher Scientific; #C10340). Secondary antibodies were incubated at room temperature for 1 hour and 30 minutes. The following secondary antibodies were used at a concentration of 1:400: donkey anti-rabbit Alexa fluor 647 (Thermofisher #A31573), donkey anti-goat Alexa fluor 488 (Thermofisher #A11055), donkey anti-goat Alexa fluor 555 (Thermofisher #A21432), donkey anti-chicken Alexa fluor 488 (Jackson Immuno Research #703-545-155), donkey anti-rabbit Alexa fluor 555 (Thermofisher #A31572), donkey anti-mouse Alexa fluor 488 (Thermofisher #A21202), donkey anti-goat Alexa fluor 647 (Thermofisher #A21447). Masson Trichrome staining of tissue sections was performed using the Masson trichrome stain kit (Sigma, HT15), following instructions provided by the manufacturer. Immunofluorescence images were acquired using a Keyence BZX-700 fluorescent microscope, an Olympus FV1000 or a Leica SP8 scan confocal microscope. Bright field images were acquired using a Nikon Eclipse C1 microscope. Image processing was performed with Fiji and Volocity software.

### Rna Scope

RNAscope fluorescent in situ hybridizations (ISH) were conducted using the RNA-scope Multiplex Fluorescent Reagent Kit v.2 (Advanced Cell Diagnostics, 323100) following standard protocols provided by the manufacturer, using the following RNA-scope probes (ACDbio): Mm-Dot1L-C2 (#533431-C2); Mm-Hand1-C2 (#429651-C2); Mm-Irx4 (#504831); Mm-Hand2 (#499821); Mm-Smyd1-C3 (#1152831-C3).

### Cardiomyocyte Isolation

Embryonic and neonatal CMs were isolated using a modified version of the previously described protocol⁴¹. Embryonic or postnatal day 1 hearts were isolated and transferred into ice cold HBSS. Embryonic single cell suspensions from were obtained by performing eight rounds of enzymatic digestion (5 minutes each) with Collagenase type II (0.7 mg/ml Worthington) at 37° C. under agitation. Postnatal day 1 single cell suspensions were obtained by performing an overnight digestion with trypsin (0.5 mg/ml) at 4° C. followed by eight rounds of enzymatic digestion (5 minutes each) with Collagenase type II (1 mg/ml Worthington) at 37° C. Cells were collected in cold medium containing fetal calf serum to stop the enzymatic reaction and centrifuged at 210 rcf to allow initial separation of CMs from other cardiac cells. Cell preps were resuspended in FACS buffer (HBSS, 5% FBS, 2.5 mM EDTA) and incubated for 30 minutes with APC-conjugated, FACS-validated antibodies against CD31 (endothelial cells, clone: MEC13.3; BioLegend #102510), CD45 (leukocytes, clone: 30-F11, BioLegend #103112), CD140a (Fibroblasts, clone: APA5, eBioscience #17-1401-81) and TER119 (erythroid cells, clone: TER-119, BioLegend #116212) to avoid doublets between CMs and stromal cells. Live/Dead (Invitrogen #L34957) or DAPI staining was performed to exclude dead cells. Embryonic and postnatal day 1 live and single control and mutant CMs were sorted based on the red fluorescence emitted by the Cre reporter tdTomato using a FACS Aria II or Influx Cell sorter (BD Biosciences) and collected in TRIZol reagent for RNA extraction or cross-linked as described below for chromatin analysis.

Postnatal day 10 CMs were isolated using a Langendorff system using Collagenase type II (0.7 mg/ml Worthington). After perfusion cells were dissociated from the heart, collected in conical tubes and allowed to settle by gravity in order to obtain separation of viable, rod-shaped CMs from dead CMs and from interstitial cells of the heart. For histological analysis, isolated CMs were fixed in 4% PFA and processed for immunostaining.

### Protein Isolation and Western Blot Analysis

Total protein extracts were prepared by lysing samples in RIPA buffer. Protein lysates in Laemmli buffer were separated by electrophoresis on 12% SDS-PAGE gels and transferred for 2 hours at 4° C. on to a PVDF membrane (BioRad). After blocking for an hour in 5% dry milk, membranes were incubated overnight at 4° C. with the primary antibody in blocking buffer. The following primary antibodies were used: H3K79me2 (1:1000 abcam #ab3594) and anti-H3 (1:5000 abcam #ab1791). Blots were washed and incubated with a horseradish peroxidase (HRP)-conjugated secondary antibody generated in Rabbit (1:2000; Cell Signaling Technology #7074) for 1.5 hours at room temperature. Immunoreactive protein bands were visualized using an enhanced chemiluminescence reagent (Thermo Fisher Scientific). Protein quantification was achieved using ImageJ software.

### Quantification of Proliferation by Flow Cytometry

FACS quantification of rates of EdU incorporation was carried out using littermate Dot1L cKOs and Ctrls. EdU detection was done in cell suspensions using the Click-iT® EdU Alexa 647 kit (Thermo Fisher Scientific; C10340), according to the manufacturer's instructions. TdTomato signal was used to discriminate CMs from other lineages of cardiac cells. Stained cells were analyzed using a FACS Canto II flow cytometer (BD Bioscience). DIVA (v8.0.1) and FlowJo (v10.8.1) software (BD Pharmingen) were used for data acquisition and analysis.

Rna Extraction, qRT-PCR and RNA-Seq.

RNA was extracted using Tryzol (Invitrogen #15596026) and Direct-zol RNA Kits (Zymo Research #R2061) following instructions provided by the manufacturers. All transcriptomics analyses (qRT-PCR or RNA-seq) were performed on FACS-sorted CMs, except qPCR in Supplementary FIG. 3a and Supplementary FIG. 8 that were performed on whole heart tissue. For E16.5, P1, P5 and P10 biological replicates were prepared from single hearts. For E12.5, preparation of each biological replicate required pooling of hearts with same genotypes. For each stage analyzed, a minimum of 3 biological replicates, prepared from littermate Dot1L Ctrl and Dot1L cKO hearts, were used. For qRT-PCR experiments, cDNA was produced using the SuperScript VILO cDNA Synthesis Kit (Invitrogen #11754050). qRT-PCR was performed using SYBR Select Master Mix for CFX (Applied biosystems #4472952) on a Bio-Rad CFX96 Real-Time PCR system or Perfecta SYBR green FastMix (VWR #733-1389) on an Applied Biosystems ViiA7 Real-Time PCR. The following primers were used:

Dot1L-Ex2-Fw
5′-TGCTGCTCATGAGATTATTGAGA-3′ (primer hybridizing
within the loxP-flanked exon2 of the floxed Dot11
allele);
Dot1L-Ex4-Rev
5′-ATGGCCCGGTTGTATTTGTC-3′;
18s-Fw
5′-AAATCAGTTATGGTTCCTTTGGTC-3′;
18s-Rev
5′-GCTCTAGAATTACCACAGTTATCCAA-3′;
Col1a1-Fw
5′-CATGTTCAGCTTTGTGGACCT-3′;
Col1a1-Rev
5′-GCAGCTGACTTCAGGGATGT-3′;
Col3a1-Fw
5′-ACGTAGATGAATTGGGATGCAG-3′;
Col3a1-Rev
5′-GGGTTGGGGCAGTCTAGTG-3′.
Gap43-Fw
5′-ATAACTCCCCGTCCTCCAAGG-3′
Gap43-Rev
5′-GTTTGGCTTCGTCTACAGCGT-3′
Gpc2-Fw
5′-CTGCCCGGCATAGAAAGTTTA-3′
Gpc2-Rev
5′-GCGACCATAGGAATGCGAGAA-3′
Nefl-Fw
5′-TGATGTCTGCTCGCTCTTTC-3′
Nefl-Rev
5′-CTCATCCTTGGCAGCTTCTT-3′
Nefm-Fw
5′-ACAGCTCGGCTATGCTCAG-3′
Nefm-Rev
5′-CGGGACAGTTTGTAGTCGCC-3′
Rims4-Fw
5′-CTACTTCCCGTGCATGAACTC-3′
Rims4-Rev
5′-CCTCCATAGTTAAGGTTGCCCT-3′
Shank1-Fw
5′-TGCATCAGACGAAATGCCTAC-3′
Shank1-Rev
5′-AACAGTCCATAGTTCAGCACG-3′
Sybu-Fw
5′-GCGATGAAGACTTTACCAGGAA-3′
Sybu-Rev
5′-CCTCGGTTGCGTGAGAAAGA-3′
Ntng2-Fw
5′-GTGATGCGCCTGAAGGATTAT-3′
Ntng2-Rev
5′-TTCTCATGGGAACAGAACCTTTC-3′
Gabra4-Fw
5′-ACAATGAGACTCACCATAAGTGC-3′
Gabra4-Rev
5′-GGCCTTTGGTCCAGGTGTAG-3′
Gabrr1-Fw
5′-CGAGGAGCACACGACGATG-3′
Gabrr1-Rev
5-GTGAAGTCCATGTCAACCTCTG-3′
Snap25-Fw
5′-CAACTGGAACGCATTGAGGAA-3′
Snap25-Rev
5′-GGCCACTACTCCATCCTGATTAT-3′
Amph-Fw
5′-TCCGGGGATATTTAGCAGCAA-3′
Amph-Rev
5′-TGGCTCGTAGACTTCATGTAGAG-3′
Cadps2-Fw
5′-CTTGGTTGTCCGCTACGTTGA-3′
Cadps2-Rev
5′-GTTGAGCCATTGTTGACAGGC-3′
Erc2-Fw
5′-AAAGCAGCAGACCCAGAACA-3′
Erc2-Rev
5′-TGGTGGTGGTGGTAATGGTG-3′
Sh3g12-Fw
5′-AACGATTGAATACCTCCAACCC-3′
Sh3g12-Rev
5′-TTCACTTCCATGTCCAATGAGTC-3′

For RNA-seq experiments libraries were generated from 25 ng of RNA using the TruSeq Stranded mRNA library Prep kit (Illumina #20020594) and sequenced on a HiSeq 4000 System (Illumina) using a single read 50 protocol.

### Chip-Seq

ChIP-seq was essentially performed as described42,43. Briefly, cells were fixed in in 1% formaldehyde/PBS for 10 minutes at room temperature, the reactions quenched by adding 2.625 μM glycine to 125 mM final 20% BSA to 0.5% final and cells pelleted by centrifugation for 5 minutes at 1,000 g, 4° C. Cells were washed twice with ice-cold 0.5% BSA/PBS and cell pellets were snap-frozen in liquid nitrogen and stored at −80° C. For H3K79me2 ChIP-seq fixed cells were thawed on ice, resuspended in 500 μl ice-cold buffer L2 (0.5% Empigen BB, 1% SDS, 50 mM Tris/HCl pH 7.5, 1 mM EDTA, 1×protease inhibitor cocktail) and chromatin was sheared to an average DNA size of 300-500 bp by administering 7 pulses of 10 s duration at 13 W power output with 30 s pause on wet ice using a Misonix 3000 sonicator. The lysate was diluted 2.5-fold with ice-cold L2 dilution buffer (20 mM Tris/HCl pH 7.4@20° C., 100 mM NaCl, 0.5% Triton X-100, 2 mM EDTA, 1×protease inhibitor cocktail), and one percent of the lysate was kept as ChIP input. For each immunoprecipitation, aliquots of diluted lysate equivalent to 150,000 to 1 million cells, 20 μl of Dynabeads Protein A (for rabbit polyclonal antibodies) and 2 g anti H3K79me2 antibody (Abcam #ab3594) were combined and rotated overnight at 8 RPM and 4° C. For H3K27ac ChIP-seq fixed cells were thawed on ice, resuspended in 100 μl ice-cold RIPA buffer (50 mM Tris-HCl pH 7.5, 150 mM NaCl, 1% IGEPAL CA-630 0.25% Na-Deoxycholate, 0.1% SDS, 1 mM EDTA, 1×protease inhibitor cocktail). Chromatin was sheared for 90 minutes on an Active Motif PIXUL high-throughput sonicator using standard settings (Pulse [N]: 50, PRF:1 kHz, Burst Rate: 20 Hz). Two microliter of each lysate was kept to generate ChIP input libraries, and the remainder used to immunoprecipitate H3K27ac-associated DNA by adding 2 g anti-H3K27ac antibody (Active Motif 39133, lot 31521015) and 20 μl of Dynabeads Protein A (Thermo) and rotating overnight at 8 RPM and 4° C. The following day, in the case of either H3K79me2 or H3K27ac ChIP-seq, beads were collected on a magnet and washed three times with 150 μl each of ice-cold wash buffer I (10 mM Tris/HCl pH 7.5, 150 mM NaCl, 1% Triton X-100, 0.1% SDS, 2 mM EDTA), wash buffer III (10 mM Tris/HCl pH 7.5, 250 mM LiCl, 1% IGEPAL CA-630, 0.7% Deoxycholate, 1 mM EDTA) and twice with ice-cold TET (10 mM Tris/HCl pH7.5, 1 mM EDTA, 0.2% Tween-20). Libraries were prepared directly on the antibody/chromatin-bound beads: after the last TET wash, beads were suspended in 25 μl TT (10 mM Tris/HCl pH7.5, 0.05% Tween-20), and libraries were prepared using NEBNext Ultra II reagents according to the manufacturer's protocol but with reagent volumes reduced by half, using 1 μl of 0.625 μM Bioo Nextflex DNA adapters per ligation reaction. DNA was eluted, proteins digested and crosslinks reversed by adding 4 μl 10% SDS, 4.5 μl 5 μM NaCl, 3 μl EDTA, 1 μl proteinase K (20 mg/ml), 20 μl water, incubating for 1 h at 55° C., then overnight at 65° C. DNA was cleaned up by adding 2 μl SpeedBeads 3 EDAC (Cytiva) in 61 μl of 20% PEG 8000/1.5 μM NaCl, mixing and incubating for 10 minutes at room temperature. SpeedBeads were collected on a magnet, washed twice by adding 150 μl 80% EtOH for 30 s each, collecting beads and aspirating the supernatant. After air-drying the SpeedBeads, DNA was eluted in 25 μl TT and the DNA contained in the eluate was amplified for 12 cycles in 50 μl PCR reactions using NEBNext High-Fidelity 2X PCR Master Mix or NEBNext Ultra II PCR master mix and 0.5 μM each of primers Solexa IGA and Solexa 1 GB. Libraries were cleaned up as above by adding 36.5 μl 20% PEG 8000/2.5 μM NaCl and 2 μl SpeedBeads, two washes with 150 μl 80% EtOH for 30 s each, air-drying beads and eluting the DNA into 20 μl TT. ChIP library size distributions were estimated following 2% agarose/TBE gel electrophoresis of 2 μl library, and library DNA amounts measured using a Qubit HS dsDNA kit on a Qubit fluorometer. ChIP input material (1 percent of sheared DNA) was treated with RNase for 15 min at 37° C. in EB buffer (10 mM Tris pH 8, 0.5% SDS, 5 mM EDTA, 280 mM NaCl), then digested with Proteinase K for 1 h at 55° C. and crosslinks reversed at 65° C. for 30 min to overnight. DNA was cleaned up with 2 μl SpeedBeads 3 EDAC in 61 μl of 20% PEG 8000/1.5 μM NaCl and washed with 80% ethanol as described above. DNA was eluted from the magnetic beads with 25 μl of TT and library prep and amplification were performed as described for ChIP samples. For H3K79me2 ChIP-seq, libraries were sequenced single-end for 75 cycles (SE75) to a depth of 20-25 million reads, for H3K27ac ChIP-seq libraries were sequenced single-end for 76 cycles (SE76) to a depth of 15-22 million reads on an Illumina NextSeq 500 instrument.

### Bioinformatic Analyses

### RNA-Seq

Sequencing reads were processed to remove Illumina barcodes and aligned to the UCSC Mus musculus reference genome (build mm10) using STAR v.2.5.1b with default parameters44. ReadsPerGene.out.tab files were then processed with edgeR45. RNA expression was calculated in reads per kilobase per million mapped reads (RPKM) considering the sum of exon length. Differentially expressed coding genes were selected based on the following parameters: FDR≤0.05, RPKM≥1 in at least one biological condition, and log 2 Fold Change≤−0.5 for genes downregulated and ≥0.5 for genes upregulated in Dot1L cKOs vs Dot1L Ctrls. Pathway analysis was performed using METASCAPE⁴⁶.

### Chip-Seq Alignment and Peak-Calling

All analyses were performed using the mouse reference genome GRCm38 (mm10) and the gencode gene annotation version vM25. H3K79me2 and H3K27ac ChIP-seq data was processed in the same manner. Bowtie2⁴⁷ was applied to align the fastq files to the mouse reference genome. First, the required index structure was built using: bowtie2-build-f—seed 123—threads 20Mus_musculus.GRCm38.dna.primary assembly.fa mouse_GRCM38_mm10. To get the alignment files we ran: bowtie2-x mouse_GRCM38_mm10-U<fastq-file>-S output-file-name>.sam-q-t-p 30. Conversion of the resulting files (sam) to bam format was done using samtools (version samtools 1.10)⁴⁸. To allow easy visualization in a genome browser, bam files were converted to bigWig (bw) files using deeptools bamCoverage function. Next, peak-calling was performed with MACS2 (version macs2 2.2.7.1)⁴⁹: macs2 callpeak-t<treatment>.bam-c<input>.bam-n<prefix-name>—outdir<output-dir>-f BAM-g 1.87e9-B, where <treatment> is either the aligned reads of the Dot1L Ctrl or Dot1L cKO and <input> the corresponding input signal. For all following analyses we used the narrowPeak files. To compute differential ChIP-seq sites between Ctrl and Dot1L cKOs, we applied DiffBind (version 2.10.0 for H3K79me2 and version 3.4.11 for H3K27ac bioconductor-diffbind)⁵⁰.

### Determining Genomic Distribution of H3K79Me2 or H3K27Ac Peaks

To determine the location of peaks in relation to distinct genomic regions, the gene annotation was split into exons, UTRs and gene bodies. Promoter regions of 400 bp length, centered around the most 5′ TSS, were added. All those genomic regions were made exclusive, so that each base pair of a gene had one unique label. The remaining base pairs located within gene bodies but not overlapping any other feature were labelled as introns. Based on those annotations, we determined the location of each base pair of each ChIP-seq peak, using bedtools (v2.25.0)51, and visualized their distribution with pie charts. ChIP-seq tracks were visualized with Integrative Genomics Viewer (IGV) (v2.9.2).

### Computing Coverage and Fraction of the Gene Body Covered by H3K79Me2

The profiles of the H3K79me2 signal at the gene body were computed using deeptools⁵². We used the bam-files resulting from the bowtie2 analysis and the up- and downregulated genes in bed-file format (with strand information).

First, Applicant applied deeptools bamCompare to determine the mean signal of the two replicates of Ctrl and cKO for each time point: bamCompare-b1<treatment repl>.bam-b2<treatment rep2>.bam-o<treatment>_mean_rep1_rep2.bw-of bigwig—scaleFactorsMethod None—operation mean—effectiveGenomeSize 2652783500-p 25—normalizeUsingRPKM—binSize 20—skipNonCoveredRegions, where <treatment> is either Ctrl or cKO.

Next, Applicant visualized the data using deeptools computeMatrix and plotProfile functionalities: computeMatrix scale-regions-S Ctrl mean_rep1_rep2.bw cKO mean_rep1_rep2.bw-R downregulated genes FDR≤0.05.bed upregulated genes FDR_0.05.bed-o inputProfile.mat.gz—endLabel TTS—beforeRegionStartLength 2000—afterRegionStartLength 2000—regionBodyLength 5000-p 20-skipZeros

plotProfile-m inputProfile.mat.gz-out profile.pdf—perGroup—startLabel TSS—endLabel TTS—plotTitle “Average distribution of H3K79me2 relative to the distance from TSS and TTS”—samplesLabel “Ctrl (upregulated genes)” “cKO (upregulated)”—regionsLabel “downregulated genes” “upregulated genes”—plotFileFormat pdf

To compute the fraction of the gene body which is covered by H3K79m2, Applicant applied bedtools (bedtools v2.25.0) coverage function. Given a set of genomic regions and a bam-file, the function computes the number of reads that overlap each genomic region and the fraction of bases that have a non-zero coverage based on the bam-file. Applicant determined the number of overlapping reads and the fraction for all annotated mouse genes for Ctrl and cKO. Next, the mean fraction (of rep1 and rep2) of the up- and downregulated genes was computed. The density plots are based on all up- and downregulated genes.

Calling Regulatory Interactions with H3K27Ac ChIP-Seq and Hi-C

For the identification of enhancer regions H3K27ac ChIP-seq was performed on FACS sorted CMs at E16.5 and P1. A Hi-C matrix of mouse CMs was downloaded from GEO (GSM2544836)³¹. The Hi-C matrix was normalized using Knight-Ruiz normalization with the Juicebox dump command (version 1.22.01)⁵³ with a resolution of 5000 bp for each chromosome.

java-jar juicer tools 1.22.01.jar dump observed KR<hic-file>chr$ chr$ BP 5000<output-file-name>

The regulatory interactions between promoter and candidate enhancers were assessed with an adapted version of the ABC-score of Fulco et al.²⁷ from the STARE software²⁸. The command was as follows:

STARE ABCpp-b<H3K27ac-peak-file>-n 7-f<folder-with-HiC-files>-k 5000-t 0.02-a gencode.vM25.annotation.gtf-w 5000000-o<output-path>

For all genes on the autosomes (GRCm38p6) the most 5′ TSS was taken and all candidate enhancers in a 5 Mb window centered on the TSS were scored according to the following equation:

$$\overline{ABC} {score}_{e , g} = \frac{{A}_{e , g} \cdot {C}_{e , g}}{\sum i ∈ {E}_{g} {A}_{i , g} \cdot {C}_{i , g}} \qquad Equation 1 )$$

The interaction of an enhancer (e) with a gene (g) is described by the gene-specific activity of the enhancer (Ae,g) and the contact to the gene (Ce,g). The adapted ABC-score returns the relative contribution of an interaction in relation to all other candidate interactions of that gene (Eg). The gene-specific activity of an enhancer is defined as follows:

$${A}_{e , g} = {A}_{e} \frac{{C}_{e , g}}{\sum j ∈ {C}_{e} {C}_{e , j}} \qquad Equation 2 )$$

Where the activity of an enhancer (Ae), measured as signal of the H3K27ac ChIP-seq peak, is taken relative to the contacts that an enhancer has to its candidate target genes (Ge). The contact between an enhancer and a gene's TSS is the contact frequency of the respective bins in the normalized Hi-C matrix. All candidate enhancer-gene interactions with an adapted score≥0.02 were used for further analyses. Chromosomes X and Y were excluded from the analysis. Statistical differences between the cumulative log 2(fold-change of gene expression) distributions were calculated pairwise with a two-tailed Kolmogorov-Smirnov test (SciPy 1.4.1, stats module, ks_2samp function).

### Mapping Differential ChIP-Seq Signal to Genes

An RE was considered differential for H3K27ac/H3K79me2 if ≥10% of the enhancer's length was covered by a differential ChIP-seq signal.

The REs differential for H3K27ac were mapped to genes via the adapted ABC-score. If a RE had a significant change in H3K27ac signal in cKO versus Ctrl, it was accounted for all genes that had an ABC interaction to that RE in Ctrl. Additionally, we considered H3k27ac signal changes in all REs present only in cKO that were mapped to a gene via ABC interactions in cKO but not in Ctrl.

### Motif Enrichment Analysis

To identify transcription factors likely regulating the activity of K79-REs associated to genes differentially expressed in Dot1L cKOs, a motif enrichment analysis was performed. To this end, K79-REs associated to differentially expressed genes were divided into two groups: those associated with genes downregulated in Dot1L cKOs with H3K79me2 in their gene body (activating K79-REs) and those associated with genes upregulated in Dot1L cKOs without H3K79me2 in their gene body (silencing K79-REs). TF binding motifs (total of 515) were downloaded from the JASPAR database⁵⁴ and only those significantly expressed (RPKM>1) in E16.5 or P1 CMs were considered in subsequent analyses. Using TRAP⁵⁵, a TF-affinity value per TF for each RE sequence was computed. The value is defined as the sum over all binding site probabilities of the given TF for the current sequence. The TRAP analysis was performed separately for the RE sequences of the up- and downregulated genes. A one-tailed Mann-Whitney test (using R's Wilcox test function, confidence level of 0.975) to identify TFs enriched in the activating K79-REs versus silencing K79-REs and vice versa was performed. The resulting p-values were adjusted by applying the Benjamini-Hochberg procedure⁵⁶. All TFs with an adjusted p-value of <0.05 were considered significant.

### Pathway Enrichment Analysis

Pathway enrichment analysis of genes downregulated with H3K79me2 in gene body and regulatory elements and genes upregulated with H3K79me2 in regulatory elements but not H3K79me2 in gene body was performed using Metascape web tool⁴⁶ using standard settings.

### Quantification of Experiments, Statistical Analysis and Reproducibility

CM cell length and width and RNA-scope puncta were measured using Image J. Statistical significance of differences in the survival of DOT1L mice was assessed using Kaplan-Meier survival analysis with the log-rank method of statistics. In FIG. 2 graphs data are expressed as mean±SD, in all other graphs, data are expressed as mean±SEM, with a minimum of 3 biological replicates (the exact replicate number is described in the legend to each FIG.). Statistical significance of differences among groups was tested by 2-tailed Student's t-test. A value of P≤0.05 was considered statistically significant. * represents P≤0.05, **P≤0.01. Analyses were performed with GraphPad Prism software. Each experiment was repeated independently with similar results at least 2-3 times.

### DISCUSSION

Devising a blueprint for transcriptional and epigenetic mechanisms regulating normal heart development is of critical importance for understanding congenital heart disease and to pave the way for regenerative therapies of the adult heart post injury. Previous studies using embryonic stem cells led to the hypothesis that DOT1L might play a relevant role in cardiogenesis, which was confirmed with the in vivo experiments reported here. In addition, Applicant deciphered previously unrecognized mechanisms of action of this epigenetic modifier. Dot1l W is ubiquitously expressed in the heart and its CM-specific conditional ablation resulted in a fully penetrant phenotype of an enlarged and rounded heart that culminated in neonatal lethality (FIG. 1 and FIG. 2). Genome-wide transcriptomic and ChIP-seq assays provided detailed mechanistic insight into gene expression programs underlying this phenotype, highlighting DOT1L as a direct regulator of two processes that take place at distinct developmental stages: expression of critical cardiogenic factors in embryogenesis (FIG. 3, FIG. 4 and FIG. 5) and neonatal CM maturation and cell cycle withdrawal (FIG. 6 and FIG. 7). Detailed bioinformatic analyses revealed that DOT1L regulated expression of its targets via methylation of H3K79 in gene body, regulatory regions, or a combination of both. Changes in H3K27ac profiles were observed in a significant number of K79-REs associated with genes differently expressed in Dot1L cKOs, but were not a requirement for gene expression regulation by K79-REs.

In embryogenic stages, DOT1L directly regulated several genes involved in defining distinct cardiac chambers. Amongst these, there was a clear enrichment in genes required for left ventricular identity (Hand1, Tbx5, Cited1, Gja5). From these, Hand1 emerged as a gene particularly sensitive to absence of DOT1L activity. Likely reflecting this tight regulation, the dilated and rounded heart phenotype with neonatal lethality displayed by Dot1L mutants strongly resembles the phenotype of Hand1 conditional mutants²¹. How disruption of a left ventricle-specific transcription factor results in a rounded heart morphology is not currently clear and addressing this question will require a full understanding of gene expression networks downstream of HAND1. Hand1 cardiac cKOs also display ventricular septal and outflow defects, whereas Dot1L mutants do not. This is likely a consequence of residual Hand1 transcripts at earlier embryonic stages—Hand1 transcripts were completely absent from E16.5 Dot1L cKO CMs but could be detected at E10.5 (although at much lower levels than in stage-matched controls, FIG. 3). Interestingly, postnatally Hand1 is restricted to the cardiac conduction system and disrupting this specific expression causes conduction system problems¹⁸. Therefore, it is conceivable that electrocardiographic anomalies observed in Dot1L cKOs are, at least in part, caused by blunted Hand1 expression. The dependence of Hand1 expression on DOT1L is of particular relevance for our understanding of epigenetic regulation of cardiogenesis. Previous studies have shown that SMYD1 (that catalyzes H3K4 methylation) is critical for activation of Hand2²², but an epigenetic mechanism specifically regulating Hand1 has remained elusive. Together with these previous observations, our results suggest a model in which DOT1L and SMYD1, two enzymes expressed throughout the heart, are essential for maintenance of normal levels of asymmetrically expressed cardiogenic factors (FIG. 5G).

DOT1L depletion resulted in absence of Hand1 expression without affecting expression of Hand2, whereas absence of SMYD1 results in blunted activation of Hand2 without affecting expression of Hand1²². DOT1L also regulated expression of Smyd1 itself, revealing a complex epigenetic mechanism controlling normal cardiogenesis. It is unclear if SMYD1 is involved in regulating Dot1l W expression. Interestingly, despite the fact that Smyd1 transcripts were significantly downregulated in Dot1L cKOs, its critical target Hand2 was not altered in these mutants, suggesting that the levels of SMYD1 protein present in Dot1L cKOs are sufficient to sustain mechanisms of transcriptional regulation dependent on this enzyme. Despite regulating distinct Hand genes, DOT1L and SMYD1 also share some common targets. For example, transcription of Irx4 is dependent on the action of both enzymes (see Applicant's disclosed data and²²). IRX4 is required for ventricular myocyte identity, and it is conceivable that DOT1L and SMYD1 act in a coordinated way to ensure a rheostatic control of Irx4 transcription. How DOT1L and SMYD1, two enzymes that are expressed throughout the heart, achieve regulation of chamber-specific genes (ventricular versus atrial, left versus right) is not currently known and will be the subject of future studies. Given that several of DOT1L targets are also targeted by critical regulators of cardiogenesis, such as NKX2.5 and TBX5, it is possible that the specificity of these epigenetic enzymes is derived from their interactions with distinct transcription factors.

Initial studies suggested that H3K79 methylation is a generic marker of active transcription⁵, but Applicant's datasets clearly revealed that, in CMs, H3K79me2 methylation is not a basic requirement for gene transcription, as in control animals, multiple genes are active without having gene body H3K79 methylation. From the group of genes that bear this modification in controls and lose it in mutant hearts, two major categories emerge: those that are modulated upon DOT1L loss and those that are unaffected by the absence of this enzyme. The latter may reflect mechanisms of epigenetic redundancy. Applicant's bioinformatic analyses integrating differential H3K79me2 ChIP-seq peaks with a mark of regulatory regions³⁸ (CM H3K27ac ChIP-seq) and 3-dimensional genomic conformation (CM Hi-C) suggested a model in which, in addition to gene body H3K79me2, DOT1L mediates expression of target genes via H3K79me2 in cis-regulatory elements. In loci with gene body H3K79me2, those associated with K79-REs were more downregulated in Dot1L cKOs. The magnitude of downregulation correlated with two variables: extent of H3K79me2 gene body coverage and the number of K79-REs interacting with the gene, suggesting these two forms of H3K79me2 synergize to promote expression of target genes. Interestingly, Applicant's analyses suggested that K79-REs could also be of an inhibitory nature, blocking transcription of target genes that lack gene body H3K79me2. To the best of Applicant's knowledge this is the first report of an involvement of DOT1L/H3K79me2 in this form of gene expression regulation, however, it is also conceivable that gene upregulation observed in Dot1L cKOs reflects secondary effects (for example, downregulation of a transcription regulator with repressor properties).

In mice, after the first week of life, most CMs are binucleated and completely withdrawn from the cell cycle²³. Resistance of mature CM to proliferation is a major hurdle for cardiac regeneration^{39,40}. Thus, devising strategies to promote reacquisition of proliferative potential in CMs is a major priority in cardiac regenerative medicine^{39,40}. FACS and histological studies using biochemical and genetic strategies to identify CM-specific proliferation revealed that Dot1L cKO CMs retain proliferative potential at P10, a stage at which control counterparts are already withdrawn from the cell cycle (FIG. 6). Notably, Dot1L cKO hearts also had a higher percentage of mononucleated CMs than control hearts. Transcriptome analyses suggested that DOT1L depleted CMs are less mature than control CMs and fail to activate expression of p27, a known repressor of cell cycle. Permanent ablation of DOT1L results in severe consequences and lethality, but our results revealing sustained proliferation of DOT1L-depleted CMs suggest that temporary inhibition of DOT1L in postnatal hearts might be a strategy to promote re-acquisition of mitotic potential in CMs.

### Experiment No. 2

### Dot1L Inhibition Drives De Novo Proliferation of Adult Cardiomyocytes

Experiment No. 1 shows that ablation of Dot1L in cardiomyocytes from early developmental periods resulted in sustained proliferation of neonatal cardiomyocytes (normally murine cardiomyocytes withdraw from the cell cycle in the first week of life, but Dot1L cKO cardiomyocytes were still proliferating at postnatal day 10). However, it also resulted in a lethal phenotype owing to the essential role DOT1L plays in normal mammalian cardiogenesis and because Dot1L cKO cardiomyocytes were less differentiated than those of control littermates.

Based on these results, Applicant reasoned that conditional ablation of Dot1L in adulthood could be a valid strategy to promote dedifferentiation and de novo proliferation of adult cardiomyocytes, paving the way for novel therapies to promote adult cardiac regeneration. Applicant therefore generated a novel transgenic mouse model based on the cardiomyocyte-specific ablation of Dot1L using the tamoxifen-inducible αMHC-MerCreMer (D. S. Sohal et al., Circ Res 89, (2001)). In these animals, Dot1L is unaffected through embryogenesis and neonatal development, allowing normal cardiogenesis. At 8-weeks-old, animals were induced with intraperitoneal injections of tamoxifen (FIG. 16A) that led to highly efficient Dot1L ablation only in cardiomyocytes (FIG. 16B). As expected, conditional ablation of Dot1L led to a very significant reduction of H3K79me2 levels (FIG. 16C). This downregulation was already detectable at 1-month post-tamoxifen, but became even more evident at 2-months post-induction (FIG. 16C). Inducible Dot1L cKO animals (from here on simply designated as iKO) had normal survival until approximately 7 months post-tamoxifen ablation. At this timepoint, iKO animals started exhibiting a lethal phenotype with all iKO animals dying before 11-months post-induction (FIG. 16D). Whole organ imaging of control and iKO hearts at distinct timepoints (FIG. 16E) and quantification of heart weight/body weight ratios (FIG. 16F) revealed iKO hearts were significantly larger than controls. This was already observed at 1-month post-tamoxifen but became more evident as animals aged. Importantly, differences in heart weight/body weight ratios were exclusively due to increased heart weight in iKOs (FIG. 16G), as average body weight was similar in both genotype groups (FIG. 1611).

Non-invasive echocardiographic assessment of cardiac function confirmed an increased left ventricular mass/body weight ratio (LVM/BW) in iKOs versus controls starting from 2-months post-tamoxifen as well as increased LVPW and IVS in diastole (FIG. 17A). At 4- and 8-months post-tamoxifen this was also accompanied by compromised cardiac function as shown by a significantly reduced fractional shortening (FS, FIG. 17A) that was not detectable at 2-months post-tamoxifen. 8-months post-tamoxifen, iKO hearts also exhibited signs of left ventricular dilation, as shown by increased systolic left ventricular inner diameter (LVIDs). A histological time course analysis of control and iKO hearts (FIG. 17B) provided further insight into the dynamics of the iKO phenotype. In early remodeling (up to two-month post tamoxifen), iKO hearts had significantly thicker ventricular walls (arrows in FIG. 17B) without any signs of chamber dilatation or organ disfunction. However, as animals aged, this initial response transitioned to a second phenotypic manifestation in which the ventricles of iKOs started exhibiting significant dilation (coinciding with the loss of cardiac function determined by echocardiography). Altogether these observations suggest that, whereas the first months post-ablation of Dot1L in adult cardiomyocytes are characterized by an increase in ventricular thickness with sustained cardiac function, continued absence of DOT1L and H3K79me lead to excessive dedifferentiation of cardiomyocytes and consequent progressive loss of cardiac contractile capacity.

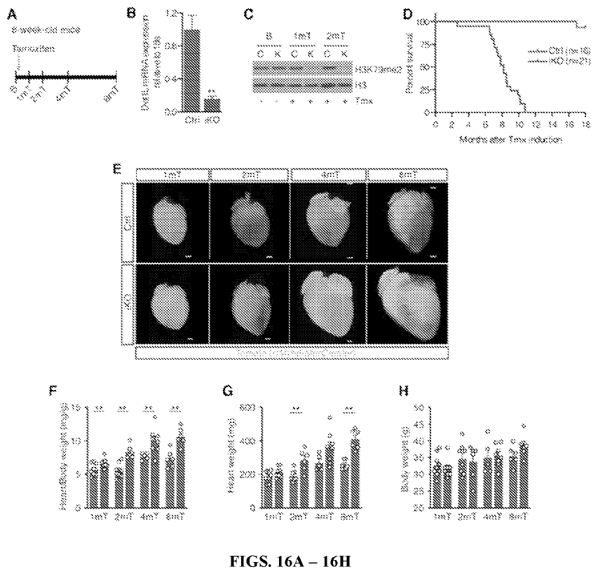

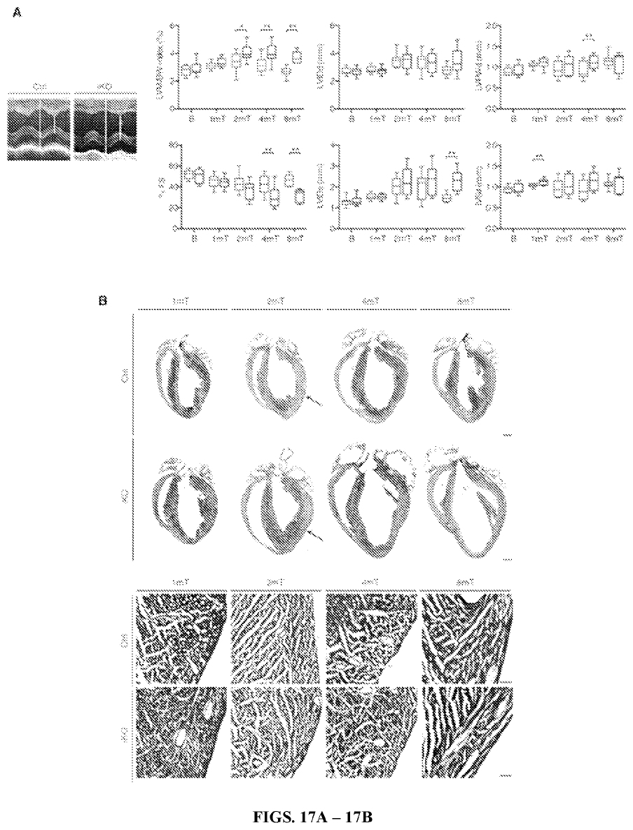

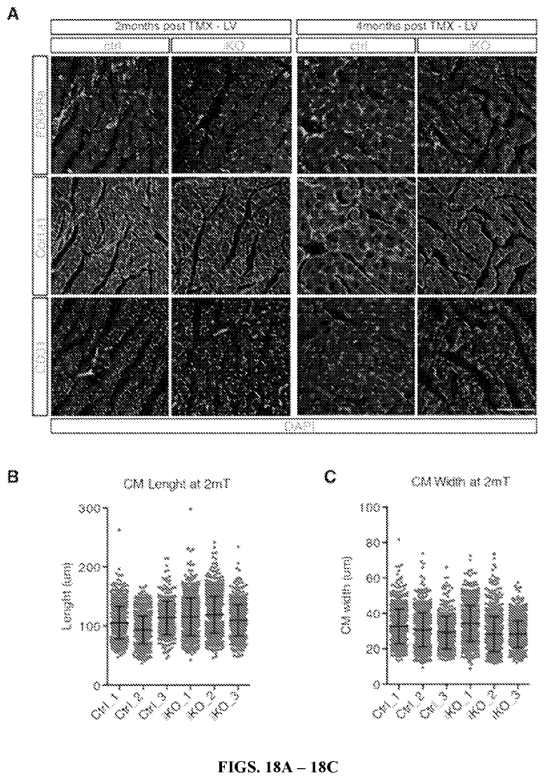

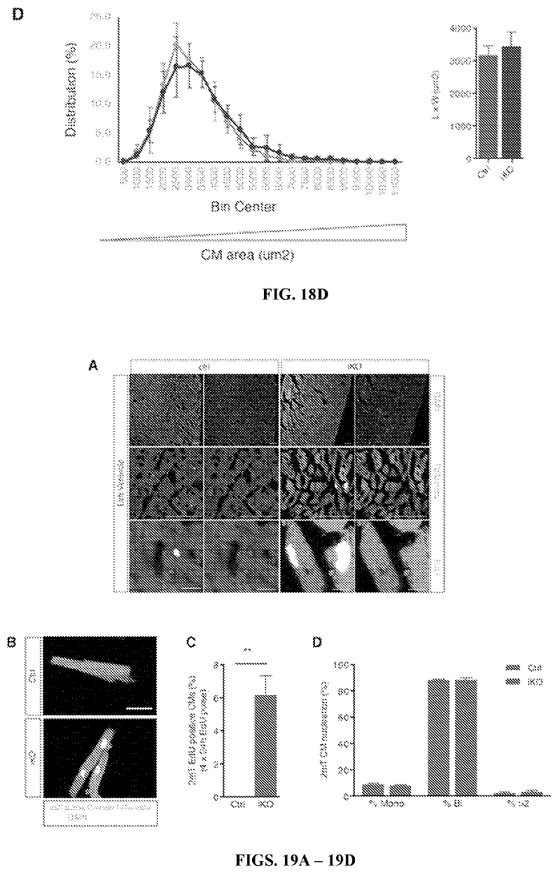

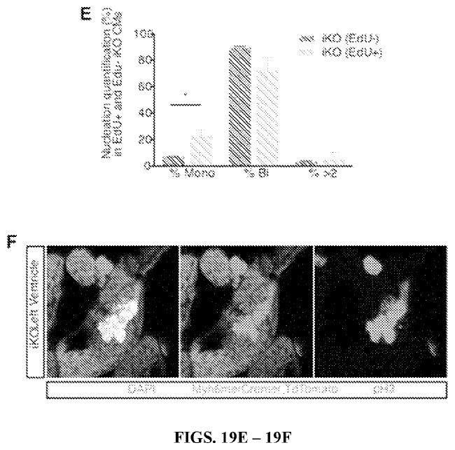

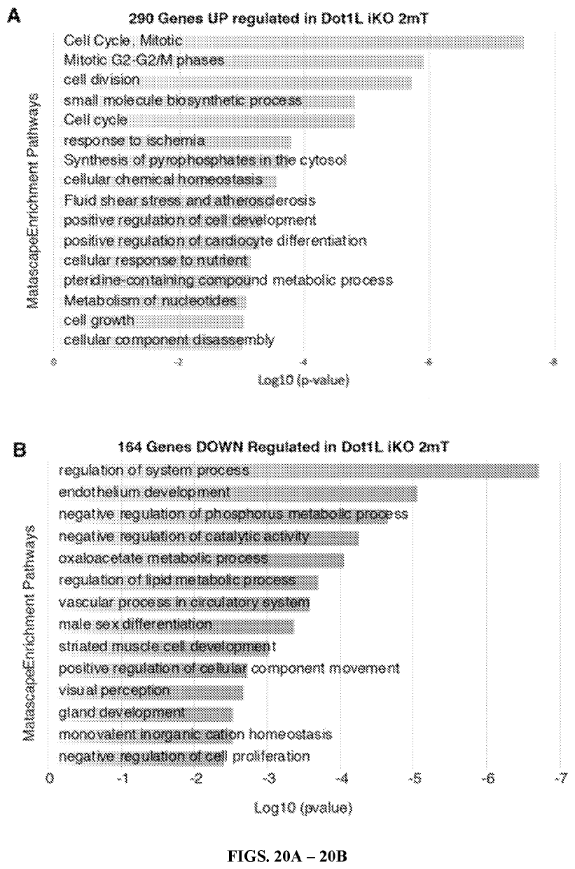

The initial thickening of the myocardial wall at 2-months post-tamoxifen could be due to multiple factors, including: i) activation of fibroblasts and consequent fibrosis, ii) alterations in the vascular network, iii) hypertrophy of cardiomyocytes, or iv) proliferation of cardiomyocytes. Therefore, Applicant tested all these possible contributing factors. Histological analyses (FIG. 18A) clearly showed that, compared with littermate controls, iKOs had a similar abundance of fibroblasts (PDGFRα+) and endothelial cells (CD31+). Consistent with absence of increased fibroblast content, these animals also showed no evidence of increased fibrosis as denoted by Collagen1a1 immunofluorescence (FIG. 18A). To test if cardiomyocyte hypertrophy was contributing to the thickening of the myocardial wall, Applicant quantified the size of ventricular cardiomyocytes isolated from control and iKO hearts at 2-months post-tamoxifen. These analyses revealed that cardiomyocytes of both genotypic groups had similar length and width (FIGS. 18B-18D), therefore excluding cardiomyocyte hypertrophy as the cause for the observed thickening of the myocardial wall at 2-months post-tamoxifen.

Based on the absence of significant contribution from the aforementioned processes, Applicant proceeded with testing the only putative explanation missing: de novo proliferation of cardiomyocytes, with consequent increase in the pool of cardiomyocytes in iKO hearts. To test this, Applicant inferred cardiomyocyte DNA synthesis via assessment of incorporation of the thymidine analog 5-Ethynyl-2′-deoxyuridine (EdU). To facilitate the unequivocal identification of cardiomyocyte nuclei, these analyses were done in animals in which all cardiomyocytes are permanently labeled by the red fluorescent protein tdTomato (permanent genetic labeling resulting from a strategy employing the reporter of Cre activity Rosa26-tdTomato (L. Madisen et al., Nat Neurosci 13, (2010)). Whereas in control hearts the only EdU+ cells detected corresponded to non-myocyte lineages (Edu+, tdTomato-cells), in iKO hearts cardiomyocytes engaging in DNA synthesis (Edu+, tdTomato+ cells) could be easily detected (FIG. 19A). To quantify the extent of this wave of cardiomyocyte-specific DNA synthesis, Applicant analyzed rates of EdU incorporation in cardiomyocytes isolated from the ventricles of iKO and control hearts at 2-months post-tamoxifen (FIG. 19B). Whereas in control hearts there were no EdU+ cardiomyocytes, in iKO hearts about 6% of all cardiomyocytes were EdU positive, potentially indicating a very robust re-entry into cell cycle activity (FIG. 19C). Rather than reflecting mitosis, DNA synthesis in cardiomyocytes can simply be an indication of binucleation. However, quantification of binucleation ratios revealed no differences between iKOs and controls, ruling out this possibility (FIG. 19D). Importantly, quantification of nucleation in EdU+ cells revealed that EdU+ cardiomyocytes were more frequently mononucleated than EdU-cardiomyocytes, further suggesting active mitosis rather than multinucleation (FIG. 19E). Immunostaining of iKO tissue with an antibody recognizing phospho-histone 3, a marker of mitotic cells, clearly showed cardiomyocytes positive for this marker with a DNA (DAPI) pattern consistent with mitotic phases (FIG. 19F).

To further understand molecular mechanisms underlying this phenomenon, Applicant performed RNA-seq analyses assessing the transcriptome of iKO cardiomyocytes. Strikingly, these analyses revealed that, in comparison with controls, iKO cardiomyocytes showed downregulation of relevant metabolic pathways characteristic of mature cardiomyocytes (FIG. 20B), and a highly significant upregulation of genes associated cell cycle (FIG. 20A).

Altogether these data show that conditional ablation of DOT1L is sufficient to promote dedifferentiation and cell cycle re-entry of adult cardiomyocytes, validating Dot1L inhibition as a valid strategy to promote de novo cardiomyocyte proliferation, a desirable feature for cardiac regeneration. In iKO animals, the inhibition of Dot1L takes place in all cardiomyocytes of the heart and in a permanent fashion. This continuous DOT1L ablation contributes to an over-dedifferentiation of cardiomyocytes and consequent ventricular dilation and reduction of cardiac function. Therefore, for therapeutic application, the inhibition of DOT1L can be transient and/or restricted to a subset of cardiomyocytes, rather than taking place throughout the heart. This can be achieved by distinct methods, including but not restricted to: i) the usage of chemical inhibitors of DOT1L activity developed to control this critical epigenetic enzyme, for example EPZ004777, SGC0946, or EPZ-5676. For in vivo use and to the best of Applicant's knowledge, EPZ-5676 is the best rated commercially available inhibitor and can be delivered via daily intraperitoneal injections at a dose of 20 mg/kg/day and optionally monitored ii) use of adeno-associated vectors (AAVs) or any other kind of viral vectors that optionally use cardiomyocyte-specific promoters (for example, the promoter of the gene encoding the contractile protein TroponinT) driving cardiomyocyte-specific expression of a shRNA targeting DOT1L. Examples of target sequences that can be used for shRNA-mediated silencing of human DOT1L include CGCCAACACGAGTGTTATATT (within the coding sequence, clone ID TRCN0000020209) or CACGTTGAACAAGTGCATTTA (within the 3′ untranslated region, clone ID TRCN0000236343). Viral vectors targeting DOT1L can be delivered systemically (cardiomyocyte-specific expression is ensured by the promoter), via intracardiac injection, or delivery during reperfusion of the obstructed coronary artery in a scenario of myocardial infarction. iii) the silencing of DOT1L via siRNAs delivered in lipid nanoparticles, or iv) the application of CRISPR technologies to inhibit transcription or translation of Dot1L or its regulatory elements. Crispr strategies to inhibit transcription (also designated CRISPRi) are based on dCas9 (a mutant version of the Cas9 enzyme that lacks endonuclease activity) fused to a repressor domain (for example the Kruppel Associated Box—KRAB—domain) that is guided to the DOT1L gene by a single guide RNA designed to specifically target the promoter or exons of this gene, thereby repressing its transcription. Crispr regulation of translation, on the other hand, employs a catalytically dead dCas13 that targets RNA molecules (unlike Cas9 that targets DNA). In this case, a guide RNA targeting the translation start site of the DOT1L mRNA is used to ensure that dCas13 selectively represses production of DOT1L protein, without affecting translation of mRNAs encoding other proteins. These strategies can be administered acutely (in a scenario of myocardial infarction) or chronically in any setting of heart failure.

### Equivalents

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

It should be understood that although the present invention has been specifically disclosed by certain aspects, embodiments, and optional features, modification, improvement and variation of such aspects, embodiments, and optional features can be resorted to by those skilled in the art, and that such modifications, improvements and variations are considered to be within the scope of this disclosure.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entireties, to the same extent as if each were incorporated by reference individually. In case of conflict, the present specification, including definitions, will control.

Other aspects are set forth within the following claims.

### REFERENCES

1. Reik, W. Stability and flexibility of epigenetic gene regulation in mammalian development. Nature 447, 425-432 (2007). https://doi.org:10.1038/nature05918
2. Singer, M. S. et al. Identification of high-copy disruptors of telomeric silencing in Saccharomyces cerevisiae. Genetics 150, 613-632 (1998).
3. Feng, Q. et al. Methylation of H3-lysine 79 is mediated by a new family of HMTases without a SET domain. Curr Biol 12, 1052-1058 (2002).
4. McLean, C. M., Karemaker, I. D. & van Leeuwen, F. The emerging roles of DOT1L in leukemia and normal development. Leukemia 28, 2131-2138 (2014). https://doi.org:10.1038/leu.2014.169
5. Steger, D. J. et al. DOT1L/KMT4 recruitment and H3K79 methylation are ubiquitously coupled with gene transcription in mammalian cells. Mol Cell Biol 28, 2825-2839 (2008). https://doi.org:10.1128/MCB.02076-07
6. Godfrey, L. et al. DOT1L inhibition reveals a distinct subset of enhancers dependent on H3K79 methylation. Nat Commun 10, 2803 (2019). https://doi.org:10.1038/s41467-019-10844-3
7. Cattaneo, P. et al. DOT1L-mediated H3K79me2 modification critically regulates gene expression during cardiomyocyte differentiation. Cell Death Differ 23, 555-564 (2016). https://doi.org:10.1038/cdd.2014.199
8. Jones, B. et al. The histone H3K79 methyltransferase Dot1L is essential for mammalian development and heterochromatin structure. PLoS Genet 4, e1000190 (2008). https://doi.org:10.1371/journal.pgen.1000190
9. Nguyen, A. T. et al. DOT1L regulates dystrophin expression and is critical for cardiac function. Genes Dev 25, 263-274 (2011). https://doi.org:10.1101/gad.2018511
10. Guimaraes-Camboa, N. et al. HIFIla Represses Cell Stress Pathways to Allow Proliferation of Hypoxic Fetal Cardiomyocytes. Dev Cell 33, 507-521 (2015). https://doi.org:10.1016/j.devcel.2015.04.021
11. Wu, T. et al. PRDM16 Is a Compact Myocardium-Enriched Transcription Factor Required to Maintain Compact Myocardial Cardiomyocyte Identity in Left Ventricle. Circulation 145, 586-602 (2022). https://doi.org:10.1161/CIRCULATIONAHA.121.056666
12. Evans, S. M., Yelon, D., Conlon, F. L. & Kirby, M. L. Myocardial lineage development. Circ Res 107, 1428-1444 (2010). https://doi.org:10.1161/CIRCRESAHA.110.227405
13. Paige, S. L., Plonowska, K., Xu, A. & Wu, S. M. Molecular regulation of cardiomyocyte differentiation. Circ Res 116, 341-353 (2015). https://doi.org:10.1161/CIRCRESAHA.116.302752
14. Olson, E. N. Gene regulatory networks in the evolution and development of the heart. Science 313, 1922-1927 (2006). https://doi.org:10.1126/science.1132292
15. Harvey, R. P. Patterning the vertebrate heart. Nat Rev Genet 3, 544-556 (2002). https://doi.org:10.1038/nrg843
16. Srivastava, D. Making or breaking the heart: from lineage determination to morphogenesis. Cell 126, 1037-1048 (2006). https://doi.org:10.1016/j.cell.2006.09.003
17. Bao, Z. Z., Bruneau, B. G., Seidman, J. G., Seidman, C. E. & Cepko, C. L. Regulation of chamber-specific gene expression in the developing heart by Irx4. Science 283, 1161-1164 (1999). https://doi.org:10.1126/science.283.5405.1161
18. Vincentz, J. W. et al. Variation in a Left Ventricle-Specific Hand1 Enhancer Impairs GATA Transcription Factor Binding and Disrupts Conduction System Development and Function. Circ Res 125, 575-589 (2019). https://doi.org:10.1161/CIRCRESAHA.119.315313
19. Srivastava, D. et al. Regulation of cardiac mesodermal and neural crest development by the bHLH transcription factor, dHAND. Nat Genet 16, 154-160 (1997). https://doi.org:10.1038/ng0697-154
20. Thomas, T., Yamagishi, H., Overbeek, P. A., Olson, E. N. & Srivastava, D. The bHLH factors, dHAND and eHAND, specify pulmonary and systemic cardiac ventricles independent of left-right sidedness. Dev Biol 196, 228-236 (1998). https://doi.org:10.1006/dbio.1998.8849
21. McFadden, D. G. et al. The Hand1 and Hand2 transcription factors regulate expansion of the embryonic cardiac ventricles in a gene dosage-dependent manner. Development 132, 189-201 (2005). https://doi.org:10.1242/dev.01562
22. Gottlieb, P. D. et al. Bop encodes a muscle-restricted protein containing MYND and SET domains and is essential for cardiac differentiation and morphogenesis. Nat Genet 31, 25-32 (2002). https://doi.org:10.1038/ng866
23. Soonpaa, M. H., Kim, K. K., Pajak, L., Franklin, M. & Field, L. J. Cardiomyocyte DNA synthesis and binucleation during murine development. Am J Physiol 271, H2183-2189 (1996). https://doi.org:10.1152/ajpheart.1996.271.5.H2183
24. Breckenridge, R., Kotecha, S., Towers, N., Bennett, M. & Mohun, T. Pan-myocardial expression of Cre recombinase throughout mouse development. Genesis 45, 135-144 (2007). https://doi.org:10.1002/dvg.20275
25. Madisen, L. et al. A robust and high-throughput Cre reporting and characterization system for the whole mouse brain. Nat Neurosci 13, 133-140 (2010). https://doi.org:10.1038/nn.2467
26. Bruneau, B. G. et al. Chamber-specific cardiac expression of Tbx5 and heart defects in Holt-Oram syndrome. Dev Biol 211, 100-108 (1999). https://doi.org:10.1006/dbio.1999.9298
27. Fulco, C. P. et al. Activity-by-contact model of enhancer-promoter regulation from thousands of CRISPR perturbations. Nat Genet 51, 1664-1669 (2019). https://doi.org:10.1038/s41588-019-0538-0
28. Hecker, D., Ardakani, F. B. & Schulz, M. H. The adapted Activity-By-Contact model for enhancer-gene assignment and its application to single-cell data. bioRxiv (2022). https://doi.org/10.1101/2022.01.28.478202
29. Li, G. et al. Extensive promoter-centered chromatin interactions provide a topological basis for transcription regulation. Cell 148, 84-98 (2012). https://doi.org:10.1016/j.cell.2011.12.014
30. Dao, L. T. M. et al. Genome-wide characterization of mammalian promoters with distal enhancer functions. Nat Genet 49, 1073-1081 (2017). https://doi.org:10.1038/ng.3884
31. Rosa-Garrido, M. et al. High-Resolution Mapping of Chromatin Conformation in Cardiac Myocytes Reveals Structural Remodeling of the Epigenome in Heart Failure. Circulation 136, 1613-1625 (2017). https://doi.org:10.1161/CIRCULATIONAHA.117.029430
32. Lai, A. Y. & Wade, P. A. Cancer biology and NuRD: a multifaceted chromatin remodelling complex. Nat Rev Cancer 11, 588-596 (2011). https://doi.org:10.1038/nrc3091
33. Bruneau, B. G. et al. A murine model of Holt-Oram syndrome defines roles of the T-box transcription factor Tbx5 in cardiogenesis and disease. Cell 106, 709-721 (2001). https://doi.org:10.1016/s0092-8674(01)00493-7
34. Mort, R. L. et al. Fucci2a: a bicistronic cell cycle reporter that allows Cre mediated tissue specific expression in mice. Cell Cycle 13, 2681-2696 (2014). https://doi.org:10.4161/15384101.2015.945381
35. Poolman, R. A., Li, J. M., Durand, B. & Brooks, G. Altered expression of cell cycle proteins and prolonged duration of cardiac myocyte hyperplasia in p27KIP1 knockout mice. Circ Res 85, 117-127 (1999). https://doi.org:10.1161/01.res.85.2.117
36. Mahmoud, A. I. et al. Meis1 regulates postnatal cardiomyocyte cell cycle arrest. Nature 497, 249-253 (2013). https://doi.org:10.1038/naturel2054
37. Alam, P. et al. Inhibition of Senescence-Associated Genes Rb1 and Meis2 in Adult Cardiomyocytes Results in Cell Cycle Reentry and Cardiac Repair Post-Myocardial Infarction. J Am Heart Assoc 8, e012089 (2019). https://doi.org:10.1161/JAHA.119.012089
38. Creyghton, M. P. et al. Histone H3K27ac separates active from poised enhancers and predicts developmental state. Proc Natl Acad Sci USA 107, 21931-21936 (2010). https://doi.org:10.1073/pnas.1016071107
39. Guo, Y. & Pu, W. T. Cardiomyocyte Maturation: New Phase in Development. Circ Res 126, 1086-1106 (2020). https://doi.org:10.1161/CIRCRESAHA.119.315862
40. Eschenhagen, T. et al. Cardiomyocyte Regeneration: A Consensus Statement. Circulation 136, 680-686 (2017). https://doi.org:10.1161/CIRCULATIONAHA.117.029343
41. Louch, W. E., Sheehan, K. A. & Wolska, B. M. Methods in cardiomyocyte isolation, culture, and gene transfer. J Mol Cell Cardiol 51, 288-298 (2011). https://doi.org:10.1016/j.yjmcc.2011.06.012
42. Heinz, S. et al. Transcription Elongation Can Affect Genome 3D Structure. Cell 174, 1522-1536.e1522 (2018). https://doi.org:10.1016/j.cell.2018.07.047
43. Texari, L. et al. An optimized protocol for rapid, sensitive and robust on-bead ChIP-seq from primary cells. STAR Protoc 2, 100358 (2021). https://doi.org:10.1016/j.xpro.2021.100358
44. Dobin, A. et al. STAR: ultrafast universal RNA-seq aligner. Bioinformatics 29, 15-21 (2013). https://doi.org:10.1093/bioinformatics/bts635
45. Robinson, M. D., McCarthy, D. J. & Smyth, G. K. edgeR: a Bioconductor package for differential expression analysis of digital gene expression data. Bioinformatics 26, 139-140 (2010). https://doi.org:10.1093/bioinformatics/btp616
46. Zhou, Y. et al. Metascape provides a biologist-oriented resource for the analysis of systems-level datasets. Nat Commun 10, 1523 (2019). https://doi.org:10.1038/s41467-019-09234-6
47. Langmead, B. & Salzberg, S. L. Fast gapped-read alignment with Bowtie 2. Nat Methods 9, 357-359 (2012). https://doi.org:10.1038/nmeth.1923
48. Li, H. et al. The Sequence Alignment/Map format and SAMtools. Bioinformatics 25, 2078-2079 (2009). https://doi.org:10.1093/bioinformatics/btp352
49. Zhang, Y. et al. Model-based analysis of ChIP-Seq (MACS). Genome Biol 9, R137 (2008). https://doi.org:10.1186/gb-2008-9-9-r137
50. Ross-Innes, C. S. et al. Differential oestrogen receptor binding is associated with clinical outcome in breast cancer. Nature 481, 389-393 (2012). https://doi.org:10.1038/nature10730
51. Quinlan, A. R. & Hall, I. M. BEDTools: a flexible suite of utilities for comparing genomic features. Bioinformatics 26, 841-842 (2010). https://doi.org:10.1093/bioinformatics/btq033
52. Ramirez, F. et al. deepTools2: a next generation web server for deep-sequencing data analysis. Nucleic Acids Res 44, W160-165 (2016). https://doi.org:10.1093/nar/gkw257
53. Durand, N. C. et al. Juicer Provides a One-Click System for Analyzing Loop-Resolution Hi-C Experiments. Cell Syst 3, 95-98 (2016). https://doi.org:10.1016/j.cels.2016.07.002
54. Fornes, O. et al. JASPAR 2020: update of the open-access database of transcription factor binding profiles. Nucleic Acids Res 48, D87-D92 (2020). https://doi.org:10.1093/nar/gkz1001
55. Sobreira, T. J., Durham, A. M. & Gruber, A. TRAP: automated classification, quantification and annotation of tandemly repeated sequences. Bioinformatics 22, 361-362 (2006). https://doi.org:10.1093/bioinformatics/bti809
56. Benjamini, Y. & Hochberg, Y. Controlling the False Discovery Rate: A Practical and Powerful Approach to Multiple Testing. Journal of the Royal Statistical Society: Series B (Methodological) 57, 289-300 (1995). https://doi.org/10.1111/j.2517-6161.1995.tb02031.x

1. A method to modulate cardiogenesis in a mammalian cardiac cell or mammalian cardiac progenitor cell, comprising contacting the cell with a DOT1L gene modulator.

2. The method of claim 1, wherein the DOT1L gene modulator is an agent that upregulates DOT1L gene expression.

3. The method of claim 1, wherein the DOT1L gene modulator is an agent that downregulates or abolishes DOT1L gene expression or function.

4. The method of claim 1, wherein the gene modulator is a polynucleotide encoding a DOT1L protein.

5. The method of claim 1, wherein the gene modulator is a system that reduces or abrogates endogenous DOT1L gene expression or function.

6. The method of claim 5, wherein the gene modulator that reduces or abrogates endogenous DOT1L gene expression or function is selected from a chemical inhibitor of DOT1L activity, shRNA that targets DOT1L under the control of a cardiac-specific promoter, siRNA that targets DOT1L, or CRISPR gene editing that downregulates DOT1L.

7. The method of claim 1, wherein the mammalian cardiac cell is a cell selected from a canine cardiac cell, an equine cardiac cell, a feline cardiac cell, a murine cardiac cell or a human cardiac cell.

8. A method of promoting cardiac regeneration or de novo cell cycle of a post-mitotic mammalian cardiac cell, comprising contacting the cardiac cell with an agent that inhibits expression of an endogenous DOT1L gene or function in the cell, thereby promoting cardiac regeneration in the cell or de novo cell cycle of a post-mitotic cardiac cell.

9. The method of claim 8, wherein the cardiac cell is a cell post-injury.

10. The method of claim 8, wherein the agent reduces or abrogates endogenous DOT1L gene expression in the cell.

11. The method of any of claim 8, wherein the mammalian cardiac cell is a cell selected from a canine cardiac cell, an equine cardiac cell, a feline cardiac cell, a murine cardiac cell or a human cardiac cell.

12. The method of claim 1, wherein the contacting is in vitro or in vivo.

13. The method of claim 8, wherein the gene modulator that reduces or abrogates endogenous DOT1L gene expression or function is selected from a chemical inhibitor of DOT1L activity, shRNA that targets DOT1L under the control of a cardiac-specific promoter, siRNA that targets DOT1L, or CRISPR gene editing that downregulates DOT1L.

14. A method of promoting cardiac regeneration or treating cardiac injury or disease in a subject in need thereof, comprising administering to the subject an agent that inhibits expression of an endogenous DOT1L gene in a mammalian cardiac cell in the subject, thereby promoting cardiac regeneration or treating cardiac injury or disease in the subject.

15. The method of claim 14, wherein the cardiac cell is a cell post-injury.

16. The method of claim 13, wherein the agent reduces or abrogates endogenous DOT1L gene expression in the cell.

17. The method of claim 16, wherein the agent that inhibits expression of the endogenous DOT1L gene expression or function is selected from a chemical inhibitor of DOT1L activity, shRNA that targets DOT1L under the control of a cardiac-specific promoter, siRNA that targets DOT1L, or CRISPR gene editing that downregulates DOT1L.

18. The method of claim 14, wherein the agent is administered locally or systemically.

19. The method of claim 17, wherein the agent is administered locally via intracardiac injection or reperfusion.

20. The method of any of claim 14, wherein the subject is selected from a canine, an equine, a feline, a murine or a human.
